SEQUENCE LISTING (8 sequences)

SEQ ID NO: 1 - DNA, 3055 bp, Artificial Sequence
  synthetic construct; mutant Chase ABC
catggagacagacacactcctgctatgggtactgctgctctgggttccaggttccactggtgacaccagcaatcctgcatttgatcctaaaaatctgatgcagtcagaaatttaccattttgcacaaaataacccattagcagacttctcatcagataaaaactcaatactaacgttatctgataaacgtagcattatgggaaaccaatctcttttatggaaatggaaaggtggtagtagctttactttacataaaaaactgattgtccccaccgataaagaagcatctaaagcatggggacgctcatctacccccgttttctcattttggctttacaatgaaaaaccgattgatggttatcttactatcgatttcggagaaaaactcatttcaaccagtgaggctcaggcaggctttaaagtaaaattagatttcactggctggcgtgctgtgggagtctctttaaataacgatcttgaaaatcgagagatgaccttaaatgcaaccaatacctcctctgatggtactcaagacagcattgggcgttctttaggtgctaaagtcgatagtattcgttttaaagcgccttctaatgtgagtcagggtgaaatctatatcgaccgtattatgttttctgtcgatgatgctcgctaccaatggtctgattatcaagtaaaaactcgcttatcagaacctgaaattcaatttcacaacgtaaagccacaactacctgtaacacctgaaaatttagcggccattgatcttattcgccaacgtctaattaatgaatttgtcggaggtgaaaaagagacaaacctcgcattagaagagcagatcagcaaattaaaaagtgatttcgatgctcttaatattcacactttagcaaatggtggaacgcaaggcagacatctgatcactgataaacaaatcattatttatcaaccagagaatcttaactcccaagataaacaactatttgataattatgttattttaggtcagtacacgacactaatgtttcagattagccgtgcttatgtgctggaaaaagatcccacacaaaaggcgcaactaaagcagatgtacttattaatgacaaagcatttattagatcaaggctttgttaaagggagtgctttagtgacaacccatcactggggatacagttctcgttggtggtatatttccacgttattaatgtctgatgcactaaaagaagcgaacctacaaactcaagtttatgattcattactgtggtattcacgtgagtttaaaagtagttttgatatgaaagtaagtgctgatagctctgatctagattatttcaataccttatctcgccaacatttagccttattattactagagcctgatgatcaaaagcgtatcaacttagttaatactttcagccattatatcactggcgcattaacgcaagtgccaccgggtggtaaagatggtttacgccctgatggtacagcatggcgacatgaaggcaactatccgggctactctttcccagcctttaaaaatgccgctcagctgatttatctgttacgcgatacaccattttcagtgggtgaaagtggttggaataacctgaaaaaagcgatggtttcagcgtggatctacagtaatccagaagttggattaccgcttgcaggaagacacccttttaactcaccttcgttaaaatcagtcgctcaaggctattactggcttgccatgtctgcaaaatcatcgcctgataaaacacttgcatctatttatcttgcgattagtgataaaacacaaaatgaatcaactgctatttttggagaaactattacaccagcgtctttacctcaaggtttctatgcctttaatggcggtgcttttggtattcatcgttggcaagataaaatggtgacactgaaagcttataacaccaatgtttggtcatctgaaatttataacaaagataaccgttatggccgttaccaaagtcatggtgtcgctcaaatagtgagtcagggcgcgcagctgtcacagggctatcagcaagaaggttgggattggaatagaatgcaaggggcaaccactattcaccttcctcttaaagacttagacagtcctaaacctcataccttaatgcaacgtggagagcgtggatttagcggaacatcatcccttgaaggtcaatatggcatgatggcattcgatcttatttatcccgccaatcttgagcgttttgatcctaatttcactgcgaaaaagagtgtattagccgctgataatcacttaatttttattggtagcaatataaatagtagtgataaaaataaaaatgttgaaacgaccttattccaacatgccattactccaacattaaataccctttggattaatggacaaaagatagaaaacatgccttatcaaacaacacttcaacaaggtgattggttaattgatagcaatggcaatggttacttaattactcaagcagaaaaagtaaatgtaagtcgccaacatcaggtttcagcggaaaataaaaatcgccaaccgacagaaggaaactttagctcggcatggatcgatcacagcactcgccccaaagatgccagttatgagtatatggtctttttagatgcgacacctgaaaaaatgggagagatggcacaaaaattccgtgaaaataatgggttatatcaggttcttcgtaaggataaagacgttcatattattctcgataaactcagcaatgtaacgggatatgccttttatcagccagcatcaattgaagacaaatggatcaaaaaggttaataaacctgcaattgtgatgactcatcgacaaaaagacactcttattgtcagtgcagttacacctgatttaaatatgactcgccaaaaagcagcaactcctgtcaccatcaatgtcacgattaatggcaaatggcaatctgctgataaaaatagtgaagtgaaatatcaggtttctggtgataacactgaactgacgtttacgagttactttggtattccacaagaaatcaaactctcgccactcccttga

SEQ ID NO: 2 - DNA, 2991 bp, Artificial Sequence
  synthetic construct; mutant Chase ABC without a signal sequence
accagcaatcctgcatttgatcctaaaaatctgatgcagtcagaaatttaccattttgcacaaaataacccattagcagacttctcatcagataaaaactcaatactaacgttatctgataaacgtagcattatgggaaaccaatctcttttatggaaatggaaaggtggtagtagctttactttacataaaaaactgattgtccccaccgataaagaagcatctaaagcatggggacgctcatctacccccgttttctcattttggctttacaatgaaaaaccgattgatggttatcttactatcgatttcggagaaaaactcatttcaaccagtgaggctcaggcaggctttaaagtaaaattagatttcactggctggcgtgctgtgggagtctctttaaataacgatcttgaaaatcgagagatgaccttaaatgcaaccaatacctcctctgatggtactcaagacagcattgggcgttctttaggtgctaaagtcgatagtattcgttttaaagcgccttctaatgtgagtcagggtgaaatctatatcgaccgtattatgttttctgtcgatgatgctcgctaccaatggtctgattatcaagtaaaaactcgcttatcagaacctgaaattcaatttcacaacgtaaagccacaactacctgtaacacctgaaaatttagcggccattgatcttattcgccaacgtctaattaatgaatttgtcggaggtgaaaaagagacaaacctcgcattagaagagcagatcagcaaattaaaaagtgatttcgatgctcttaatattcacactttagcaaatggtggaacgcaaggcagacatctgatcactgataaacaaatcattatttatcaaccagagaatcttaactcccaagataaacaactatttgataattatgttattttaggtcagtacacgacactaatgtttcagattagccgtgcttatgtgctggaaaaagatcccacacaaaaggcgcaactaaagcagatgtacttattaatgacaaagcatttattagatcaaggctttgttaaagggagtgctttagtgacaacccatcactggggatacagttctcgttggtggtatatttccacgttattaatgtctgatgcactaaaagaagcgaacctacaaactcaagtttatgattcattactgtggtattcacgtgagtttaaaagtagttttgatatgaaagtaagtgctgatagctctgatctagattatttcaataccttatctcgccaacatttagccttattattactagagcctgatgatcaaaagcgtatcaacttagttaatactttcagccattatatcactggcgcattaacgcaagtgccaccgggtggtaaagatggtttacgccctgatggtacagcatggcgacatgaaggcaactatccgggctactctttcccagcctttaaaaatgccgctcagctgatttatctgttacgcgatacaccattttcagtgggtgaaagtggttggaataacctgaaaaaagcgatggtttcagcgtggatctacagtaatccagaagttggattaccgcttgcaggaagacacccttttaactcaccttcgttaaaatcagtcgctcaaggctattactggcttgccatgtctgcaaaatcatcgcctgataaaacacttgcatctatttatcttgcgattagtgataaaacacaaaatgaatcaactgctatttttggagaaactattacaccagcgtctttacctcaaggtttctatgcctttaatggcggtgcttttggtattcatcgttggcaagataaaatggtgacactgaaagcttataacaccaatgtttggtcatctgaaatttataacaaagataaccgttatggccgttaccaaagtcatggtgtcgctcaaatagtgagtcagggcgcgcagctgtcacagggctatcagcaagaaggttgggattggaatagaatgcaaggggcaaccactattcaccttcctcttaaagacttagacagtcctaaacctcataccttaatgcaacgtggagagcgtggatttagcggaacatcatcccttgaaggtcaatatggcatgatggcattcgatcttatttatcccgccaatcttgagcgttttgatcctaatttcactgcgaaaaagagtgtattagccgctgataatcacttaatttttattggtagcaatataaatagtagtgataaaaataaaaatgttgaaacgaccttattccaacatgccattactccaacattaaataccctttggattaatggacaaaagatagaaaacatgccttatcaaacaacacttcaacaaggtgattggttaattgatagcaatggcaatggttacttaattactcaagcagaaaaagtaaatgtaagtcgccaacatcaggtttcagcggaaaataaaaatcgccaaccgacagaaggaaactttagctcggcatggatcgatcacagcactcgccccaaagatgccagttatgagtatatggtctttttagatgcgacacctgaaaaaatgggagagatggcacaaaaattccgtgaaaataatgggttatatcaggttcttcgtaaggataaagacgttcatattattctcgataaactcagcaatgtaacgggatatgccttttatcagccagcatcaattgaagacaaatggatcaaaaaggttaataaacctgcaattgtgatgactcatcgacaaaaagacactcttattgtcagtgcagttacacctgatttaaatatgactcgccaaaaagcagcaactcctgtcaccatcaatgtcacgattaatggcaaatggcaatctgctgataaaaatagtgaagtgaaatatcaggtttctggtgataacactgaactgacgtttacgagttactttggtattccacaagaaatcaaactctcgccactcccttga

SEQ ID NO: 3 - DNA, 61 bp, Artificial Sequence
  synthetic construct; IgKappa sequence of mutant Chase ABC
catggagacagacacactcctgctatgggtactgctgctctgggttccaggttccactggt

SEQ ID NO: 4 - DNA, 2988 bp, Homo sapiens
accagcaacccggcgtttgatccgaaaaacctgatgcagagcgaaatttatcattttgcgcagaacaacccgctggcggattttagcagcgataaaaacagcattctgaccctgagcgataaacgcagcattatgggcaaccagagcctgctgtggaaatggaaaggcggcagcagctttaccctgcataaaaaactgattgtgccgaccgataaagaagcgagcaaagcgtggggccgcagcagcaccccggtgtttagcttttggctgtataacgaaaaaccgattgatggctatctgaccattgattttggcgaaaaactgattagcaccagcgaagcgcaggcgggctttaaagtgaaactggattttaccggctggcgcgcggtgggcgtgagcctgaacaacgatctggaaaaccgcgaaatgaccctgaacgcgaccaacaccagcagcgatggcacccaggatagcattggccgcagcctgggcgcgaaagtggatagcattcgctttaaagcgccgagcaacgtgagccagggcgaaatttatattgatcgcattatgtttagcgtggatgatgcgcgctatcagtggagcgattatcaggtgaaaacccgcctgagcgaaccggaaattcagtttcataacgtgaaaccgcagctgccggtgaccccggaaaacctggcggcgattgatctgattcgccagcgcctgattaacgaatttgtgggcggcgaaaaagaaaccaacctggcgctggaagaaaacattagcaaactgaaaagcgattttgatgcgctgaacattcataccctggcgaacggcggcacccagggccgccatctgattaccgataaacagattattatttatcagccggaaaacctgaacagccaggataaacagctgtttgataactatgtgattctgggcaactataccaccctgatgtttaacattagccgcgcgtatgtgctggaaaaagatccgacccagaaagcgcagctgaaacagatgtatctgctgatgaccaaacatctgctggatcagggctttgtgaaaggcagcgcgctggtgaccacccatcattggggctatagcagccgctggtggtatattagcaccctgctgatgagcgatgcgctgaaagaagcgaacctgcagacccaggtgtatgatagcctgctgtggtatagccgcgaatttaaaagcagctttgatatgaaagtgagcgcggatagcagcgatctggattattttaacaccctgagccgccagcatctggcgctgctgctgctggaaccggatgatcagaaacgcattaacctggtgaacacctttagccattatattaccggcgcgctgacccaggtgccgccgggcggcaaagatggcctgcgcccggatggcaccgcgtggcgccatgaaggcaactatccgggctatagctttccggcgtttaaaaacgcgagccagctgatttatctgctgcgcgataccccgtttagcgtgggcgaaagcggctggaacaacctgaaaaaagcgatggtgagcgcgtggatttatagcaacccggaagtgggcctgccgctggcgggccgccatccgtttaacagcccgagcctgaaaagcgtggcgcagggctattattggctggcgatgagcgcgaaaagcagcccggataaaaccctggcgagcatttatctggcgattagcgataaaacccagaacgaaagcaccgcgatttttggcgaaaccattaccccggcgagcctgccgcagggcttttatgcgtttaacggcggcgcgtttggcattcatcgctggcaggataaaatggtgaccctgaaagcgtataacaccaacgtgtggagcagcgaaatttataacaaagataaccgctatggccgctatcagagccatggcgtggcgcagattgtgagcaacggcagccagctgagccagggctatcagcaggaaggctgggattggaaccgcatgcagggcgcgaccaccattcatctgccgctgaaagatctggatagcccgaaaccgcataccctgatgcagcgcggcgaacgcggctttagcggcaccagcagcctggaaggccagtatggcatgatggcgtttgatctgatttatccggcgaacctggaacgctttgatccgaactttaccgcgaaaaaaagcgtgctggcggcggataaccatctgatttttattggcagcaacattaacagcagcgataaaaacaaaaacgtggaaaccaccctgtttcagcatgcgattaccccgaccctgaacaccctgtggattaacggccagaaaattgaaaacatgccgtatcagaccaccctgcagcagggcgattggctgattgatagcaacggcaacggctatctgattacccaggcggaaaaagtgaacgtgagccgccagcatcaggtgagcgcggaaaacaaaaaccgccagccgaccgaaggcaactttagcagcgcgtggattgatcatagcacccgcccgaaagatgcgagctatgaatatatggtgtttctggatgcgaccccggaaaaaatgggcgaaatggcgcagaaatttcgcgaaaacaacggcctgtatcaggtgctgcgcaaagataaagatgtgcatattattctggataaactgagcaacgtgaccggctatgcgttttatcagccggcgagcattgaagataaatggattaaaaaagtgaacaaaccggcgattgtgatgacccatcgccagaaagataccctgattgtgagcgcggtgaccccggatctgaacatgacccgccagaaagcggcgaccccggtgaccattaacgtgaccattaacggcaaatggcagagcgcggataaaaacagcgaagtgaaatatcaggtgagcggcgataacaccgaactgacctttaccagctattttggcattccgcaggaaattaaactgagcccgctgccg

SEQ ID NO: 5 - protein, 1017 aa, Artificial Sequence
  synthetic construct; mutant Chase ABC plus a signal sequence
METDTLLLWVLLLWVPGSTGDTSNPAFDPKNLMQSEIYHFAQNNPLADFSSDKNSILTLSDKRSIMGNQSLLWKWKGGSSFTLHKKLIVPTDKEASKAWGRSSTPVFSFWLYNEKPIDGYLTIDFGEKLISTSEAQAGFKVKLDFTGWRAVGVSLNNDLENREMTLNATNTSSDGTQDSIGRSLGAKVDSIRFKAPSNVSQGEIYIDRIMFSVDDARYQWSDYQVKTRLSEPEIQFHNVKPQLPVTPENLAAIDLIRQRLINEFVGGEKETNLALEEQISKLKSDFDALNIHTLANGGTQGRHLITDKQIIIYQPENLNSQDKQLFDNYVILGQYTTLMFQISRAYVLEKDPTQKAQLKQMYLLMTKHLLDQGFVKGSALVTTHHWGYSSRWWYISTLLMSDALKEANLQTQVYDSLLWYSREFKSSFDMKVSADSSDLDYFNTLSRQHLALLLLEPDDQKRINLVNTFSHYITGALTQVPPGGKDGLRPDGTAWRHEGNYPGYSFPAFKNAAQLIYLLRDTPFSVGESGWNNLKKAMVSAWIYSNPEVGLPLAGRHPFNSPSLKSVAQGYYWLAMSAKSSPDKTLASIYLAISDKTQNESTAIFGETITPASLPQGFYAFNGGAFGIHRWQDKMVTLKAYNTNVWSSEIYNKDNRYGRYQSHGVAQIVSQGAQLSQGYQQEGWDWNRMQGATTIHLPLKDLDSPKPHTLMQRGERGFSGTSSLEGQYGMMAFDLIYPANLERFDPNFTAKKSVLAADNHLIFIGSNINSSDKNKNVETTLFQHAITPTLNTLWINGQKIENMPYQTTLQQGDWLIDSNGNGYLITQAEKVNVSRQHQVSAENKNRQPTEGNFSSAWIDHSTRPKDASYEYMVFLDATPEKMGEMAQKFRENNGLYQVLRKDKDVHIILDKLSNVTGYAFYQPASIEDKWIKKVNKPAIVMTHRQKDTLIVSAVTPDLNMTRQKAATPVTINVTINGKWQSADKNSEVKYQVSGDNTELTFTSYFGIPQEIKLSPLP

SEQ ID NO: 6 - protein, 996 aa, Artificial Sequence
  synthetic construct; mutant Chase ABC
TSNPAFDPKNLMQSEIYHFAQNNPLADFSSDKNSILTLSDKRSIMGNQSLLWKWKGGSSFTLHKKLIVPTDKEASKAWGRSSTPVFSFWLYNEKPIDGYLTIDFGEKLISTSEAQAGFKVKLDFTGWRAVGVSLNNDLENREMTLNATNTSSDGTQDSIGRSLGAKVDSIRFKAPSNVSQGEIYIDRIMFSVDDARYQWSDYQVKTRLSEPEIQFHNVKPQLPVTPENLAAIDLIRQRLINEFVGGEKETNLALEEQISKLKSDFDALNIHTLANGGTQGRHLITDKQIIIYQPENLNSQDKQLFDNYVILGQYTTLMFQISRAYVLEKDPTQKAQLKQMYLLMTKHLLDQGFVKGSALVTTHHWGYSSRWWYISTLLMSDALKEANLQTQVYDSLLWYSREFKSSFDMKVSADSSDLDYFNTLSRQHLALLLLEPDDQKRINLVNTFSHYITGALTQVPPGGKDGLRPDGTAWRHEGNYPGYSFPAFKNAAQLIYLLRDTPFSVGESGWNNLKKAMVSAWIYSNPEVGLPLAGRHPFNSPSLKSVAQGYYWLAMSAKSSPDKTLASIYLAISDKTQNESTAIFGETITPASLPQGFYAFNGGAFGIHRWQDKMVTLKAYNTNVWSSEIYNKDNRYGRYQSHGVAQIVSQGAQLSQGYQQEGWDWNRMQGATTIHLPLKDLDSPKPHTLMQRGERGFSGTSSLEGQYGMMAFDLIYPANLERFDPNFTAKKSVLAADNHLIFIGSNINSSDKNKNVETTLFQHAITPTLNTLWINGQKIENMPYQTTLQQGDWLIDSNGNGYLITQAEKVNVSRQHQVSAENKNRQPTEGNFSSAWIDHSTRPKDASYEYMVFLDATPEKMGEMAQKFRENNGLYQVLRKDKDVHIILDKLSNVTGYAFYQPASIEDKWIKKVNKPAIVMTHRQKDTLIVSAVTPDLNMTRQKAATPVTINVTINGKWQSADKNSEVKYQVSGDNTELTFTSYFGIPQEIKLSPLP

SEQ ID NO: 7 - protein, 20 aa, Artificial Sequence
  synthetic construct; signal sequence from Igkappa
METDTLLLWVLLLWVPGSTG

SEQ ID NO: 8 - protein, 996 aa, Proteus vulgaris
TSNPAFDPKNLMQSEIYHFAQNNPLADFSSDKNSILTLSDKRSIMGNQSLLWKWKGGSSFTLHKKLIVPTDKEASKAWGRSSTPVFSFWLYNEKPIDGYLTIDFGEKLISTSEAQAGFKVKLDFTGWRAVGVSLNNDLENREMTLNATNTSSDGTQDSIGRSLGAKVDSIRFKAPSNVSQGEIYIDRIMFSVDDARYQWSDYQVKTRLSEPEIQFHNVKPQLPVTPENLAAIDLIRQRLINEFVGGEKETNLALEENISKLKSDFDALNIHTLANGGTQGRHLITDKQIIIYQPENLNSQDKQLFDNYVILGNYTTLMFNISRAYVLEKDPTQKAQLKQMYLLMTKHLLDQGFVKGSALVTTHHWGYSSRWWYISTLLMSDALKEANLQTQVYDSLLWYSREFKSSFDMKVSADSSDLDYFNTLSRQHLALLLLEPDDQKRINLVNTFSHYITGALTQVPPGGKDGLRPDGTAWRHEGNYPGYSFPAFKNASQLIYLLRDTPFSVGESGWNNLKKAMVSAWIYSNPEVGLPLAGRHPFNSPSLKSVAQGYYWLAMSAKSSPDKTLASIYLAISDKTQNESTAIFGETITPASLPQGFYAFNGGAFGIHRWQDKMVTLKAYNTNVWSSEIYNKDNRYGRYQSHGVAQIVSNGSQLSQGYQQEGWDWNRMQGATTIHLPLKDLDSPKPHTLMQRGERGFSGTSSLEGQYGMMAFDLIYPANLERFDPNFTAKKSVLAADNHLIFIGSNINSSDKNKNVETTLFQHAITPTLNTLWINGQKIENMPYQTTLQQGDWLIDSNGNGYLITQAEKVNVSRQHQVSAENKNRQPTEGNFSSAWIDHSTRPKDASYEYMVFLDATPEKMGEMAQKFRENNGLYQVLRKDKDVHIILDKLSNVTGYAFYQPASIEDKWIKKVNKPAIVMTHRQKDTLIVSAVTPDLNMTRQKAATPVTINVTINGKWQSADKNSEVKYQVSGDNTELTFTSYFGIPQEIKLSPLP

(19) United States
(12) Patent Application Publication
(10) Pub. No.: US 2016/0244740 A1
(43) Pub. Date: Aug. 25, 2016

(54) MUTANT CHONDROITINASE ABC I COMPOSITIONS AND METHODS OF THEIR USE

(71) Applicant: OHIO STATE INNOVATION FOUNDATION, Columbus, OH (US)

(72) Inventor: Balveen Kaur, Dublin, OH (US)

(21) Appl. No.: 15/025,036

(22) PCT Filed: Sep. 26, 2014
(86) PCT No.: PCT/US14/57827

§ 371 Date: Mar. 25, 2016

Related U.S. Application Data
(60) Provisional application No. 61/883,436, filed on Sep. 27, 2013.

(51) Int. Cl. C12N 9/88; C12N 7/00; A61K 45/06; A61K 35/763; A61K 48/00

(52) U.S. Cl. CPC C12N 9/88; A61K 35/763; A61K 48/005; A61K 45/06; C12N 7/00; C12Y 402/0202; C12N 2710/16643; A61K 38/00

(57) ABSTRACT

Disclosed are compositions comprising mutant chondroitinase ABC I and methods of their use. Compositions comprising either the nucleic acid or amino acid sequence of mutant chondroitinase ABC I are disclose. Specifically, compositions comprising the nucleic acid sequence of SEQ ID NO:2 or the amino acid sequence of SEQ ID NO:6 are disclosed. The mutant chondroitinase ABC I compositions can be used to treat cancer, increase oncolytic virus spread or treat central nervous system injury.

### CROSS-REFERENCE TO RELATED APPLICATIONS

This application claims benefit of U.S. Provisional Application No. 61/883,436, filed Sep. 27, 2013. Application No. 61/883,436, filed Sep. 27, 2013, is hereby incorporated herein by reference in its entirety.

### STATEMENT REGARDING FEDERALLY SPONSORED RESEARCH

This invention was made with government support under NIH P01 CA163205 awarded by the National Cancer Institute. The government has certain rights in the invention.

### FIELD OF THE INVENTION

The disclosed invention relates generally to mutant Chondroitinase ABC I compositions and methods of using mutant Chondroitinase ABC I for treating cancer and central nervous system injuries.

### BACKGROUND

Oncolytic viruses can be genetically altered or can have a natural propensity to infect or replicate in cancer cells with minimal damage being done to normal cells. Once inside the cancer cells, the oncolytic viruses destroy the cancer cells via the viruses' natural cytolytic property.

Inefficient oncolytic virus dispersal through the tumor extracellular matrix (ECM) can be a significant barrier in its antitumor efficacy (Kaur B, et al. Curr Gene Ther 2009; 9:341-55; Parker et al. Neurotherapeutics 2009; 6:558-69). Structural components of tumor ECM such as collagens and proteoglycans have been shown to hinder distribution of large therapeutic molecules (Netti et al. Cancer Res 2000; 60:2497-503, Zamecnik, J. Acta Neuropathol (Berl) 2005; 110:435-42). There is a need for oncolytic viruses, as well as other cancer therapeutics, that can efficiently spread through the tumor extracellular matrix.

### BRIEF SUMMARY

The present invention provides compositions and methods for providing better infiltration of cancer therapeutics. The compositions include the nucleic acid sequence or the amino acid sequence of a mutant chondroitinase ABC I enzyme.

The present invention also provides methods of treating cancer by administering a compositions that contains the nucleic acid sequence or the amino acid sequence of a mutant chondroitinase ABC I enzyme.

Disclosed are isolated mutant Chondroitinase ABC I proteins. The isolated mutant Chondroitinase ABC I proteins can comprise at least six mutations compared to wild-type Chondroitinase ABC I. The isolated mutant Chondroitinase ABC I protein can comprise the amino acid sequence of SEQ ID NO:6 or SEQ ID NO:5. The isolated mutant Chondroitinase ABC I protein can further comprise at least three codon optimized amino acids. The codon optimized amino acids can be leucine residues. The leucine residues are at least at positions 338, 515 and 518 of SEQ ID NO:5. The isolated mutant Chondroitinase ABC I proteins can further comprise the human Ig κ chain leader sequence. For example, disclosed are isolated mutant Chondroitinase ABC I proteins comprising SEQ ID NO:5.

Disclosed are isolated nucleic acids comprising the sequence of SEQ ID NO:2. Also disclosed are isolated nucleic acids consisting of the sequence of SEQ ID NO:2. The isolated nucleic acids can further comprise the human Ig κ chain leader sequence. Disclosed are isolated nucleic acids comprising the sequence of SEQ ID NO:1.

Disclosed are recombinant proteins comprising the amino acid sequence of SEQ ID NO:6. Also disclosed are recombinant proteins comprising the amino acid sequence of SEQ ID NO:5. Disclosed are recombinant proteins consisting of the amino acid sequence of SEQ ID NO:5.

Disclosed are vectors comprising a nucleic acid comprising the sequence of SEQ ID NO:2. The vector can be a gene expression vector. The vector can be a viral vector. The viral vector can be a herpes simplex virus type-1 (HSV-1).

Disclosed are cells comprising a vector comprising a nucleic acid comprising the sequence of SEQ ID NO:2. The cells can comprise a vector that is a gene expression vector. The vector can be a viral vector. The viral vector can be a herpes simplex virus type-1 (HSV-1). Disclosed are cells comprising a recombinant protein comprising the amino acid sequence of SEQ ID NO:6 or SEQ ID NO:5. Disclosed are cells comprising a recombinant protein consisting of the amino acid sequence of SEQ ID NO:5.

Disclosed are methods of cleaving chondroitin sulfate proteoglycans comprising administering to a subject an effective amount of a composition comprising a nucleic acid sequence, wherein the nucleic acid sequence comprises a mutant Chase ABC nucleic acid sequence that encodes a mutant Chase ABC protein, wherein the Chase ABC protein comprises at least six mutations compared to wild-type Chase ABC.

The methods of cleaving chondroitin sulfate proteoglycans with a mutant Chase ABC can include administering a nucleic acid sequence, wherein the nucleic acid sequence comprises a mutant Chase ABC I protein, wherein the Chase ABC protein comprises the amino acid sequence of SEQ ID NO:6 or SEQ ID NO:5. The mutant Chase ABC I protein can further comprise at least three codon optimized amino acids. The codon optimized amino acids can be leucine residues. The leucine residues can be at least at positions 338, 515 and 518 of SEQ ID NO:5.

The mutant Chase ABC proteins can further comprise a secretion signal, such as the Igκ signal sequence. For example, disclosed are methods of cleaving chondroitin sulfate proteoglycans comprising administering to a subject an effective amount of a composition comprising a nucleic acid sequence, wherein the nucleic acid sequence comprises a mutant Chase ABC nucleic acid sequence that encodes a mutant Chase ABC protein, wherein the Chase ABC protein comprises at least six mutations compared to wild-type Chase ABC, wherein the mutant Chase ABC protein comprises SEQ ID NO:5.

Disclosed are methods of treating cancer comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein.

Also disclosed are methods of treating cancer comprising administering to a subject an effective amount of a composition comprising mutant Chondroitinase ABC I and a cancer therapeutic. The mutant Chondroitinase ABC I can be a nucleic acid sequence, wherein the nucleic acid sequence comprises a mutant Chondroitinase ABC I nucleic acid sequence that encodes a mutant Chondroitinase ABC I protein. In some aspects, the mutant Chondroitinase ABC I is a protein. Compositions used to treat cancer can be formulated to contain both mutant Chondroitinase ABC I, either in DNA or protein form, and a cancer therapeutic.

Disclosed are also compositions comprising mutant Chondroitinase ABC I proteins in a pharmaceutical composition. In some aspects the pharmaceutical composition can further comprises a pharmaceutical carrier, and that the pharmaceutical composition optionally comprises further compounds, such as chemotherapeutic compounds, anti-inflammatory compounds, antiviral compounds and/or immuno-modulating compounds.

In some aspects, the present invention provides pharmaceutical compositions containing (a) one or more compounds of the invention, and (b) one or more chemotherapeutic agents. When used with the compounds of the invention, such chemotherapeutic agents may be used individually, sequentially, or in combination with one or more other such chemotherapeutic agents or in combination with radiotherapy. All chemotherapeutic agents known to a person skilled in the art are here incorporated as combination treatments with compound according to the invention. Other active agents, such as anti-inflammatory drugs, including but not limited to nonsteroidal anti-inflammatory drugs and corticosteroids, antiviral drugs, and immuno-modulating drugs may also be combined in compositions of the invention. Two or more combined compounds may be used together or sequentially.

Also disclosed is a method of using one or more of the compositions disclosed herein for the manufacture of a medicament for the treatment of cancer, wherein said medicament further comprises a chemotherapeutic agent.

Disclosed are methods of treating cancer comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein, wherein the nucleic acid sequence further comprises a secretion signal sequence. The secretion signal sequence can be an Igκ sequence.

Disclosed are methods of treating cancer comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein wherein the nucleic acid can further comprise a secretion signal sequence. The composition can comprise a vector. The vector can comprise the mutant Chondroitinase ABC I nucleic acid sequence. The vector can be a viral vector. The viral vector can be an oncolytic viral vector.

Disclosed are any of the above methods of treating cancer, wherein the mutant Chondroitinase ABC I nucleic acid sequence comprises the nucleic acid sequence of SEQ ID NO:2.

Disclosed are any of the above methods of treating cancer, wherein the mutant Chondroitinase ABC I protein comprises at least six mutations compared to wild-type Chondroitinase ABC I. The mutant Chondroitinase ABC I protein can comprise the amino acid sequence of SEQ ID NO:6. The mutant Chondroitinase ABC I protein can comprise the amino acid sequence of SEQ ID NO:5.

Disclosed are methods of treating cancer comprising administering to a subject an effective amount of a composition comprising a mutant Chondroitinase ABC I protein. The mutant Chondroitinase ABC I protein can comprise at least six mutations compared to wild-type Chondroitinase ABC I. The mutant Chondroitinase ABC I protein can comprise the amino acid sequence of SEQ ID NO:6. The mutant Chondroitinase ABC I protein can comprise the amino acid sequence of SEQ ID NO:5. The mutant Chondroitinase ABC I protein can further comprise at least three codon optimized amino acids. The codon optimized amino acids can be leucine residues. The leucine residues can be at least at positions 338, 515 and 518 of SEQ ID NO:5. For example, disclosed are methods of treating cancer comprising administering to a subject an effective amount of a composition comprising a mutant Chondroitinase ABC I protein, wherein the mutant Chondroitinase ABC I proteins comprises SEQ ID NO:5 or SEQ ID NO:6.

Disclosed are any of the above methods of treating cancer, wherein the mutant Chondroitinase ABC I protein increases the spread of the oncolytic virus.

Disclosed are any of the above methods of treating cancer further comprising administering a cancer therapeutic. The cancer therapeutic can be a chemotherapeutic agent.

Disclosed are methods of increasing the spread of HSV-1 comprising administering to a cell a composition comprising a nucleic acid sequence, wherein the nucleic acid sequence comprises a mutant Chondroitinase ABC I nucleic acid sequence that encodes a mutant Chondroitinase ABC I protein. The composition comprising a nucleic acid sequence can comprise a vector. The vector can comprise the mutant Chondroitinase ABC I nucleic acid sequence. The vector can be a viral vector. The viral vector can be an oncolytic viral vector.

Disclosed are methods of increasing the spread of HSV-1 comprising administering to a cell a composition comprising a nucleic acid sequence, wherein the nucleic acid sequence comprises a mutant Chondroitinase ABC I nucleic acid sequence that encodes a mutant Chondroitinase ABC I protein, wherein the mutant Chondroitinase ABC I nucleic acid sequence comprises the nucleic acid sequence of SEQ ID NO:2. The mutant Chondroitinase ABC I nucleic acid sequence can comprise a sequence capable of encoding a mutant Chondroitinase ABC I protein that comprises at least six mutations compared to wild-type Chondroitinase ABC I. The mutant Chondroitinase ABC I nucleic acid sequence can comprises a sequence capable of encoding a mutant Chondroitinase ABC I protein comprising the amino acid sequence of SEQ ID NO:6 or SEQ ID NO:5.

Disclosed are methods of increasing the spread of HSV-1 comprising administering to a subject an effective amount of a composition comprising a mutant Chondroitinase ABC I protein. The mutant Chondroitinase ABC I protein can comprise at least six mutations compared to wild-type Chondroitinase ABC I. The mutant Chondroitinase ABC I protein can comprise the amino acid sequence of SEQ ID NO:6 or SEQ ID NO:5. The mutant Chondroitinase ABC I protein can further comprise at least three codon optimized amino acids. The codon optimized amino acids can be leucine residues. The leucine residues are at least at positions 338, 515 and 518 of SEQ ID NO:5. For example, disclosed are methods of increasing the spread of HSV-1 comprising administering to a subject an effective amount of a composition comprising a mutant Chondroitinase ABC I protein, wherein the mutant Chondroitinase ABC I proteins comprises SEQ ID NO:5 or SEQ ID NO:6.

Disclosed are methods of increasing the spread of HSV-1 comprising administering to a cell a composition comprising a nucleic acid sequence, wherein the nucleic acid sequence comprises a mutant Chondroitinase ABC I nucleic acid sequence that encodes a mutant Chondroitinase ABC I protein or administering an effective amount of a composition comprising a mutant Chondroitinase ABC I protein. The mutant Chondroitinase ABC I protein can increase the spread of the oncolytic virus.

Disclosed are methods of treating a central nervous system injury comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein. The nucleic acid sequence can further comprises a secretion signal sequence. The secretion signal sequence can be an Igx sequence.

Disclosed are methods of treating a central nervous system injury comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein, wherein the composition comprises a vector. The vector can comprise a mutant Chondroitinase ABC I nucleic acid sequence that encodes a mutant Chondroitinase ABC I protein.

Disclosed are methods of treating a central nervous system injury comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein, wherein the mutant Chondroitinase ABC I nucleic acid sequence comprises the nucleic acid sequence of SEQ ID NO:2.

Disclosed are methods of treating a central nervous system injury comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein, wherein the mutant Chondroitinase ABC I protein comprises at least six mutations compared to wild-type Chondroitinase ABC I.

Disclosed are methods of treating a central nervous system injury comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein, wherein the mutant Chondroitinase ABC I protein comprises the amino acid sequence of SEQ ID NO:6 or SEQ ID NO:5.

Disclosed are methods of treating CNS injury comprising administering to a subject an effective amount of a composition comprising a mutant Chondroitinase ABC I protein. The mutant Chondroitinase ABC I protein can comprise at least six mutations compared to wild-type Chondroitinase ABC I. The mutant Chondroitinase ABC I protein can comprise the amino acid sequence of SEQ ID NO:6 or SEQ ID NO:5.

Disclosed are methods of treating CNS injury comprising administering to a subject an effective amount of a composition comprising a mutant Chondroitinase ABC I protein, wherein the mutant Chondroitinase ABC I protein further comprises at least three codon optimized amino acids. The codon optimized amino acids can be leucine residues. The leucine residues can be at least at positions 338, 515 and 518 of SEQ ID NO:5. For example, disclosed are methods of treating a central nervous system injury comprising administering to a subject an effective amount of a composition comprising a mutant Chondroitinase ABC I protein, wherein the mutant Chondroitinase ABC I proteins comprises SEQ ID NO:5 or SEQ ID NO:6.

Disclosed are methods of treating CNS injury comprising administering to a subject an effective amount of a composition comprising a mutant Chondroitinase ABC I protein, wherein the mutant Chondroitinase ABC I protein increases axon regeneration.

Disclosed are methods of treating CNS injury comprising administering to a subject an effective amount of a composition comprising a mutant Chondroitinase ABC I protein, wherein the central nervous system injury can be a spinal cord injury.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a verification of secretion of active Chondroitinase ABC I. COS-7 cells were transiently transfected with cDNA of either mutated Chase (Chase M) or Chase without mutation (Chase N). Medium from these cells was incubated with CSPG containing medium and functionality of CHASE was tested by western blot for the presence of stubs indicative of Chase function. Western blot analysis (A) showed that only samples transfected with mutated Chase (lane labeled “M”) actively secreted into the medium functional Chase. COS-7 cells were also infected with oncolytic HSV-1 based viruses carrying genes for Chase M or Chase N. Similar analysis as in A showed (B) showed bands only in the samples containing concentrated medium derived from the cells infected with Chase M (lane “M”).

FIG. 2 shows immunoreactivity for stubs left after Chase activity. Athymic mice were injected with GBM 9 cells electroporated with pcDNA3.1D/ChaseM or pcDNA 3.1D/LacZ. After seven days, tumors were removed, fixed and labeled with AbB123-4S antibody. Immunoreactivity was only observed in GBM 9 cells that originated from cells transfected with pcDNA3.1D/ChaseM.

FIG. 3 shows verification of insertion of Chondroitinase ABCI cDNA into the viruses. Verification was performed at the DNA level (A) to visualize restriction patterns of pTransfer shuttle plasmid (pT4/5), BAC plasmid (f), shuttle plasmid with Chase cDNA (pTCh), BAC co-integrant without Chase cDNA (fCon), BAC co-integrant with ChaseN cDNA (fChN), and BAC co-integrant with ChaseM cDNA (fChM). PCR verification of proper Chondroitinase ABC I cDNA insertion into oncolytic HSV vectors was also performed (B).

FIG. 4 shows LN229 spheres treated with different HSVQ viruses and embedded into collagen. LN229 cells were infected with control, Chase N, or Chase M OVs for one hour before being embedded into a collagen solution. Pictures were taken at 24 and 72 hours post infection. OV Chase M showed better spread of OV particles than OV Control and OV Chase N.

FIG. 5 shows increased spread of OV ChaseM compare to OV control after 24 hours in glioma neurosphere models (GBM 2 and GBM 44). Glioma neurospheres were infected with OV Chase M or OV control. GBM 44 (A) and GBM 2 (B) cells were labeled with a red tracker and virus infected cells were labeled green. The overlay of the two signals is shown in yellow. Areas of significant overlay are denoted with arrows.

FIG. 6 shows survival of mice bearing intracranial glioma OGX12 treated with 3×10⁵ pfu of PBS, control virus or OV Chase M 8 days post tumor cell implantation. The figure shows improvement in survival of mice treated with OV Chase M.

FIG. 7 shows reduced aggregation of glioma cells. OG2, OG34, and OG9 cells were transfected with vectors coding for Chase M, Chase N, or control vector. In each cell type, the cells transfected with Chase M showed reduced aggregation compared to Chase N or control.

FIG. 8 shows synergistic effects of inoculating cells with OV Chase M and administering TMZ. OG9 neurospheres were inoculated with OV Control or OV Chase M and dosed with TMZ (temozolamide) four hour later. After five days, MTT assays were performed to determine cell viability and synergy was calculated according to the Chou-Talay method.

FIG. 9 shows caspase 3/7 Activity in human glioma neurospheres after TMZ treatment. OG2, OG34, and OG9 cells were transfected with control vector or Chase M before being treated with TMZ. Transfection with Chase M significantly increased caspase activity in each cell type. *** denotes P<0.0001, * denotes P<0.0013.

FIG. 10 shows sensitivity to TRAIL (TNF-related apoptosis-inducing ligand) in glioma cells after Chase treatment. U87ΔEGFR glioma cells were treated with purified Chondroitinase ABC I, TRAIL, or both. 24 hours later, viable cells were counted using the Trypan exclusion method.

FIG. 11 shows the function of chondroitinase in increasing oncolytic viral spread. (A) Chondroitinases are bacterial enzymes that can cleave a variety of chondroitin sulfate glucosaminoglycans (CS GAGs) covalently attached to the core of CSPGs to tetrasaccharides and dissaccarides without altering the core protein structure. Depicted are some members of the the family of CSPG, and shows the chemical structure of the chondroitin sulfate dimer which constitutes the CSPG chain. (B) Cleavage of CS GAGs allows for effective spread of oncolytic virus between tumor cells.

FIG. 12 shows activity of OV expressing wild type Chase ABC in an organotypic glioma model. U87ΔEGFR human glioma spheroids (made by the hanging drop method), grown on 300-nm organotypic brain slices from 5-7-day-old mice, were infected with 104 pfus of rQNestin34.5, in the presence/absence of Chase ABC. Spread of oHSV in the spheres was visualized by fluorescent imaging of oHSV encoded GFP in infected cells (n=3/group). There was increased oHSV spread within all three glioma spheroids in the presence of purified Chase ABC (bottom panel) as compared to control spheres (top panel). Purified enzyme shows better spread.

FIG. 13 shows the structure of w.t HSV-I (top), 1: rHSVQ1 with gene disrupting insertion of GFP in ICP6 locus and deleted for γ34.5; 2: OV-Chase ABC: encoding for sequence of Chase ABC (Ch) within ICP6 locus of rHSVQ1; 3: rQNestin34.5: virus with nestin driven γ34.5, within the rHSVQ1 backbone and 4: N34.5-Chase ABC with the Chase ABC sequence expressed within the backbone of rQNestin34.5. (diamonds indicate OV chase; Squares indicate OV control; and triangles indicate PBS.

FIG. 14 shows conditioned medium (CM) from U87 and U343 human glioma cells and lysates from human brain and a GBM were collected and concentrated using Centricon filters. This was then treated with Chase ABC or buffer alone. Equal amounts of treated and untreated CM were then analyzed for the presence of “stubs”, generated by Chase ABC digestion of CSPG. Western blot analysis with the BE-123 antibody against the sugar stubs of the peptide moiety of CSPG reveals the presence of high molecular weight bands after Chase ABC treatment of glioma cells (U87 and U343), normal mouse brain and human glioma tissue lysate. This highlights the presence of CSPG in glioma cells.

FIG. 15 shows OV-Chase ABC exhibits improved efficacy against gliomas in vivo: Mice with subcutaneous Gli36ΔEGFR (left panel), and U87EGFR (middle panel) and intracranial U87EGFR (right panel) were injected with PBS (triangles), 10⁶ pfu rHSVQ1 (squares) or 10⁶ pfu OV-Chase ABC (diamonds) as described. Measurement of tumor growth and Kaplan-Meier survival curves, indicate improved anti-tumor efficacy of OV-Chase ABC over control treatments (p<0.05).

FIG. 16 shows A: Fluorescent images of infected U87ΔEGFR spheroids. U87delta EGFR, grown on mouse brain slices infected with 10⁴ pfu of rHSVQ1 or OV-Chase ABC (n=6/group) over a period of time (24 hrs: left, 60 hrs: right) post infection. B: Fluorescent images of G68 (primary neurosphere spheroids infected with 10⁴ pfuof rHSVQ1 or OV-Chase ABC 24 hrs post infection. Note the spread of infectious virus particles into the sphere treated with OVChase ABC compared to control virus.

FIG. 17 shows humanized (mutant) Chase ABC or the w.t Chase ABC were electroporated in Cos 7 cells. The conditioned medium from the transfected cells was incubated with CSPG producing glioma cell CM. Western blot analysis using the Antibody B123 to reveal stubs, created after CSPG digestion by Chase, showed increased stub formation activity in secreted CM derived from cells electroporated with humanized Chase ABC compared to wild type Chase.

FIG. 18 shows an Ex vivo invasion assay: RFP labeled 157GBM tumor neurospheres were treated with PBS (top panel), rHSVQ1 (middle) or OV-Chase (bottom panel) and then implanted in brain slices. RFP labeled cells invading into the brain parenchyma were imaged immediately after OV infection or three days post infection. Note the increased spread of PBS treated RFP labeled cells into the brain slice. Both the OV treated slices showed reduced invasion and more significantly OV-Chase treated spheroids did not show an increase in invasion in this ex vivo model.

FIG. 19 shows OV-Chase ABC did not increase glioma cell dispersal in vivo: Representative photomicrographs of tumor bearing brain sections from nude mice bearing GFP labeled G68 intracranial tumors treated with the indicated virus 10 days post tumor cell implant. 6 days post treatment mice were sacrificed and invasive edges of tumors analyzed for invasion.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a cell” includes a plurality of such cells, reference to “the protein” is a reference to one or more proteins and equivalents thereof known to those skilled in the art, and so forth.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

“Optional” or “optionally” means that the subsequently described event, circumstance, or material may or may not occur or be present, and that the description includes instances where the event, circumstance, or material occurs or is present and instances where it does not occur or is not present.

As used herein, “effective amount” is meant to mean a sufficient amount of the composition to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

As used herein, “treat” is meant to mean administer a composition of the invention to a subject, such as a human or other mammal (for example, an animal model), in order to prevent or delay a worsening of the effects of a disease or condition, or to partially or fully reverse the effects of the disease.

“Variant” sequences have a high degree of sequence similarity. For polynucleotides, conservative variants include those sequences that, because of the degeneracy of the genetic code, encode the amino acid sequence of the mutant chondroitinase ABC I. Variants such as these can be identified with the use of well-known molecular biology techniques, such as, for example, polymerase chain reaction (PCR) and hybridization techniques. Variant polynucleotides also include synthetically derived nucleotide sequences, such as those generated, for example, by using site-directed mutagenesis, but which still encode a functional mutant chondroitinase ABC I enzyme. Generally, variants of a particular polynucleotide will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein. Thus, specifically disclosed are the isolated polynucleotides disclosed herein having 90%, 91%, 92%, 93%, 94%, 95%, 96%, 97%, 98%, 99% or more sequence identity to a polynucleotide encoding SEQ ID NO:2 comprising one or more of the amino acid substitutions of Table 1. In some aspects, the variant sequences would not affect the six amino acids in the mutant chondroitinase ABC I compared to the wild type sequence.

TABLE 1
Amino Acid Substitutions
Original Residue Exemplary
Conservative Substitutions, others are
known in the art.

Ala; Ser
Arg; Lys, Gln
Asn; Gln; His
Asp; Glu
Cys; Ser
Gln; Asn; Lys
Glu; Asp
Gly; Pro
His; Asn; Gln
Ile; Leu; Val
Leu; Ile; Val
Lys; Arg; Gln;
Met; Leu; Ile
Phe; Met; Leu; Tyr
Ser; Thr
Thr; Ser
Trp; Tyr
Tyr; Trp; Phe
Val; Ile; Leu

Variants of a particular polynucleotide can also be evaluated by comparison of the percent sequence identity between the polypeptide encoded by a variant polynucleotide and the polypeptide encoded by the reference polynucleotide. Thus, variants include, for example, isolated polynucleotides that encode a polypeptide with a given percent sequence identity to the mutant chondroitinase ABC I polypeptides set forth herein. Percent sequence identity between any two polypeptides can be calculated using sequence alignment programs and parameters described herein. Where any given pair of polynucleotides is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity. Thus, specifically disclosed are the isolated polypeptides disclosed herein having 90%, 91%, 92%, 93%, 94%, 95%, 96%, 97%, 98%, 99% or more sequence identity to a polypeptide as set forth in SEQ ID NO:2 comprising one or more of the amino acid substitutions of Table 1.

By “variant” polypeptide is intended a polypeptide derived from the mutant chondroitinase ABC I by deletion (so-called truncation) or addition of one or more amino acids to the N-terminal and/or C-terminal end of the polypeptide; deletion or addition of one or more amino acids at one or more sites in the polypeptide; or substitution of one or more amino acids at one or more sites in the polypeptide. Variants of mutant chondroitinase ABC I are biologically active, that is they continue to have the ability to cleave chondroitin sulfate proteoglycans. Such variants can result from, for example, genetic polymorphism or from human manipulation. Biologically active variants of a mutant chondroitinase ABC I will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to the amino acid sequence for the mutant chondroitinase ABC I as determined by sequence alignment programs and parameters described elsewhere herein. A biologically active variant of a polypeptide can differ from that polypeptide by as few as 1-15 amino acid residues, as few as 1-10, such as 6-10, as few as 5, as few as 4, 3, 2, or even 1 amino acid residue.

As used herein, “Inflammatory Disorder” is meant to mean when a subject experiences a cascade of reactions initiated by oxidized lipids in which several cytokine levels go up to alter the normal physiological response. Inflammatory disorders include, but are not limited to Inflammatory Bowel Disease (IBD), systemic lupus erythematosus, Hashimoto's disease, rheumatoid arthritis, graft-versus-host disease, Sjögren's syndrome, pernicious anemia, Addison disease, Alzheimer's disease, scleroderma, Goodpasture's syndrome, ulcerative colitis, Crohn's disease, autoimmune hemolytic anemia, sterility, myasthenia gravis, multiple sclerosis, Basedow's disease, thrombopenia purpura, allergy; asthma, atopic disease, cardiomyopathy, glomerular nephritis, hypoplastic anemia, metabolic syndrome X, peripheral vascular disease, chronic obstructive pulmonary disease (COPD), emphysema, asthma, idiopathic pulmonary fibrosis, pulmonary fibrosis, adult respiratory distress syndrome, osteoporosis, Paget's disease, coronary calcification, polyarteritis nodosa, polymyalgia rheumatica, Wegener's granulomatosis, central nervous system vasculitis (CNSV), Sjogren's syndrome, scleroderma, polymyositis, AIDS inflammatory response, influenza, avian flu, viral pneumonia, endotoxic shock syndrome, sepsis, sepsis syndrome, trauma/wound, corneal ulcer, chronic/non-healing wound, reperfusion injury (prevent and/or treat), ischemic reperfusion injury (prevent and/or treat), spinal cord injuries (mitigating effects), cancers, myeloma/multiple myeloma, ovarian cancer, breast cancer, colon cancer, bone cancer, osteoarthritis, allergic rhinitis, cachexia, Alzheimer's disease, implanted prosthesis, biofilm formation, dermatitis, acute and chronic, eczema, psoriasis, contact dermatitis, erectile dysfunction, macular degeneration, nephropathy, neuropathy, Parkinson's Disease, peripheral vascular disease, and meningitis, cognition and rejection after organ transplantation. Inflammatory diseases can be bacterial, fungal, parasitic and/or viral in nature.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### B. Compositions

1. Chondroitinase ABC I

Chondroitinase ABC I (Chase ABC), or chondroitin-sulfate-ABC endolyase, is a bacterial enzyme responsible for the cleavage of chondroitin sulfate glycosaminoglycans from proteoglycans. Disclosed are nucleic acid and amino acid mutant Chase ABC sequences.

Disclosed are compositions comprising nucleic acid or amino acid mutant Chase ABC sequences. The nucleic acid sequences and amino acid sequences can be isolated, non-naturally occurring, or synthetic. The disclosed nucleic acids that encode mutant Chase ABC proteins and the disclosed mutant Chase ABC proteins retain some level of wild type activity. Regardless of what mutations are present or how the sequence was made, the resulting mutant Chase ABC protein will have the ability to cleave chondroitin sulfate proteoglycans.

Chase ABC, wild type or mutant forms, can be humanized. Humanized Chase ABC can be expressed in mammalian cells such as, but not limited to, primary glioma cells.

i. Nucleic Acid Sequences

Disclosed are isolated nucleic acids comprising the sequence of SEQ ID NO:1. Also disclosed are isolated nucleic acids consisting of the sequence of SEQ ID NO:1. SEQ ID NO:1 includes a mutant Chase ABC nucleic acid sequence.

(SEQ ID NO: 1)
CATGGAGACAGACACACTCCTGCTATGGGTACTGCTGCTCTGGGTTCCAG
GTTCCACTGGTGACACCAGCAATCCTGCATTTGATCCTAAAAATCTGATG
CAGTCAGAAATTTACCATTTTGCACAAAATAACCCATTAGCAGACTTCTC
ATCAGATAAAAACTCAATACTAACGTTATCTGATAAACGTAGCATTATGG
GAAACCAATCTCTTTTATGGAAATGGAAAGGTGGTAGTAGCTTTACTTTA
CATAAAAAACTGATTGTCCCCACCGATAAAGAAGCATCTAAAGCATGGGG
ACGCTCATCTACCCCCGTTTTCTCATTTTGGCTTTACAATGAAAAACCGA
TTGATGGTTATCTTACTATCGATTTCGGAGAAAAACTCATTTCAACCAGT
GAGGCTCAGGCAGGCTTTAAAGTAAAATTAGATTTCACTGGCTGGCGTGC
TGTGGGAGTCTCTTTAAATAACGATCTTGAAAATCGAGAGATGACCTTAA
ATGCAACCAATACCTCCTCTGATGGTACTCAAGACAGCATTGGGCGTTCT
TTAGGTGCTAAAGTCGATAGTATTCGTTTTAAAGCGCCTTCTAATGTGAG
TCAGGGTGAAATCTATATCGACCGTATTATGTTTTCTGTCGATGATGCTC
GCTACCAATGGTCTGATTATCAAGTAAAAACTCGCTTATCAGAACCTGAA
ATTCAATTTCACAACGTAAAGCCACAACTACCTGTAACACCTGAAAATTT
AGCGGCCATTGATCTTATTCGCCAACGTCTAATTAATGAATTTGTCGGAG
GTGAAAAAGAGACAAACCTCGCATTAGAAGAGCAGATCAGCAAATTAAAA
AGTGATTTCGATGCTCTTAATATTCACACTTTAGCAAATGGTGGAACGCA
AGGCAGACATCTGATCACTGATAAACAAATCATTATTTATCAACCAGAGA
ATCTTAACTCCCAAGATAAACAACTATTTGATAATTATGTTATTTTAGGT
CAGTACACGACACTAATGTTTCAGATTAGCCGTGCTTATGTGCTGGAAAA
AGATCCCACACAAAAGGCGCAACTAAAGCAGATGTACTTATTAATGACAA
AGCATTTATTAGATCAAGGCTTTGTTAAAGGGAGTGCTTTAGTGACAACC
CATCACTGGGGATACAGTTCTCGTTGGTGGTATATTTCCACGTTATTAAT
GTCTGATGCACTAAAAGAAGCGAACCTACAAACTCAAGTTTATGATTCAT
TACTGTGGTATTCACGTGAGTTTAAAAGTAGTTTTGATATGAAAGTAAGT
GCTGATAGCTCTGATCTAGATTATTTCAATACCTTATCTCGCCAACATTT
AGCCTTATTATTACTAGAGCCTGATGATCAAAAGCGTATCAACTTAGTTA
ATACTTTCAGCCATTATATCACTGGCGCATTAACGCAAGTGCCACCGGGT
GGTAAAGATGGTTTACGCCCTGATGGTACAGCATGGCGACATGAAGGCAA
CTATCCGGGCTACTCTTTCCCAGCCTTTAAAAATGCCGCTCAGCTGATTT
ATCTGTTACGCGATACACCATTTTCAGTGGGTGAAAGTGGTTGGAATAAC
CTGAAAAAAGCGATGGTTTCAGCGTGGATCTACAGTAATCCAGAAGTTGG
ATTACCGCTTGCAGGAAGACACCCTTTTAACTCACCTTCGTTAAAATCAG
TCGCTCAAGGCTATTACTGGCTTGCCATGTCTGCAAAATCATCGCCTGAT
AAAACACTTGCATCTATTTATCTTGCGATTAGTGATAAAACACAAAATGA
ATCAACTGCTATTTTTGGAGAAACTATTACACCAGCGTCTTTACCTCAAG
GTTTCTATGCCTTTAATGGCGGTGCTTTTGGTATTCATCGTTGGCAAGAT
AAAATGGTGACACTGAAAGCTTATAACACCAATGTTTGGTCATCTGAAAT
TTATAACAAAGATAACCGTTATGGCCGTTACCAAAGTCATGGTGTCGCTC
AAATAGTGAGTCAGGGCGCGCAGCTGTCACAGGGCTATCAGCAAGAAGGT
TGGGATTGGAATAGAATGCAAGGGGCAACCACTATTCACCTTCCTCTTAA
AGACTTAGACAGTCCTAAACCTCATACCTTAATGCAACGTGGAGAGCGTG
GATTTAGCGGAACATCATCCCTTGAAGGTCAATATGGCATGATGGCATTC
GATCTTATTTATCCCGCCAATCTTGAGCGTTTTGATCCTAATTTCACTGC
GAAAAAGAGTGTATTAGCCGCTGATAATCACTTAATTTTTATTGGTAGCA
ATATAAATAGTAGTGATAAAAATAAAAATGTTGAAACGACCTTATTCCAA
CATGCCATTACTCCAACATTAAATACCCTTTGGATTAATGGACAAAAGAT
AGAAAACATGCCTTATCAAACAACACTTCAACAAGGTGATTGGTTAATTG
ATAGCAATGGCAATGGTTACTTAATTACTCAAGCAGAAAAAGTAAATGTA
AGTCGCCAACATCAGGTTTCAGCGGAAAATAAAAATCGCCAACCGACAGA
AGGAAACTTTAGCTCGGCATGGATCGATCACAGCACTCGCCCCAAAGATG
CCAGTTATGAGTATATGGTCTTTTTAGATGCGACACCTGAAAAAATGGGA
GAGATGGCACAAAAATTCCGTGAAAATAATGGGTTATATCAGGTTCTTCG
TAAGGATAAAGACGTTCATATTATTCTCGATAAACTCAGCAATGTAACGG
GATATGCCTTTTATCAGCCAGCATCAATTGAAGACAAATGGATCAAAAAG
GTTAATAAACCTGCAATTGTGATGACTCATCGACAAAAAGACACTCTTAT
TGTCAGTGCAGTTACACCTGATTTAAATATGACTCGCCAAAAAGCAGCAA
CTCCTGTCACCATCAATGTCACGATTAATGGCAAATGGCAATCTGCTGAT
AAAAATAGTGAAGTGAAATATCAGGTTTCTGGTGATAACACTGAACTGAC
GTTTACGAGTTACTTTGGTATTCCACAAGAAATCAAACTCTCGCCACTCC
CTTGA

Nucleotides 1-61 (CATGGAGACAGACACACTCCTGCTATGGGTACTGCT GCTCTGGGTTCCAGGTTCCACTGGT; SEQ ID NO:3) of SEQ ID NO:1 represent an Igκ sequence. Nucleotides 62-64 of SEQ ID NO:1 are left over from the mutation of an EcoRI digestion site. SEQ ID NO:2 is nucleotides 65-3055 of SEQ ID NO:1 and represents the mutant CHASE ABC nucleotide sequence without a signal sequence.

ACCAGCAATCCTGCATTTGATCCTAAAAATCTGATGCAGTCAGAAATTTA
CCATTTTGCACAAAATAACCCATTAGCAGACTTCTCATCAGATAAAAACT
CAATACTAACGTTATCTGATAAACGTAGCATTATGGGAAACCAATCTCTT
TTATGGAAATGGAAAGGTGGTAGTAGCTTTACTTTACATAAAAAACTGAT
TGTCCCCACCGATAAAGAAGCATCTAAAGCATGGGGACGCTCATCTACCC
CCGTTTTCTCATTTTGGCTTTACAATGAAAAACCGATTGATGGTTATCTT
ACTATCGATTTCGGAGAAAAACTCATTTCAACCAGTGAGGCTCAGGCAGG
CTTTAAAGTAAAATTAGATTTCACTGGCTGGCGTGCTGTGGGAGTCTCTT
TAAATAACGATCTTGAAAATCGAGAGATGACCTTAAATGCAACCAATACC
TCCTCTGATGGTACTCAAGACAGCATTGGGCGTTCTTTAGGTGCTAAAGT
CGATAGTATTCGTTTTAAAGCGCCTTCTAATGTGAGTCAGGGTGAAATCT
ATATCGACCGTATTATGTTTTCTGTCGATGATGCTCGCTACCAATGGTCT
GATTATCAAGTAAAAACTCGCTTATCAGAACCTGAAATTCAATTTCACAA
CGTAAAGCCACAACTACCTGTAACACCTGAAAATTTAGCGGCCATTGATC
TTATTCGCCAACGTCTAATTAATGAATTTGTCGGAGGTGAAAAAGAGACA
AACCTCGCATTAGAAGAGCAGATCAGCAAATTAAAAAGTGATTTCGATGC
TCTTAATATTCACACTTTAGCAAATGGTGGAACGCAAGGCAGACATCTGA
TCACTGATAAACAAATCATTATTTATCAACCAGAGAATCTTAACTCCCAA
GATAAACAACTATTTGATAATTATGTTATTTTAGGTCAGTACACGACACT
AATGTTTCAGATTAGCCGTGCTTATGTGCTGGAAAAAGATCCCACACAAA
AGGCGCAACTAAAGCAGATGTACTTATTAATGACAAAGCATTTATTAGAT
CAAGGCTTTGTTAAAGGGAGTGCTTTAGTGACAACCCATCACTGGGGATA
CAGTTCTCGTTGGTGGTATATTTCCACGTTATTAATGTCTGATGCACTAA
AAGAAGCGAACCTACAAACTCAAGTTTATGATTCATTACTGTGGTATTCA
CGTGAGTTTAAAAGTAGTTTTGATATGAAAGTAAGTGCTGATAGCTCTGA
TCTAGATTATTTCAATACCTTATCTCGCCAACATTTAGCCTTATTATTAC
TAGAGCCTGATGATCAAAAGCGTATCAACTTAGTTAATACTTTCAGCCAT
TATATCACTGGCGCATTAACGCAAGTGCCACCGGGTGGTAAAGATGGTTT
ACGCCCTGATGGTACAGCATGGCGACATGAAGGCAACTATCCGGGCTACT
CTTTCCCAGCCTTTAAAAATGCCGCTCAGCTGATTTATCTGTTACGCGAT
ACACCATTTTCAGTGGGTGAAAGTGGTTGGAATAACCTGAAAAAAGCGAT
GGTTTCAGCGTGGATCTACAGTAATCCAGAAGTTGGATTACCGCTTGCAG
GAAGACACCCTTTTAACTCACCTTCGTTAAAATCAGTCGCTCAAGGCTAT
TACTGGCTTGCCATGTCTGCAAAATCATCGCCTGATAAAACACTTGCATC
TATTTATCTTGCGATTAGTGATAAAACACAAAATGAATCAACTGCTATTT
TTGGAGAAACTATTACACCAGCGTCTTTACCTCAAGGTTTCTATGCCTTT
AATGGCGGTGCTTTTGGTATTCATCGTTGGCAAGATAAAATGGTGACACT
GAAAGCTTATAACACCAATGTTTGGTCATCTGAAATTTATAACAAAGATA
ACCGTTATGGCCGTTACCAAAGTCATGGTGTCGCTCAAATAGTGAGTCAG
GGCGCGCAGCTGTCACAGGGCTATCAGCAAGAAGGTTGGGATTGGAATAG
AATGCAAGGGGCAACCACTATTCACCTTCCTCTTAAAGACTTAGACAGTC
CTAAACCTCATACCTTAATGCAACGTGGAGAGCGTGGATTTAGCGGAACA
TCATCCCTTGAAGGTCAATATGGCATGATGGCATTCGATCTTATTTATCC
CGCCAATCTTGAGCGTTTTGATCCTAATTTCACTGCGAAAAAGAGTGTAT
TAGCCGCTGATAATCACTTAATTTTTATTGGTAGCAATATAAATAGTAGT
GATAAAAATAAAAATGTTGAAACGACCTTATTCCAACATGCCATTACTCC
AACATTAAATACCCTTTGGATTAATGGACAAAAGATAGAAAACATGCCTT
ATCAAACAACACTTCAACAAGGTGATTGGTTAATTGATAGCAATGGCAAT
GGTTACTTAATTACTCAAGCAGAAAAAGTAAATGTAAGTCGCCAACATCA
GGTTTCAGCGGAAAATAAAAATCGCCAACCGACAGAAGGAAACTTTAGCT
CGGCATGGATCGATCACAGCACTCGCCCCAAAGATGCCAGTTATGAGTAT
ATGGTCTTTTTAGATGCGACACCTGAAAAAATGGGAGAGATGGCACAAAA
ATTCCGTGAAAATAATGGGTTATATCAGGTTCTTCGTAAGGATAAAGACG
TTCATATTATTCTCGATAAACTCAGCAATGTAACGGGATATGCCTTTTAT
CAGCCAGCATCAATTGAAGACAAATGGATCAAAAAGGTTAATAAACCTGC
AATTGTGATGACTCATCGACAAAAAGACACTCTTATTGTCAGTGCAGTTA
CACCTGATTTAAATATGACTCGCCAAAAAGCAGCAACTCCTGTCACCATC
AATGTCACGATTAATGGCAAATGGCAATCTGCTGATAAAAATAGTGAAGT
GAAATATCAGGTTTCTGGTGATAACACTGAACTGACGTTTACGAGTTACT
TTGGTATTCCACAAGAAATCAAACTCTCGCCACTCCCTTGA

The wild-type sequence for Chase ABC includes the nucleic acid sequence of SEQ ID NO:4.

| (SEQ ID NO: 4) | |
|---|---|
| ACCAGCAACC CGGCGTTTGA TCCGAAAAAC CTGATGCAGA GCGAAATTTA TCATTTTGCG | 60 |
| CAGAACAACC CGCTGGCGGA TTTTAGCAGC GATAAAAACA GCATTCTGAC CCTGAGCGAT | 120 |
| AAACGCAGCA TTATGGGCAA CCAGAGCCTG CTGTGGAAAT GGAAAGGCGG CAGCAGCTTT | 180 |
| ACCCTGCATA AAAAACTGAT TGTGCCGACC GATAAAGAAG CGAGCAAAGC GTGGGGCCGC | 240 |
| AGCAGCACCC CGGTGTTTAG CTTTTGGCTG TATAACGAAA AACCGATTGA TGGCTATCTG | 300 |
| ACCATTGATT TTGGCGAAAA ACTGATTAGC ACCAGCGAAG CGCAGGCGGG CTTTAAAGTG | 360 |
| AAACTGGATT TTACCGGCTG GCGCGCGGTG GGCGTGAGCC TGAACAACGA TCTGGAAAAC | 420 |
| CGCGAAATGA CCCTGAACGC GACCAACACC AGCAGCGATG GCACCCAGGA TAGCATTGGC | 480 |
| CGCAGCCTGG GCGCGAAAGT GGATAGCATT CGCTTTAAAG CGCCGAGCAA CGTGAGCCAG | 540 |
| GGCGAAATTT ATATTGATCG CATTATGTTT AGCGTGGATG ATGCGCGCTA TCAGTGGAGC | 600 |
| GATTATCAGG TGAAAACCCG CCTGAGCGAA CCGGAAATTC AGTTTCATAA CGTGAAACCG | 660 |
| CAGCTGCCGG TGACCCCGGA AAACCTGGCG GCGATTGATC TGATTCGCCA GCGCCTGATT | 720 |
| AACGAATTTG TGGGCGGCGA AAAAGAAACC AACCTGGCGC TGGAAGAAAA CATTAGCAAA | 780 |
| CTGAAAAGCG ATTTTGATGC GCTGAACATT CATACCCTGG CGAACGGCGG CACCCAGGGC | 840 |
| CGCCATCTGA TTACCGATAA ACAGATTATT ATTTATCAGC CGGAAAACCT GAACAGCCAG | 900 |
| GATAAACAGC TGTTTGATAA CTATGTGATT CTGGGCAACT ATACCACCCT GATGTTTAAC | 960 |
| ATTAGCCGCG CGTATGTGCT GGAAAAAGAT CCGACCCAGA AAGCGCAGCT GAAACAGATG | 1020 |
| TATCTGCTGA TGACCAAACA TCTGCTGGAT CAGGGCTTTG TGAAAGGCAG CGCGCTGGTG | 1080 |
| ACCACCCATC ATTGGGGCTA TAGCAGCCGC TGGTGGTATA TTAGCACCCT GCTGATGAGC | 1140 |
| GATGCGCTGA AAGAAGCGAA CCTGCAGACC CAGGTGTATG ATAGCCTGCT GTGGTATAGC | 1200 |
| CGCGAATTTA AAAGCAGCTT TGATATGAAA GTGAGCGCGG ATAGCAGCGA TCTGGATTAT | 1260 |
| TTTAACACCC TGAGCCGCCA GCATCTGGCG CTGCTGCTGC TGGAACCGGA TGATCAGAAA | 1320 |
| CGCATTAACC TGGTGAACAC CTTTAGCCAT TATATTACCG GCGCGCTGAC CCAGGTGCCG | 1380 |
| CCGGGCGGCA AAGATGGCCT GCGCCCGGAT GGCACCGCGT GGCGCCATGA AGGCAACTAT | 1440 |
| CCGGGCTATA GCTTTCCGGC GTTTAAAAAC GCGAGCCAGC TGATTTATCT GCTGCGCGAT | 1500 |
| ACCCCGTTTA GCGTGGGCGA AAGCGGCTGG AACAACCTGA AAAAAGCGAT GGTGAGCGCG | 1560 |
| TGGATTTATA GCAACCCGGA AGTGGGCCTG CCGCTGGCGG GCCGCCATCC GTTTAACAGC | 1620 |
| CCGAGCCTGA AAAGCGTGGC GCAGGGCTAT TATTGGCTGG CGATGAGCGC GAAAAGCAGC | 1680 |
| CCGGATAAAA CCCTGGCGAG CATTTATCTG GCGATTAGCG ATAAAACCCA GAACGAAAGC | 1740 |
| ACCGCGATTT TTGGCGAAAC CATTACCCCG GCGAGCCTGC CGCAGGGCTT TTATGCGTTT | 1800 |
| AACGGCGGCG CGTTTGGCAT TCATCGCTGG CAGGATAAAA TGGTGACCCT GAAAGCGTAT | 1860 |
| AACACCAACG TGTGGAGCAG CGAAATTTAT AACAAAGATA ACCGCTATGG CCGCTATCAG | 1920 |
| AGCCATGGCG TGGCGCAGAT TGTGAGCAAC GGCAGCCAGC TGAGCCAGGG CTATCAGCAG | 1980 |
| GAAGGCTGGG ATTGGAACCG CATGCAGGGC GCGACCACCA TTCATCTGCC GCTGAAAGAT | 2040 |
| CTGGATAGCC CGAAACCGCA TACCCTGATG CAGCGCGGCG AACGCGGCTT TAGCGGCACC | 2100 |
| AGCAGCCTGG AAGGCCAGTA TGGCATGATG GCGTTTGATC TGATTTATCC GGCGAACCTG | 2160 |
| GAACGCTTTG ATCCGAACTT TACCGCGAAA AAAAGCGTGC TGGCGGCGGA TAACCATCTG | 2220 |
| ATTTTTATTG GCAGCAACAT TAACAGCAGC GATAAAAACA AAAACGTGGA AACCACCCTG | 2280 |
| TTTCAGCATG CGATTACCCC GACCCTGAAC ACCCTGTGGA TTAACGGCCA GAAAATTGAA | 2340 |
| AACATGCCGT ATCAGACCAC CCTGCAGCAG GGCGATTGGC TGATTGATAG CAACGGCAAC | 2400 |
| GGCTATCTGA TTACCCAGGC GGAAAAAGTG AACGTGAGCC GCCAGCATCA GGTGAGCGCG | 2460 |
| GAAAACAAAA ACCGCCAGCC GACCGAAGGC AACTTTAGCA GCGCGTGGAT TGATCATAGC | 2520 |
| ACCCGCCCGA AAGATGCGAG CTATGAATAT ATGGTGTTTC TGGATGCGAC CCCGGAAAAA | 2580 |
| ATGGGCGAAA TGGCGCAGAA ATTTCGCGAA AACAACGGCC TGTATCAGGT GCTGCGCAAA | 2640 |
| GATAAAGATG TGCATATTAT TCTGGATAAA CTGAGCAACG TGACCGGCTA TGCGTTTTAT | 2700 |
| CAGCCGGCGA GCATTGAAGA TAAATGGATT AAAAAAGTGA ACAAACCGGC GATTGTGATG | 2760 |
| ACCCATCGCC AGAAAGATAC CCTGATTGTG AGCGCGGTGA CCCCGGATCT GAACATGACC | 2820 |
| CGCCAGAAAG CGGCGACCCC GGTGACCATT AACGTGACCA TTAACGGCAA ATGGCAGAGC | 2880 |
| GCGGATAAAA ACAGCGAAGT GAAATATCAG GTGAGCGGCG ATAACACCGA ACTGACCTTT | 2940 |
| ACCAGCTATT TTGGCATTCC GCAGGAAATT AAACTGAGCC CGCTGCCG | 2988 |

Disclosed are isolated nucleic acid sequences comprising the sequence of SEQ ID NO:2 that are not naturally occurring.

Also disclosed are variants of the mutant Chase ABC nucleotide sequences. Several variants of mutant Chase ABC are disclosed. The disclosed mutant Chase ABC nucleotide sequences encode a functional Chase ABC enzyme. In some aspects, the variations can be mutation. In some aspects, nucleotide mutations other than those that result in the specifically disclosed amino acid sequences can be present.

ii. Amino Acid Sequences

Disclosed are recombinant proteins comprising mutant Chase ABC. Disclosed are recombinant proteins comprising the amino acid sequence of SEQ ID NO:6. SEQ ID NO: 6 is a mutant Chase ABC that corresponds to amino acids 22-1017 of the amino acid sequence of SEQ ID NO:5.

| (SEQ ID NO: 6) | |
|---|---|
| TSNPAFDPKNLMQSEIYHFAQNNPLADFSSD | 31 |
| KNSILTLSDKRSIMGNQSLLWKWKGGSSETLHKKLIVPTDKEASKAWGRSSTPVESEWLY | 91 |
| NEKPIDGYLTIDFGEKLISTSEAQAGFKVKLDFTGWRAVGVSLNNDLENREMTLNATNTS | 151 |
| SDGTQDSIGRSLGAKVDSIRFKAPSNVSQGEIYIDRIMFSVDDARYQWSDYQVKTRLSEP | 211 |
| EIQFHNVKPQLPVTPENLAAIDLIRQRLINEFVGGEKETNLALEEQISKLKSDFDALNIH | 271 |
| TLANGGTQGRHLITDKQIIIYQPENLNSQDKQLFDNYVILGQYTTLMFQISRAYVLEKDP | 331 |
| TQKAQLKQMYLLMTKHLLDQGFVKGSALVTTHHWGYSSRWWYISTLLMSDALKEANLQTQ | 391 |
| VYDSLLWYSREFKSSFDMKVSADSSDLDYFNTLSRQHLALLLLEPDDQKRINLVNTFSHY | 451 |
| ITGALTQVPPGGKDGLRPDGTAWRHEGNYPGYSFPAFKNAAQLIYLLRDTPFSVGESGWN | 511 |
| NLKKAMVSAWIYSNPEVGLPLAGRHPFNSPSLKSVAQGYYWLAMSAKSSPDKTLASIYLA | 571 |
| ISDKTQNESTAIFGETITPASLPQGFYAFNGGAFGIHRWQDKMVTLKAYNTNVWSSEIYN | 631 |
| KDNRYGRYQSHGVAQIVSQGAQLSQGYQQEGWDWNRMQGATTIHLPLKDLDSPKPHTLMQ | 691 |
| RGERGFSGTSSLEGQYGMMAFDLIYPANLERFDPNFTAKKSVLAADNHLIFIGSNINSSD | 751 |
| KNKNVETTLFQHAITPTLNTLWINGQKIENMPYQTTLQQGDWLIDSNGNGYLITQAEKVN | 811 |
| VSRQHQVSAENKNRQPTEGNFSSAWIDHSTRPKDASYEYMVFLDATPEKMGEMAQKFREN | 871 |
| NGLYQVLRKDKDVHIILDKLSNVTGYAFYQPASIEDKWIKKVNKPAIVMTHRQKDTLIVS | 931 |
| AVTPDLNMTRQKAATPVTINVTINGKWQSADKNSEVKYQVSGDNTELTFTSYFGIPQEIK | 991 |
| LSPLP | |

The recombinant proteins that contain the amino acid sequence of SEQ ID NO:6 can be used to cleave chondroitin sulphate proteoglycans. Recombinant proteins comprising the amino acid sequence of SEQ ID NO:5 can cleave chondroitin sulphate proteoglycans.

Disclosed are recombinant proteins comprising the amino acid sequence of SEQ ID NO:5. In some aspects, the recombinant proteins can consist of the amino acid sequence of SEQ ID NO:5. SEQ ID NO:5 comprises SEQ ID NO:6 (mutant CHASE ABC) and a signal sequence to aid in secretion.

| (SEQ ID NO: 5) | |
|---|---|
| METDTLLLWVLLLWVPGSTGDTSNPAFDPKNLMQSEIYHFAQNNPLADFSSD | 52 |
| KNSILTLSDKRSIMGNQSLLWKWKGGSSFTLHKKLIVPTDKEASKAWGRSSTPVFSFWLY | 112 |
| NEKPIDGYLTIDFGEKLISTSEAQAGFKVKLDFTGWRAVGVSLNNDLENREMTLNATNTS | 172 |
| SDGTQDSIGRSLGAKVDSIRFKAPSNVSQGEIYIDRIMFSVDDARYQWSDYQVKTRLSEP | 232 |
| EIQFHNVKPQLPVTPENLAAIDLIRQRLINEFVGGEKETNLALEEQISKLKSDFDALNIH | 292 |
| TLANGGTQGRHLITDKQIIIYQPENLNSQDKQLFDNYVILGQYTTLMFQISRAYVLEKDP | 352 |
| TQKAQLKQMYLLMTKHLLDQGFVKGSALVTTHHWGYSSRWWYISTLLMSDALKEANLQTQ | 412 |
| VYDSLLWYSREFKSSFDMKVSADSSDLDYFNTLSRQHLALLLLEPDDQKRINLVNTFSHY | 472 |
| ITGALTQVPPGGKDGLRPDGTAWRHEGNYPGYSFPAFKNAAQLIYLLRDTPFSVGESGWN | 532 |
| NLKKAMVSAWIYSNPEVGLPLAGRHPFNSPSLKSVAQGYYWLAMSAKSSPDKTLASIYLA | 592 |
| ISDKTQNESTAIFGETITPASLPQGFYAFNGGAFGIHRWQDKMVTLKAYNTNVWSSEIYN | 652 |
| KDNRYGRYQSHGVAQIVSQGAQLSQGYQQEGWDWNRMQGATTIHLPLKDLDSPKPHTLMQ | 712 |
| RGERGFSGTSSLEGQYGMMAFDLIYPANLERFDPNFTAKKSVLAADNHLIFIGSNINSSD | 772 |
| KNKNVETTLFQHAITPTLNTLWINGQKIENMPYQTTLQQGDWLIDSNGNGYLITQAEKVN | 832 |
| VSRQHQVSAENKNRQPTEGNFSSAWIDHSTRPKDASYEYMVFLDATPEKMGEMAQKFREN | 892 |
| NGLYQVLRKDKDVHIILDKLSNVTGYAFYQPASIEDKWIKKVNKPAIVMTHRQKDTLIVS | 952 |
| AVTPDLNMTRQKAATPVTINVTINGKWQSADKNSEVKYQVSGDNTELTFTSYFGIPQEIK | 1012 |
| LSPLP | |

Amino acids 1-20 (METDTLLLWVLLLWVPGSTG; SEQ ID NO:7) of SEQ ID NO:5 are the signal sequence from human Ig κ. The aspartic acid at amino acid 21 is produced from the nucleic acids left over from the EcoRI cleavage during cloning Amino acids 22-1017 of the amino acid sequence of SEQ ID NO:5 correspond to SEQ ID NO:6 which is the mutant chondroitinase sequence. Amino acids Q257, Q313, Q320, A492, Q650, and A652 in SEQ ID NO:6 correspond to amino acids Q278, Q334, Q345, A513, Q671 and A673 of SEQ ID NO:5. The amino acid sequences are twenty-one amino acids off between SEQ ID NO:6 and SEQ ID NO:5 because SEQ ID NO:6 is only the mutant Chase ABC sequence and SEQ ID NO:5 contains an Igκ sequence and a cleavage site 3′ to the mutant Chase ABC sequence.

The presence or absence of amino acid 21 of SEQ ID NO:5 is not necessary for the function of the enzyme. In some instances, the aspartic acid at amino acid 21 is absent. In some instances, there is a longer linker between the signal sequence from human Ig κ and the mutant chondroitinase sequence.

Disclosed are recombinant proteins comprising the amino acid sequence of SEQ ID NO:6 and further comprising a secretion signal. The secretion signal can be the human Igκ signal. Other secretion signals can be a secretion signal that appears before any cellular protein that is secreted by the classical secretion pathway such as but not limited to BAI1 MMP, and IgG.

SEQ ID NO:8 is the wild type Chase ABC amino acid sequence from Proteus vulgaris. The wild type Chase ABC (SEQ ID NO:8) is identical to the mutant Chase ABC (SEQ ID NO:6) with the exception of the following mutations: position 257 of SEQ ID NO:6 is Q instead of N; position 313 of SEQ ID NO:6 is Q instead of N; position 320 of SEQ ID NO:6 is Q instead of N, position 492 of SEQ ID NO:6 is A instead of S, position 650 of SEQ ID NO:6 is Q instead of N, and position 652 of SEQ ID NO:6 is A instead of S. SEQ ID NO:8 can have any amino acid substitution that retains enzyme function. In some instances a conserved amino acid substitution can be used. In some instances a non-conserved amino acid substitution can be used as long as the substitution results in a functional enzyme.

Amino acids 26-1021 of the chondroitinase ABC I sequence from Proteus vulgaris are shown as SEQ ID NO:8.

(SE0 ID NO: 8)
TSNPA FDPKNLMQSE IYHFAQNNPL ADFSSDKNSI LTLSDKRSIM
GNQSLLWKWK GGSSFTLHKK LIVPTDKEAS KAWGRSSTPV
FSFWLYNEKP IDGYLTIDFG EKLISTSEAQ AGFKVKLDFT
GWRAVGVSLN NDLENREMTL NATNTSSDGT QDSIGRSLGA
KVDSIRFKAP SNVSQGEIYI DRIMFSVDDA RYQWSDYQVK
TRLSEPEIQF HNVKPQLPVT PENLAAIDLI RQRLINEFVG
GEKETNLALE ENISKLKSDF DALNIHTLAN GGTQGRHLIT
DKQIIIYQPE NLNSQDKQLF DNYVILGNYT TLMFNISRAY
VLEKDPTQKA QLKQMYLLMT KHLLDQGFVK GSALVTTHHW
GYSSRWWYIS TLLMSDALKE ANLQTQVYDS LLWYSREFKS
SFDMKVSADS SDLDYFNTLS RQHLALLLLE PDDQKRINLV
NTFSHYITGA LTQVPPGGKD GLRPDGTAWR HEGNYPGYSF
PAFKNASQLI YLLRDTPFSV GESGWNNLKK AMVSAWIYSN
PEVGLPLAGR HPFNSPSLKS VAQGYYWLAM SAKSSPDKTL
ASIYLAISDK TQNESTAIFG ETITPASLPQ GFYAFNGGAF
GIHRWQDKMV TLKAYNTNVW SSEIYNKDNR YGRYQSHGVA
QIVSNGSQLS QGYQQEGWDW NRMQGATTIH LPLKDLDSPK
PHTLMQRGER GFSGTSSLEG QYGMMAFDLI YPANLERFDP
NFTAKKSVLA ADNHLIFIGS NINSSDKNKN VETTLFQHAI
TPTLNTLWIN GQKIENMPYQ TTLQQGDWLI DSNGNGYLIT
QAEKVNVSRQ HQVSAENKNR QPTEGNFSSA WIDHSTRPKD
ASYEYMVFLD ATPEKMGEMA QKFRENNGLY QVLRKDKDVH
IILDKLSNVT GYAFYQPASI EDKWIKKVNK PAIVMTHRQK
DTLIVSAVTP DLNMTRQKAA TPVTINVTIN GKWQSADKNS
EVKYQVSGDN TELTFTSYFG IPQEIKLSPL P

The first 25 amino acids of the chondroitinase ABC I sequence from Proteus vulgaris correspond to the bacterial signal sequence. Because the bacterial signal sequence is not required for mammalian cells, this sequence has been replaced with a mammalian signal sequence in the present studies. For example, SEQ ID NO:5 contains the Igκ signal sequence and the mutant chondroitinase sequence.

Besides the amino acid substitutions, L338, L515 and L518 of SEQ ID NO:5 can be codon improved. The codon improvement can be for mammalian expression. The codon improvements can be changes in the nucleic acid sequence to improve translation without causing an amino acid change.

Disclosed are variants of the mutant chondroitinase ABC I sequence. For example, amino acids besides those at positions 257, 313, 320, 492, 650, and 652 of SEQ ID NO:6 can be mutated as long as the resulting enzyme is functional. The replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

a. Peptide Synthesis

One method of producing proteins is to link two or more peptides or polypeptides together by protein chemistry techniques. For example, peptides or polypeptides can be chemically synthesized using currently available laboratory equipment using either Fmoc (9 fluorenylmethyloxycarbonyl) or Boc (tert butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof (Grant GA (1992) Synthetic Peptides: A User Guide. W.H. Freeman and Co., N.Y. (1992); Bodansky M and Trost B., Ed. (1993) Principles of Peptide Synthesis. Springer Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two-step chemical reaction (Dawson et al. Synthesis of Proteins by Native Chemical Ligation. Science, 266:776 779 (1994)). The first step is the chemoselective reaction of an unprotected synthetic peptide thioester with another unprotected peptide segment containing an amino terminal Cys residue to give a thioester linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark Lewis I et al., J. Biol. Chem., 269:16075 (1994); Clark Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257 267 (1992)).

2. Vectors

Disclosed are vectors comprising a nucleic acid sequence capable of encoding the mutant Chondroitinase ABC I proteins disclosed herein.

Disclosed are vectors comprising a nucleic acid sequence comprising the sequence of SEQ ID NO:2. Also disclosed are vectors comprising a nucleic acid sequence comprising the sequence of SEQ ID NO:1.

The vectors can be a gene expression vector. Gene expression vectors allow for the nucleic acid sequence being delivered to be expressed. Thus, a mutant chondroitinase ABC I would be produced in cells in which these vectors have been delivered.

The vectors can be a viral or non-viral vector. Viral vectors are, for example, herpes simplex virus type-1 (HSV-1), Adenovirus, Adeno-associated virus, Herpes virus, Vaccinia virus, Polio virus, AIDS virus, neuronal trophic virus, Sindbis and other RNA viruses, including viruses with the HIV backbone, as well as lentiviruses. Also preferred are any viral families which share the properties of these viruses which make them suitable for use as vectors. Retroviruses include Murine Maloney Leukemia virus, MMLV, and retroviruses that express the desirable properties of MMLV as a vector.

In some aspects, the viral vector can be an oncolytic viral vector. Disclosed are oncolytic viral vectors that can be used to treat cancer due to their ability to selectively infect or replicate in cancer cells and not normal cells. There are several oncolytic viruses that can be used as viral vectors including but not limited to herpes simplex virus type-1 (HSV-1), adenovirus, poxvirus, measles virus, and vesicular stomatitis virus. HSV-1 and HSV-1 mutants can be used to treat cancer based on the following characteristics: (1) it can infect and replicate in a variety of cell types (including tumor cells of human and rodent origins); (2) it is cytolytic by nature (i.e., during its productive infection cycle it can destroy and lyse the infected cells); (3) the well characterized 152-kb genome contains many non-essential genes that, in theory, can be replaced with multiple therapeutic genes (up to 30 kb in size); (4) antiherpetic drugs (e.g., acyclovir) are available to abort unfavorable viral replication and (5) the virus remains as an episome within the infected cell which precludes insertional mutagenesis (Phuangsab et al. Cancer Lett 172:27-36, 2001).

3. Recombinant Oncolytic Viruses

Disclosed are recombinant oncolytic viruses comprising a nucleic acid sequence, wherein the nucleic acid sequence comprises a mutant Chase ABC sequence.

Disclosed are recombinant oncolytic viruses comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises a sequence capable of encoding a mutant Chase ABC protein that comprises at least six mutations compared to wild-type Chase ABC. For example, the mutant Chase ABC protein can have at least six mutations compared to the amino acid sequence of SEQ ID NO:8.

Disclosed are recombinant oncolytic viruses comprising a nucleic acid sequence, wherein the nucleic acid sequence comprises a mutant Chase ABC sequence further comprising at least three codon optimized amino acids. The codon optimized amino acids can be leucine residues. The leucine residues are at least at positions 338, 515 and 518 of SEQ ID NO:5.

The recombinant oncolytic viruses can further comprise a nucleic acid sequence comprising a secretion signal. The secretion signal can be but is not limited to human Ig κ chain leader sequence. Other secretion signals can be a secretion signal that appears before any cellular protein that is secreted by the classical secretion pathway including, but not limited to, BAI1, MMP, and IgG. For example, disclosed are isolated mutant Chondroitinase ABC I proteins comprising SEQ ID NO:5.

The oncolytic virus can be HSV-1.

The oncolytic viruses can not only target the mutant Chase ABC to cancer cells, but it can also provide therapeutic effects by killing the cancer cells. The presence of the mutant Chase ABC allows for the oncolytic virus to spread more efficiently throughout the tumore extracellular matrix.

4. Cells

Disclosed are cells containing the nucleic acids, proteins and vectors described herein. For example, disclosed are cells containing the nucleic acid of SEQ ID NO:2 or SEQ ID NO:1. Cells containing the nucleic acid of SEQ ID NO:2 can express a protein comprising the amino acid sequence of SEQ ID NO:6. Cells containing the nucleic acid of SEQ ID NO:1 can express a protein comprising the amino acid sequence of SEQ ID NO:5.

Disclosed are cells comprising a vector comprising a nucleic acid comprising the sequence of SEQ ID NO:2. Disclosed are cells comprising a vector comprising a nucleic acid comprising the sequence of SEQ ID NO:1. The vectors present in the cells can be gene expression vectors. In some aspects, the vector can be a viral vector. Examples of viral vectors, include but are not limited to, HSV-1, adenovirus, adeno-associated virus, and lentiviruses.

Disclosed are cells comprising a recombinant protein comprising the amino acid sequence of SEQ ID NO:6. Disclosed are cells comprising a recombinant protein comprising the amino acid sequence of SEQ ID NO:5. Disclosed are cells comprising a recombinant protein consisting of the amino acid sequence of SEQ ID NO:5. In some aspects, the cells secrete a protein having the sequence of SEQ ID NO:6 or SEQ ID NO:5.

### C. Methods

1. Methods of Cleaving CSPGs

Disclosed are methods of cleaving chondroitin sulfate proteoglycans with a mutant Chase ABC. Disclosed are methods of cleaving chondroitin sulfate proteoglycans comprising administering to a subject an effective amount of a composition comprising a nucleic acid sequence, wherein the nucleic acid sequence comprises a mutant Chase ABC nucleic acid sequence that encodes a mutant Chase ABC protein, wherein the Chase ABC protein comprises at least six mutations compared to wild-type Chase ABC.

The methods of cleaving chondroitin sulfate proteoglycans with a mutant Chase ABC can include administering a nucleic acid sequence, wherein the nucleic acid sequence comprises a mutant Chase ABC I protein, wherein the Chase ABC protein comprises the amino acid sequence of SEQ ID NO:6 or SEQ ID NO:5. The mutant Chase ABC I protein can further comprise at least three codon optimized amino acids. The codon optimized amino acids can be leucine residues. The leucine residues can be at least at positions 338, 515 and 518 of SEQ ID NO:5.

The mutant Chase ABC proteins can further comprise a secretion signal. For example, the secretion signal sequence can be an Igκ sequence, BAI1 sequence, MMP sequence or IgG sequence. The secretion signals can allow for effective secretion of the mutant Chondroitinase ABC I protein that is encoded by the nucleic acid sequence. For example, disclosed are isolated mutant Chondroitinase ABC I proteins comprising SEQ ID NO:5.

The compositions used in the methods of cleaving chondroitin sulfate proteoglycans can comprise a vector. In some aspects, the composition can comprise a vector wherein the vector comprises a mutant Chase ABC nucleic acid sequence that encodes a mutant Chase ABC protein. The vector can be a viral or nonviral vector. In some aspects, the vector can be a viral vector. For example, the viral vector can be an oncolytic viral vector. Administering an oncolytic virus can be beneficial because oncolytic viruses target cancer cells. Thus, an oncolytic virus that comprises a nucleic acid sequence wherein the nucleic acid sequence comprises a mutant Chase ABC nucleic acid sequence that encodes a mutant Chase ABC protein can infect cancer cells and the mutant Chase ABC protein can be secreted from the cancer cells and cleave chondroitin sulphate proteoglycans. The cleavage of chondroitin sulphate proteoglycans allows for better infiltration of the oncolytic virus which can ultimately lead to the killing of more cancer cells. The cleavage also allows for better infiltration of cancer therapeutics. Therefore, administering Chase ABC, either as a nucleic acid sequence or protein, can provide better infiltration of cancer therapeutics.

Disclosed are methods of cleaving chondroitin sulfate proteoglycans comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises the nucleic acid sequence of SEQ ID NO:2.

Disclosed are methods of cleaving chondroitin sulfate proteoglycans comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises a sequence capable of encoding a mutant Chase ABC protein comprising the amino acid sequence of SEQ ID NO:6 or SEQ ID NO:5.

In some aspects, the methods of cleaving chondroitin sulfate proteoglycans comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein, wherein the mutant Chondroitinase ABC I nucleic acid sequence comprises a sequence capable of encoding a mutant Chondroitinase ABC I protein that comprises at least six mutations compared to wild-type Chondroitinase ABC I. For example, the mutant Chondroitinase ABC I protein can have at least six mutations compared to the amino acid sequence of SEQ ID NO:8.

Disclosed are methods of cleaving chondroitin sulfate proteoglycans comprising administering to a subject an effective amount of a composition comprising a mutant Chase ABC protein.

The mutant Chase ABC protein present in the compositions used in these methods can comprise at least six mutations compared to wild-type Chase ABC. In some aspects, the mutant Chase ABC protein can comprise the amino acid sequence of SEQ ID NO:6. In some aspects, the mutant Chase ABC protein can comprise the amino acid sequence of SEQ ID NO:5. In some aspects, the mutant Chase ABC protein can further comprise at least three codon optimized amino acids. The codon optimized amino acids can be leucine residues. In some aspects, the leucine residues can be at least at positions 338, 515 and 518 of SEQ ID NO:5, which correspond to positions 342, 519 and 522 of SEQ ID NO:8.

In some aspects, the disclosed methods of cleaving chondroitin sulfate proteoglycans can be used in combination with anti-cancer therapeutics.

The mutant Chase ABC protein of the disclosed methods can increase the spread of an oncolytic virus or allow for better infiltration of cancer therapeutics.

The methods of cleaving chondroitin sulfate proteoglycans can be used as a method of priming cells for a treatment. For example, the cleavage of the chondroitin sulfate proteoglycans allows for better infiltration of therapeutics in the extracellular matrix. This cleavage is beneficial for cancer cells. Cleavage of the proteoglycans allows cancer therapeutics to more efficiently spread through the tumor extracellular matrix and attack the cancer cells.

2. Methods of Treating Cancer

Disclosed are methods of treating cancer by administering to a subject an effective amount of a composition comprising a mutant Chondroitinase ABC I nucleic acid sequence or a mutant Chondroitinase ABC I protein. Mutant Chase ABC can be used to treat cancer because it can cleave chondroitin sulfate glucosaminoglycans. The cleavage of the chondroitin sulfate glucosaminoglycans allows for infiltration of therapeutics into the tumor extracellular matrix.

Disclosed are methods of treating cancer comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein.

The composition comprising a nucleic acid sequence that comprises a mutant Chondroitinase ABC I nucleic acid sequence can further comprise secretion signal sequence. For example, the secretion signal sequence can be an Igκ sequence, BAI1 sequence, MMP sequence or IgG sequence. The secretion signals can allow for effective secretion of the mutant Chondroitinase ABC I protein that is encoded by the nucleic acid sequence.

The compositions used in the methods to treat cancer can comprise a vector. In some aspects, the composition can comprise a vector wherein the vector comprises a mutant Chondroitinase ABC I nucleic acid sequence that encodes a mutant Chondroitinase ABC I protein. The vector can be a viral or nonviral vector. In some aspects, the vector can be a viral vector. For example, the viral vector can be an oncolytic viral vector. Administering an oncolytic virus can be beneficial because oncolytic viruses target cancer cells. Thus, an oncolytic virus that comprises a nucleic acid sequence wherein the nucleic acid sequence comprises a mutant Chase ABC nucleic acid sequence that encodes a mutant Chase ABC protein can infect cancer cells and the mutant Chase ABC protein can be secreted from the cancer cells and cleave chondroitin sulphate proteoglycans. The cleavage of chondroitin sulphate proteoglycans allows for better infiltration of the oncolytic virus which can ultimately lead to the killing of more cancer cells. The cleavage also allows for better infiltration of cancer therapeutics. Therefore, administering Chase ABC, either as a nucleic acid sequence or protein, can provide better infiltration of cancer therapeutics.

Disclosed are methods of treating cancer comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises the nucleic acid sequence of SEQ ID NO:2.

Disclosed are methods of treating cancer comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises a sequence capable of encoding a mutant Chase ABC protein comprising the amino acid sequence of SEQ ID NO:6 or SEQ ID NO:5.

In some aspects, the methods of treating cancer comprising administering to a subject an effective amount of a composition comprising a nucleic acid sequence, wherein the nucleic acid sequence comprises a mutant Chondroitinase ABC I nucleic acid sequence that encodes a mutant Chondroitinase ABC I protein, wherein the mutant Chondroitinase ABC I nucleic acid sequence comprises a sequence capable of encoding a mutant Chondroitinase ABC I protein that comprises at least six mutations compared to wild-type Chondroitinase ABC I. For example, the mutant Chondroitinase ABC I protein can have at least six mutations compared to the amino acid sequence of SEQ ID NO:8.

Disclosed are methods of treating cancer comprising administering to a subject an effective amount of a composition comprising a mutant Chase ABC protein.

The mutant Chase ABC protein present in the compositions used in these methods can comprise at least six mutations compared to wild-type Chase ABC. In some aspects, the mutant Chase ABC protein can comprise the amino acid sequence of SEQ ID NO:6. In some aspects, the mutant Chase ABC protein can comprise the amino acid sequence of SEQ ID NO:5. In some aspects, the mutant Chase ABC protein can further comprise at least three codon optimized amino acids. The codon optimized amino acids can be leucine residues. In some aspects, the leucine residues can be at least at positions 338, 515 and 518 of SEQ ID NO:5, which correspond to positions 342, 519 and 522 of SEQ ID NO:8.

In some aspects, the methods of treating cancer with a composition comprising a nucleic acid sequence, wherein the nucleic acid sequence comprises a mutant Chase ABC nucleic acid sequence that encodes a mutant Chase ABC protein and the methods of treating cancer with a composition comprising a mutant Chase ABC protein can further include administering a cancer therapeutic. The cancer therapeutic can be a chemotherapeutic agent, an anti-inflammatory agent, an immunotherapeutic or an oncolytic virus.

Examples of chemotherapeutic agents include, but are not limited to, adrenocorticosteroids, such as prednisone, dexamethasone or decadron; altretamine (hexalen, hexamethylmelamine (HMM)); amifostine (ethyol); aminoglutethimide (cytadren); amsacrine (M-AMSA); anastrozole (arimidex); androgens, such as testosterone; asparaginase (elspar); bacillus calmette-gurin; bicalutamide (casodex); bleomycin (blenoxane); busulfan (myleran); carboplatin (paraplatin); carmustine (BCNU, BiCNU); chlorambucil (leukeran); chlorodeoxyadenosine (2-CDA, cladribine, leustatin); cisplatin (platinol); cytosine arabinoside (cytarabine); dacarbazine (DTIC); dactinomycin (actinomycin-D, cosmegen); daunorubicin (cerubidine); docetaxel (taxotere); doxorubicin (adriomycin); epirubicin; estramustine (emcyt); estrogens, such as diethylstilbestrol (DES); etopside (VP-16, VePesid, etopophos); fludarabine (fludara); flutamide (eulexin); 5-FUDR (floxuridine); 5-fluorouracil (5-FU); gemcitabine (gemzar); goserelin (zodalex); herceptin (trastuzumab); hydroxyurea (hydrea); idarubicin (idamycin); ifosfamide; IL-2 (proleukin, aldesleukin); interferon alpha (intron A, roferon A); irinotecan (camptosar); leuprolide (lupron); levamisole (ergamisole); lomustine (CCNU); mechlorathamine (mustargen, nitrogen mustard); melphalan (alkeran); mercaptopurine (purinethol, 6-MP); methotrexate (mexate); mitomycin-C (mutamucin); mitoxantrone (novantrone); octreotide (sandostatin); pentostatin (2-deoxycoformycin, nipent); plicamycin (mithramycin, mithracin); prorocarbazine (matulane); streptozocin; tamoxifen (nolvadex); taxol (paclitaxel); teniposide (vumon, VM-26); thiotepa; topotecan (hycamtin); tretinoin (vesanoid, all-trans retinoic acid); vinblastine (valban); vincristine (oncovin) and vinorelbine (navelbine). Suitably, the further chemotherapeutic agent is selected from taxanes such as Taxol, Paclitaxel or Docetaxel.

The mutant Chase ABC protein of the disclosed methods can increase the spread of an oncolytic virus or allow for better infiltration of other cancer therapeutics.

3. Methods of Treating Central Nervous System Injury

Disclosed are methods of treating central nervous system injury by administering to a subject an effective amount of a composition comprising a mutant Chondroitinase ABC I nucleic acid sequence or a mutant Chase ABC protein. Chondroitin sulphate proteoglycans can inhibit axon growth and therefore the presence of a mutant chondroitinase ABC I protein that can cleave these proteoglycans can lead to axon regeneration.

Disclosed are methods of treating central nervous system injury comprising administering to a subject an effective amount of a composition comprising a nucleic acid sequence, wherein the nucleic acid sequence comprises a mutant Chase ABC nucleic acid sequence that encodes a mutant Chase ABC protein.

The composition comprising a nucleic acid sequence that comprises a mutant Chase ABC nucleic acid sequence can further comprise secretion signal sequence. For example, the secretion signal sequence can be an Igκ sequence, BAI1 sequence, MMP sequence or IgG sequence. The secretion signals can allow for effective secretion of the mutant Chase ABC protein that is encoded by the nucleic acid sequence.

The compositions used in the methods to treat central nervous system injury can comprise a vector. In some aspects, the composition can comprise a vector wherein the vector comprises a mutant Chase ABC nucleic acid sequence that encodes a mutant Chase ABC protein. The vector can be a viral or nonviral vector. The vector can be manipulated to target certain cell types or to specifically target the central nervous system.

Disclosed are methods of treating central nervous system injury comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises the nucleic acid sequence of SEQ ID NO:2.

Disclosed are methods of treating central nervous system injury comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises a sequence capable of encoding a mutant Chase ABC protein comprising the amino acid sequence of SEQ ID NO:6 or SEQ ID NO:5.

In some aspects, the methods of treating central nervous system injury comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises a sequence capable of encoding a mutant Chase ABC protein that comprises at least six mutations compared to wild-type Chase ABC. For example, the mutant Chase ABC protein can have at least six mutations compared to the amino acid sequence of SEQ ID NO:8.

Disclosed are methods of treating central nervous system injury comprising administering to a subject an effective amount of a composition comprising a mutant Chase ABC protein.

The mutant Chase ABC protein present in the compositions used in these methods can comprise at least six mutations compared to wild-type Chase ABC. In some aspects, the mutant Chase ABC protein can comprise the amino acid sequence of SEQ ID NO:6. In some aspects, the mutant Chase ABC protein can comprise the amino acid sequence of SEQ ID NO:5. In some aspects, the mutant Chase ABC protein can further comprise at least three codon optimized amino acids. The codon optimized amino acids can be leucine residues. In some aspects, the leucine residues can be at least at positions 338, 515 and 518 of SEQ ID NO:5, which correspond to positions 342, 519 and 522 of SEQ ID NO:8.

In some aspects, the methods of treating central nervous system injury with a composition comprising a nucleic acid sequence, wherein the nucleic acid sequence comprises a mutant Chase ABC nucleic acid sequence that encodes a mutant Chase ABC protein and the methods of treating central nervous system injury with a composition comprising a mutant Chase ABC protein can further include administering a second therapeutic. The second therapeutic can be an anti-inflammatory agent, an immunotherapeutic or a gene therapeutic.

The methods of treating central nervous system injury can occur when the central nervous system injury is a spinal cord injury or any other central nervous system injury that requires neural regeneration. In addition to central nervous system injuries, conditions such as ischemia and inflammatory disorders can be treated with a composition comprising a nucleic acid sequence, wherein the nucleic acid sequence comprises a mutant Chase ABC nucleic acid sequence that encodes a mutant Chase ABC protein. The mutant Chase ABC can be used in combination with known agents that treat the particular disorder.

Following a spinal cord injury, specialized glial cells that normally support neurons can accumulate at the injury site and deposit scar tissue. Scar tissue is a major obstacle to spinal cord repair because it creates a physical and biochemical blockage that neurons must grow through. Molecules that inhibit neuronal growth are concentrated in the scar. Chase ABC has been shown to be an inhibitor to scars. Chase can cleave the chondroitin sulfate proteoglycans in the scar and allow for damaged neurons to regrow. Thus, the disclosed mutant Chase ABC nucleic acids and proteins can be effective at treating central nervous system injuries.

4. Methods of Increasing the Spread of HSV-1

Disclosed are methods of increasing the spread of HSV-1 comprising administering to a cell a composition comprising a nucleic acid sequence, wherein the nucleic acid sequence comprises a mutant Chase ABC nucleic acid sequence that encodes a mutant Chase ABC protein. The mutant Chase ABC protein can cleave chondroitin sulfate proteoglycan from cell surfaces and allow for HSV-1 to better infiltrate the cells. This is particularly useful when HSV-1 is being used to treat cancer. HSV-1 infects cancer cells and the ability to spread faster or more easily provides a better cancer treatment.

The compositions used in the methods to increasing the spread of HSV-1 can comprise a vector. In some aspects, the composition can comprise a vector wherein the vector comprises a mutant Chase ABC nucleic acid sequence that encodes a mutant Chase ABC protein. The vector can be a viral or nonviral vector. In some aspects, the vector can be a viral vector. For example, the viral vector can be an oncolytic viral vector. Administering an oncolytic virus can be beneficial because oncolytic viruses target cancer cells. Thus, an oncolytic virus that comprises a nucleotide sequence wherein the nucleic acid sequence comprises a mutant Chase ABC nucleic acid sequence that encodes a mutant Chase ABC protein can infect cancer cells and the mutant Chase ABC protein can be secreted from the cancer cells and cleave chondroitin sulphate proteoglycans. The cleavage of chondroitin sulphate proteoglycans allows for better infiltration of the oncolytic virus which can ultimately lead to the killing of more cancer cells.

Disclosed are methods of increasing the spread of HSV-1 comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises the nucleic acid sequence of SEQ ID NO:2.

Disclosed are methods of increasing the spread of HSV-1 comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises a sequence capable of encoding a mutant Chase ABC protein comprising the amino acid sequence of SEQ ID NO:6 or SEQ ID NO:5.

In some aspects, the methods of increasing the spread of HSV-1 comprising administering to a subject an effective amount of a composition comprising a nucleic acid sequence, wherein the nucleic acid sequence comprises a mutant Chase ABC nucleic acid sequence that encodes a mutant Chase ABC protein, wherein the mutant Chase ABC nucleic acid sequence comprises a sequence capable of encoding a mutant Chase ABC protein that comprises at least six mutations compared to wild-type Chase ABC. For example, the mutant Chase ABC protein can have at least six mutations compared to the amino acid sequence of SEQ ID NO:8.

Disclosed are methods of increasing the spread of HSV-1 comprising administering to a subject an effective amount of a composition comprising a mutant Chase ABC protein.

The mutant Chase ABC protein present in the compositions used in these methods can comprise at least six mutations compared to wild-type Chase ABC. In some aspects, the mutant Chase ABC protein can comprise the amino acid sequence of SEQ ID NO:6. In some aspects, the mutant Chase ABC protein can comprise the amino acid sequence of SEQ ID NO:5. In some aspects, the mutant Chase ABC protein can further comprise at least three codon optimized amino acids. The codon optimized amino acids can be leucine residues. In some aspects, the leucine residues can be at least at positions 338, 515 and 518 of SEQ ID NO:5, which correspond to positions 342, 519 and 522 of SEQ ID NO:8.

The mutant Chase ABC protein of the disclosed methods can increase the spread of an oncolytic virus or allow for better infiltration of other therapeutics.

Administering to a cell can include direct administration to a cell or indirectly administration to a cell. Indirect administration to a cell includes administering to a subject wherein the cell is inside of the subject.

### D. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits comprising a vector comprising a nucleic acid comprising the nucleic acid sequence of SEQ ID NO:2. The kits also can include, but is not limited to, the isolated mutant Chondroitinase ABC I proteins, isolated mutant Chondroitinase ABC I nucleic acid sequences, as well as cells or a cell line comprising the disclosed vectors or isolated mutant Chondroitinase ABC I nucleic acid sequences.

### E. Administration

The compositions can also be administered in vivo in a pharmaceutically acceptable carrier. By “pharmaceutically acceptable” is meant a material that is not biologically or otherwise undesirable, i.e., the material can be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions can be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

1. Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers can be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions can include carriers, thickeners, diluents, buffers, preservatives, surface active agents and the like in addition to the molecule of choice. Pharmaceutical compositions can also include one or more active ingredients such as antimicrobial agents, antiinflammatory agents, anesthetics, and the like.

The pharmaceutical composition can be administered in a number of ways depending on whether local or systemic treatment is desired, and on the area to be treated. Administration can be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives can also be present such as, for example, antimicrobials, anti-oxidants, chelating agents, and inert gases and the like.

Formulations for topical administration can include ointments, lotions, creams, gels, drops, suppositories, sprays, liquids and powders. Conventional pharmaceutical carriers, aqueous, powder or oily bases, thickeners and the like can be necessary or desirable.

Compositions for oral administration include powders or granules, suspensions or solutions in water or non-aqueous media, capsules, sachets, or tablets. Thickeners, flavorings, diluents, emulsifiers, dispersing aids or binders can be desirable.

Some of the compositions can potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### EXAMPLES

The following examples are put forth so as to provide those of ordinary skill in the art with a complete disclosure and description of how the compounds, compositions, and methods claimed herein are used and evaluated and are intended to be purely exemplary of the invention and are not intended to limit the scope of what the inventors regard as their invention. However, those of skill in the art should, in light of the present disclosure, appreciate that many changes can be made in the specific aspects which are disclosed and still obtain a like or similar result without departing from the spirit and scope of the invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### F. Example 1

### Chondroitinase ABC I Mutagenesis

Bacteria Proteus vulgaris, #6896 (NCTC #4636) was purChased from ATCC. Bacterial DNA was extracted using DNeasy Blood and Tissue Kit according to the manufacturer's protocol for gram negative bacteria (#69504, Qiagen Sciences, Maryland 20874, USA). The DNA was used as the template for PCR to generate a cDNA of Chondroitinase ABC I lacking an N-terminal signal sequence. The encoded sequence of the gene for Proteus vulgaris (Ryan, M J et al., Patent WO 94/25567) was confirmed.

For effective secretion of Chondroitinase ABC I double stranded oligo of human Ig κ-chain leader sequence flanked with NheI and EcoRI digestion sites was generated and was cloned to pcDNA3.1D/V5/His TOPO (Invitrogen). cDNA of Chondroitinase ABC I was ligated to pcDNA3.1D/V5/His TOPO followed Ig κ leader sequence. Cloned Chondroitinase ABC I sequence with human Ig κ leader sequence served as the template for site-directed mutagenesis.

Primers G1, G2, G3 and G5 (Muir, E M et al., 2010, J Biotechnol, 145(2) 103-110) were used to mutate the selected N-glycosylation sites in Chondroitinase ABC I cDNA using QuickChange Lightning Multi Site-Directed Mutagenesis Kit (#200514, Agilent Technologies, 11011 North Torrey Pines Road, La Jolla, Calif. 92037).

The following sites of protein Chondroitinase ABC I have been mutated: N282Q; N338Q; N345Q, L342 codon improved; S517A, L519 and L522 codons improved; N675Q.

The highly active clone Y133 had the following mutations: N282Q; N345Q; S517A; N751Q; N675Q.

After mutations of five glycosylation sites primer: 3′-GTTCCACTGGTGACACCAGCAATCCTG-5′ was used to delete EcoRI site between Ig κ sequence and Chondroitinase ABC I sequence. The final sequence of Chondroitinase ABC I (Chase I) used for the generation of OV Chase M is SEQ ID NO:2.

### G. Example 2

### Testing for Secretion of Active Chondroitinase ABC I

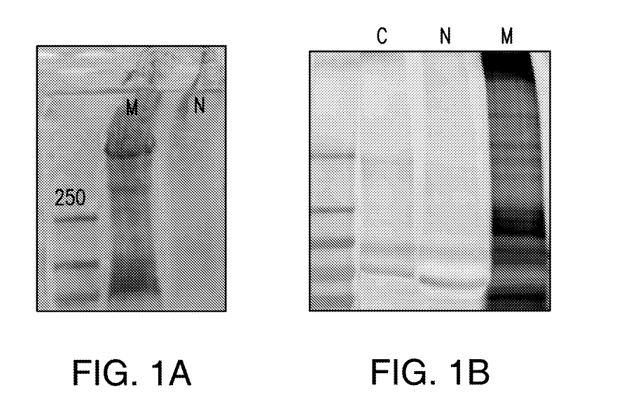

COS-7 cells were transfected with cDNAs: cDNA Chase M (mutation) or cDNA Chase N (no mutation) using FuGENE 6 Transfection reagent (Roche Applied Science Inc). After 24 h concentrated medium from U87ΔEGFR (source of CSPG) was added to transfected COS-7 cells. Forty eight hours later the medium from COS-7 cells was collected, concentrated 120 times and was probed for anti-Chondroitin-4-sulfate antibody (BE 123 clone, Millipore Corp, 28820 Single Oak Drive, Temecula, Calif.). The antibody recognizes a stub left after digestion of CS by Chondroitinase ABC from CSPGs. As shown in FIG. 1A, Western Blot analysis showed multiple bands only in the samples transfected with mutated Chondroitinase ABC I cDNA (M).

cDNA Chondroitinase ABC I (normal or mutant) was utilized to generate two oncolytic HSV-1 based viruses, OV Chase N and OV Chase M. Secretion of active Chondroitinase ABC I from viruses was tested on COS-7 cells. COS-7 cells were infected with control virus, OV Chase N, or OV Chase M, MOI-3, for 1 hour. The viruses were rinsed off, and concentrated medium from U87ΔEGFR cells was added to the cells. After 15 hours, the cell medium was collected, concentrated and was probed with anti-Chondroitin-4-sulfate antibody. As shown in FIG. 1B, only OV Chase M virus produced stub immunoreactivity.

### H. Example 3

### Testing for Secretion of Active Chondroitinase ABC I In Vivo

GBM 9 cells were electroporated with pcDNA3.1D/ChaseM or pcDNA 3.1D/LacZ and injected into the striatum of athymic mice (300,000 cells/mouse). Seven days later tumors were removed from the mice, fixed in 4% PFA, cut and labeled with anti-chondroitin-4-sulfate antibody.

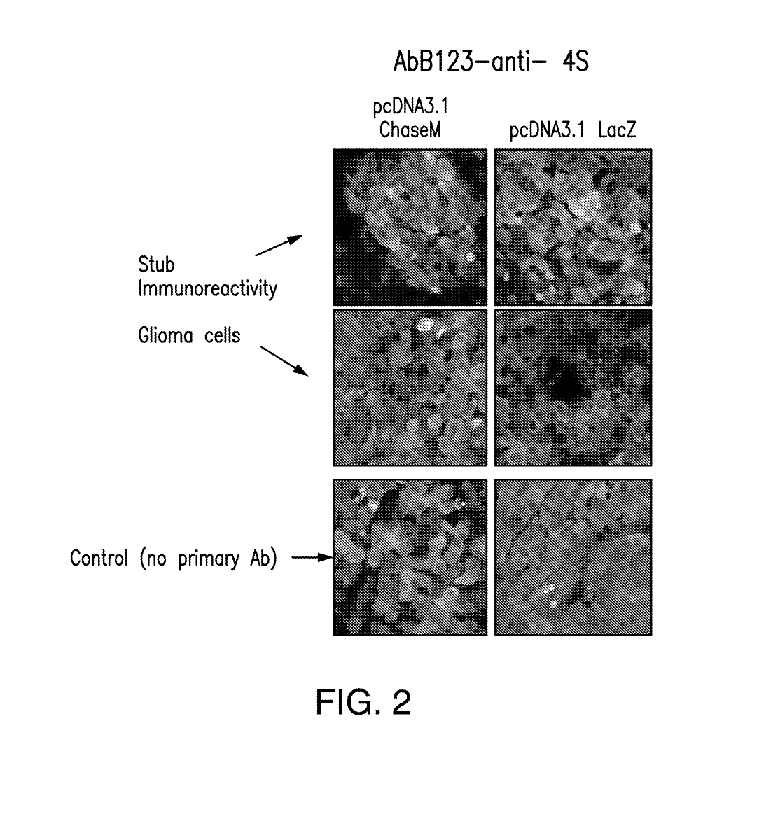

As shown in FIG. 2, anti-chondroitin-4-sulfate immunoreactivity was observed only in the GBM 9 cells originated from the cells transfected with pcDNA3.1D/ChaseM.

### I. [0030] Example 4

### Verification of Insertion of Chondroitinase ABC I cDNA into Viruses

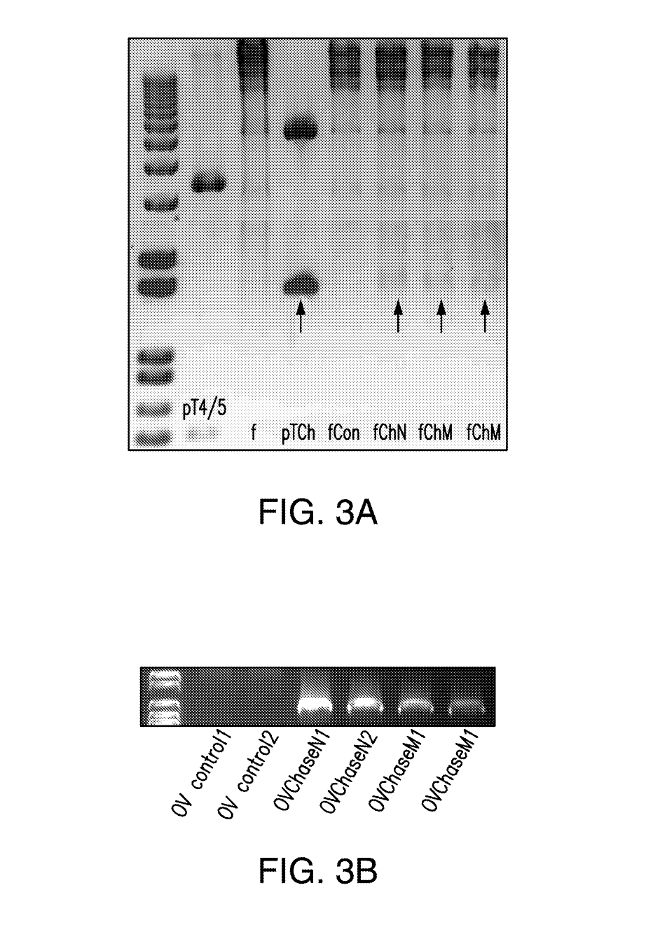

Verification of insertion of Chondroitinase ABC I was performed at the nucleic acid level. Protein was isolated from DH10B cells transfected with pTransfer shuttle plasmid (pT4/5), BAC plasmid (f), shuttle plasmid with Chase cDNA (pTCh), BAC co-integrant without Chase cDNA (fCon), BAC co-integrant with ChaseN cDNA (fChN), or BAC co-integrant with ChaseM cDNA (fChM). Plasmid DNAs were digested with Hind III restriction enzyme and the restriction pattern was visualized on a gel, as shown in FIG. 3A. PCR verification of insertion of Chondroitinase ABC I cDNA into oncolytic HSV vectors was also performed, as shown in FIG. 3B.

### J. Example 5

### LN229 Spheres Treated with Different HSVQ Viruses and Embedded into Collagen

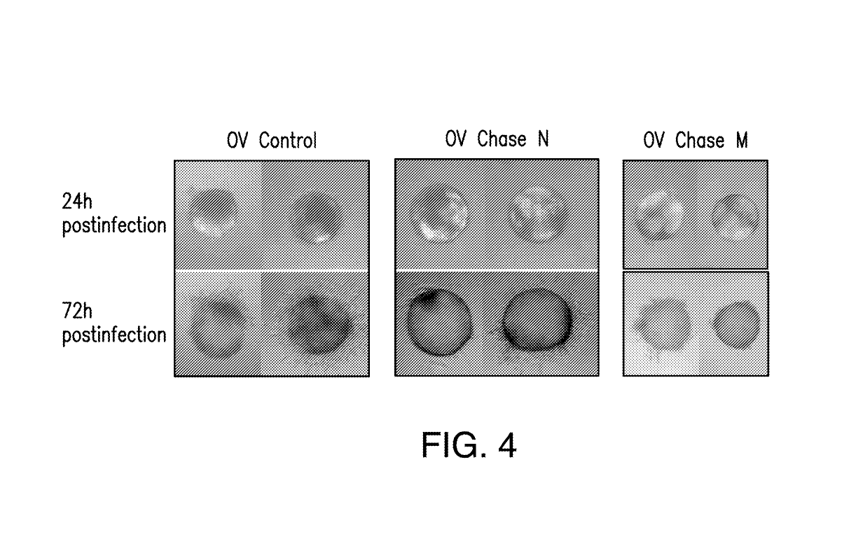

LN229 cells were cultured as a hanging drops (2,000 cells/20 μl) for 3 days, infected with OVs (10⁴ pfu/sphere) for 1 hour, and then embedded into a collagen solution. Pictures were taken after 1 and 3 days. OV Chase M showed better spread of OV particles than OV Control and OV Chase N in this model. As shown in FIG. 4.

### K. Example 6

### Increased Spread of OV ChaseM Compare to OV Control after 24 Hours in Glioma Neurosphere Models (GBM 2 and GBM 44)

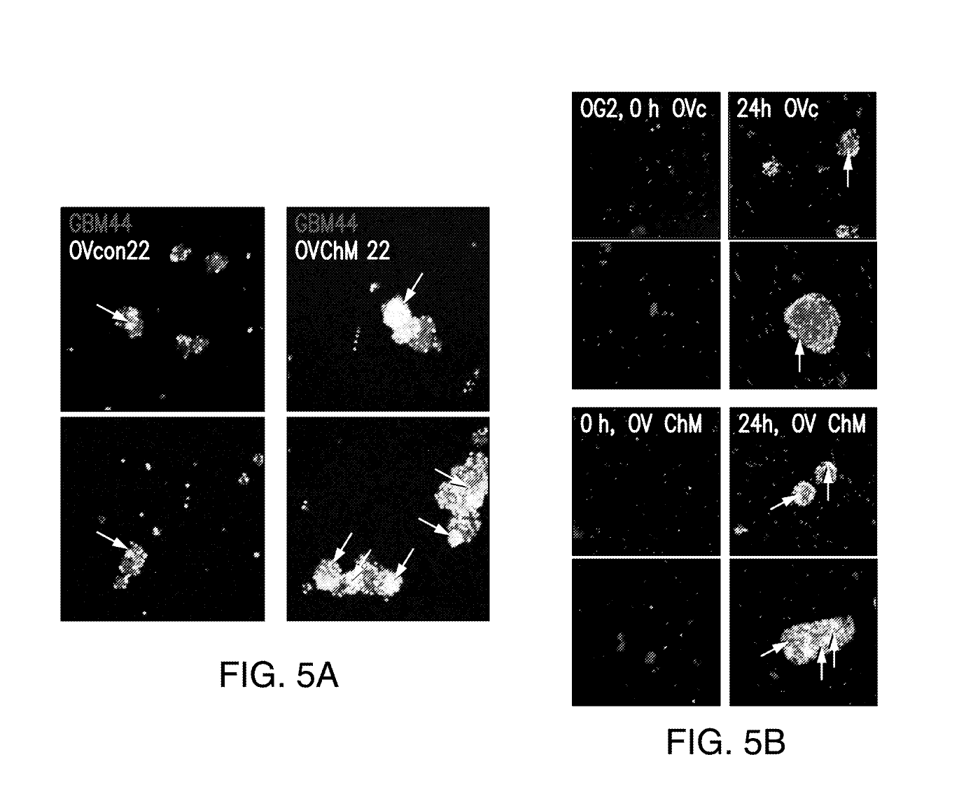

7×10⁵ GBM neurospheres were dissociated into single cell suspension, labeled with Red Tracker according the instructions for #C34553 (Invitrogen), and cultured in 25 cm² flasks. The next day, 5×10⁵ single GBM cells in suspension were inoculated with OV Control or OV Chase M at MOI-0.2. After 3 hours the infected cells were spun to remove the virus from medium and transferred into the flasks with Tracker-labeled glioma aggregates. The images of the spread of viral GFP into the red cells from aggregates were taken under a confocal microscope after 24 hours, to allow 1 round of viral replication. The microscopic image were used to determine the spread of the virus. GBM 44 cells were used in FIG. 5A and OG 2 cells were used in FIG. 5B were labeled with a red tracker. Virus-infected cells were labeled with green. GBM cells that were infected with virus created a yellow overlay signal denoted by arrows in FIG. 5. After 24 hours, it was apparent that OV Chase M spread more than OV control.

### L. Example 7

### In Vivo Intracranial Human Glioma from Primary Glioma Spheroid Culture

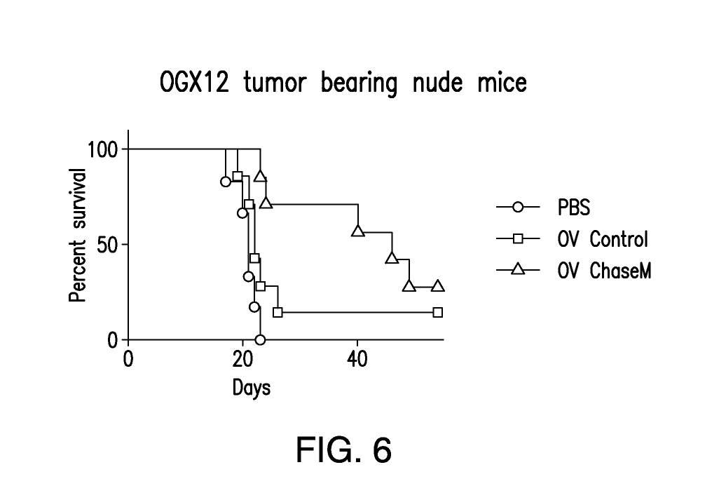

Glioma spheroid culture were created from primary intracranial human gliomas (OG9 and OG X12) and cultured in defined medium with growth factors. The spheroid cultures were implanted (100,000 cells) into the brains of nude mice. Eight days after the implantation of tumors, 2×10⁵ pfu of OV Chase M or OV control were inoculated into the tumor. PBS treatment was a control for the viral treatment. Percent survival was followed for a maximum of 45 days following administration of PBS, OV control, or OV Chase M. In both models, survival was significantly improved by inoculation with OV Chase M compared to the animals that received PBS or OV control, regardless of whether the animals were implanted with OG9 glioma or OG X12 glioma (FIG. 6).

### M. Example 8

### Aggregation of Human Glioma Neurospheres after Transfection with Chase cDNA Vectors

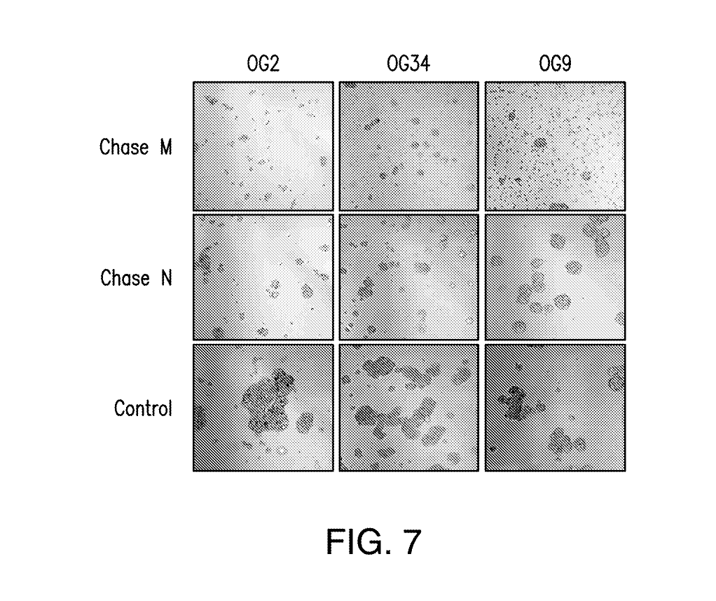

pcDNA3.1 vector (control), pcDNA3.1Chase N vector, or pcDNA3.1Chase M vector was electroporated into 5-2.5×10⁶ single cells in suspension from OG2, OG9 or OG34 cultures using Amaxa Nucleofactor Kit in Amaxa Transfection Mashine (Lonza). Next the cells were cultured in the flasks in Neurobasal medium for 48 hours and the images were taken in 4-5 different areas under the microscope. FIG. 7 shows that regardless of cell type, cells transfected with Chase M had reduced aggregation compared to cells transfected with Chase N or control vector.

### N. Example 9

### Increased Synergy to TMZ (Temozolamide) after Chase M Transfection in Glioma Neurospheres OG9

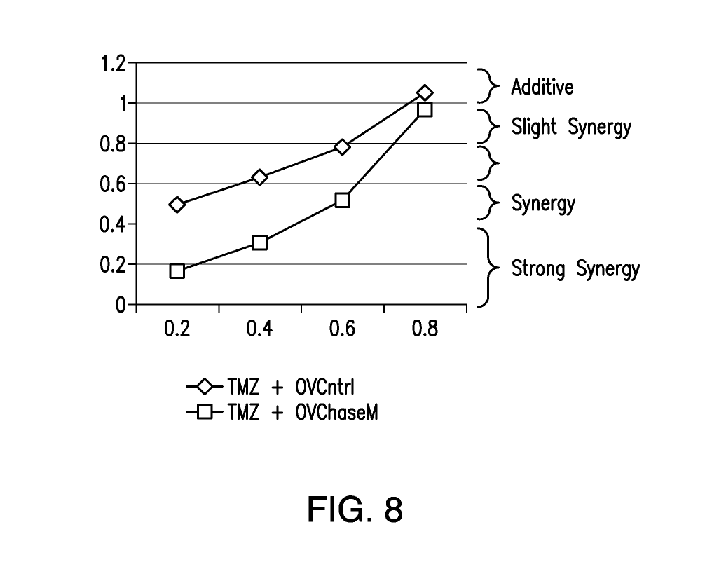

OG9 neurospheres were seeded into 96-well dish as a single cell suspension, 5000 cells/well in neurosphere medium, in triplicate. After 2 hours the cells were treated with TMZ alone, or with OV Chase M alone or with OV Control alone. Four hours later, TMZ was added to some wells with cells treated with oncolytic viruses. After 5 days, MTT assays were conducted to determine viable cell number. Synergy of the administration of TMZ/Oncolytic virus was quantified using the Chou-Talay method and is shown in FIG. 8.

### O. Example 10

### Caspase 3/7 Activity in Human Glioma Neurospheres after TMZ Treatment

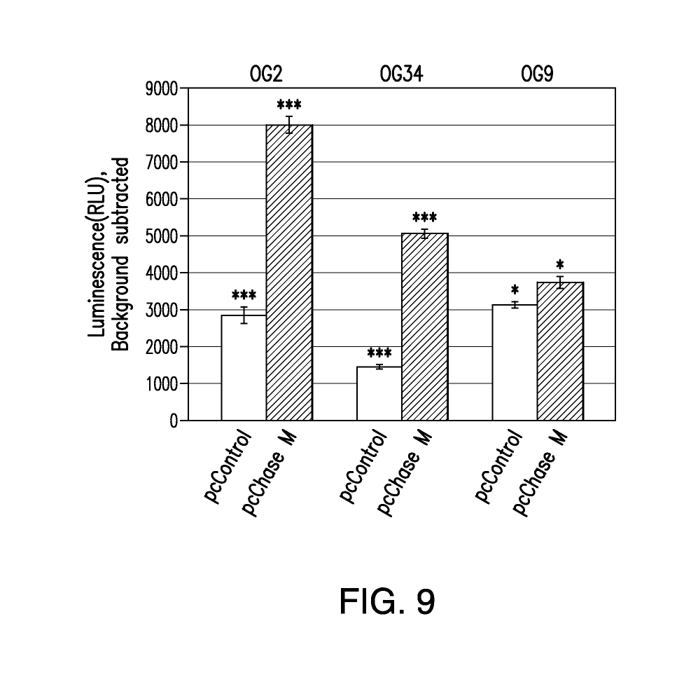

2×10⁶ single cells from human OG2, OG34, and OG9 cells were electroporated with pcDNA3.1 vector or with pcDNA3.1ChaseM (5 μg), and cultured in Neurobasal medium in the flasks. After 48 hours, 300 μM of TMZ were added to the flacks for 3 days. 10,000 cells in 100 μl for each condition were plated into 96-well culture dishes in triplicates. Caspase-Glo 3/7 Assay System (Promega) was used to measure the caspase-3/7 activities. As shown in FIG. 9, transfection with Chase M significantly increased caspase activity.

### P. Example 11

### Sensitivity to TRAIL (TNF-Related Apoptosis-Inducing Ligand) in Glioma Cells after Chase Treatment

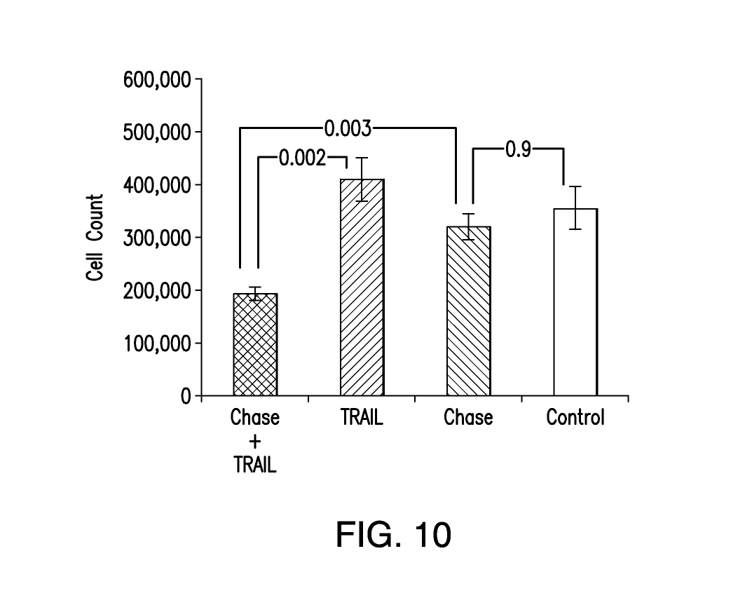

U87ΔEGFR glioma cells were cultured in 6-well low adherent culture dishes (2×10⁵/well) while slow shaking on a stirring plate in order to aggregate the cells. Purified enzyme Chondroitinase ABC I (Seikagaku Inc, Japan) at the concentration 0.033u/well was added for 24 hours. The next day, TRAIL (R&D systems), 15 ng per well, was added with Chase ABC I (0.033u/well). After 24 hours, the amount of glioma cells was counted by Trypan Blue exclusion. The experiment was performed in triplicate, and results are shown in FIG. 10.

### Q. Example 12

### Oncolytic Viruses and Chondroitinase

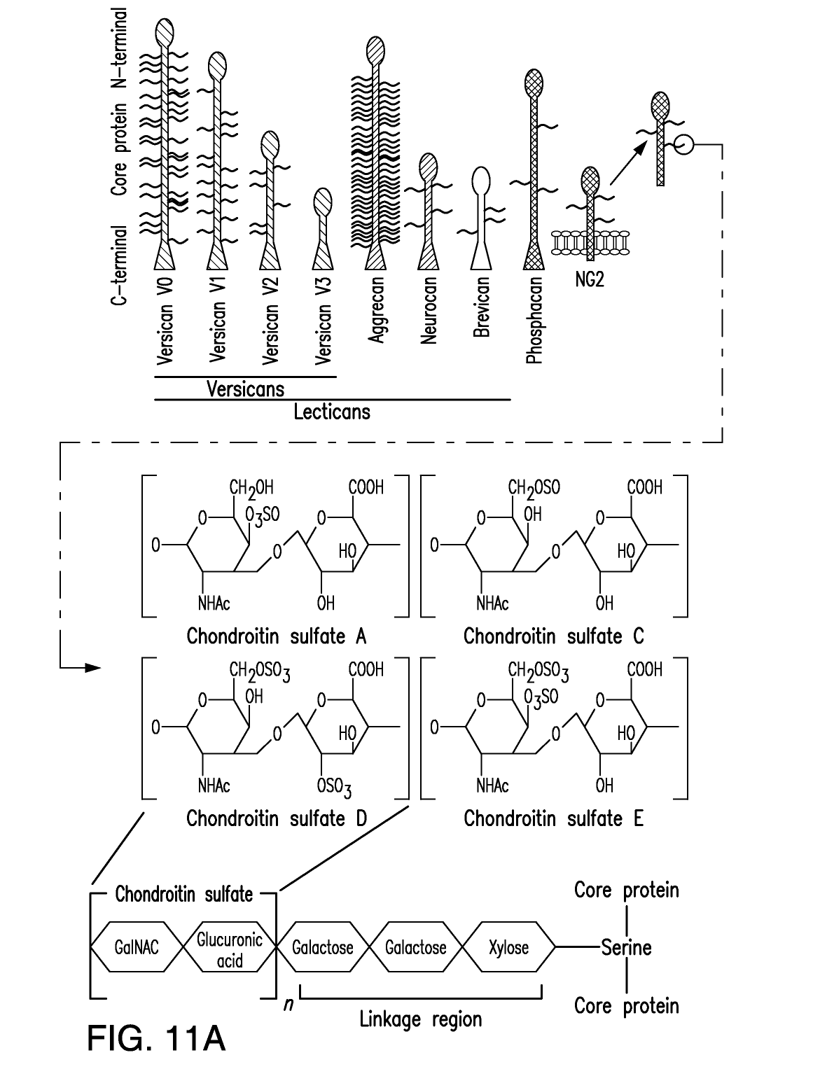

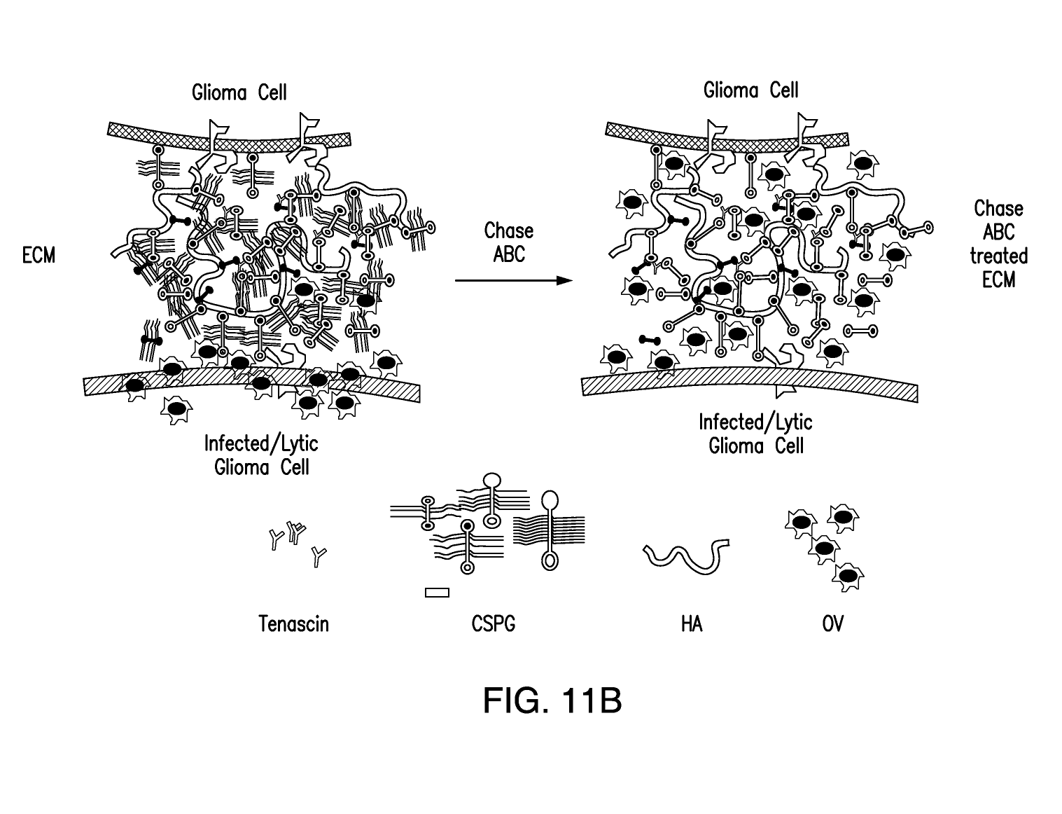

Oncolytic viruses are viruses that have tumor-specific replication, which gives this type of virus great potential for use as anti-neoplastic agents. However, current limitations of the technology include difficulty in achieving an effective amount of intra-tumoral spread. As shown in FIG. 11, inoculation with a virus may only result in a single viral plaque which is only capable of damaging a small area of the tumor.

The difficulty in achieving effective intra-tumoral spread stems from the dense extracellular matrix (ECM) produced by the tumor cells. This ECM blocks the spread of oncolytic virus between lytic cells and uninfected cells. However, Chondroitinases are bacterial enzymes that can cleave a variety of chondroitin sulfate glucosaminoglycans (CS GAGs) covalently attached to the core of CSPGs to tetrasaccharides and dissaccarides without altering the core protein structure, as shown in FIG. 11A. These CS GAGs constitute a major component of the ECM, therefore cleavage of CS GAGs allows for more efficient and effective spread of oncolytic virus, as shown in FIG. 11B.

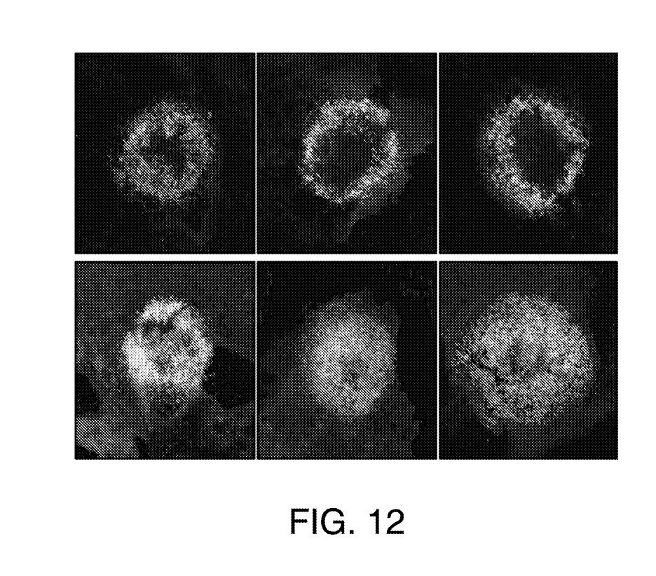

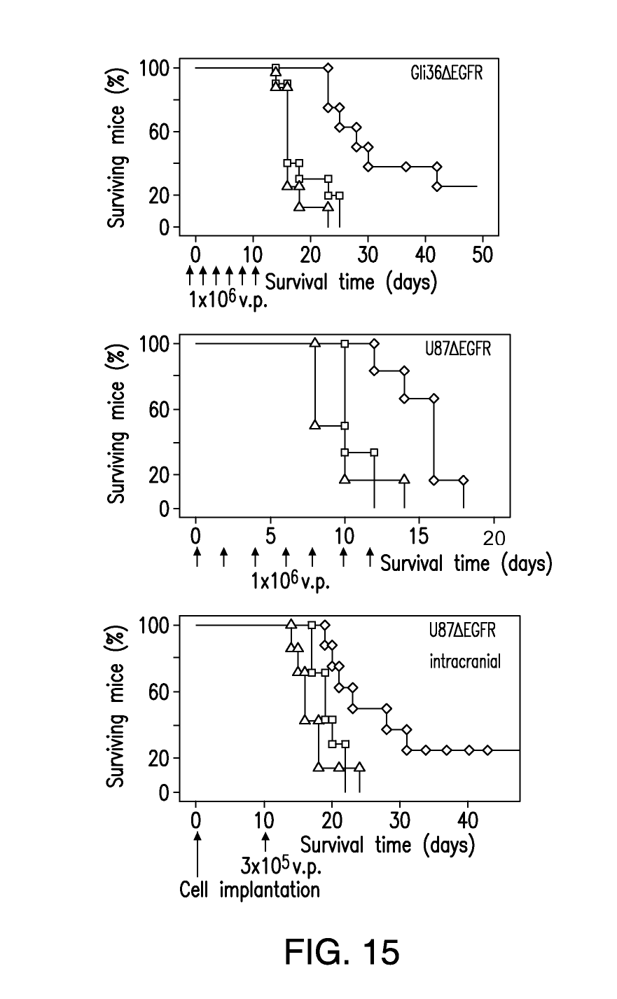

As a proof of concept, cultures inoculated with OV were also treated with purified Chase ABC enzyme, as shown in FIG. 12. Briefly U87delta EGFR glioma cell line spheroids on Organotypic brain slice cultures were treated with or without purified Chase and then infected with OV. Chase ABC treatment resulted in an increase in viral spread. Moreover, animal models inoculated with OV expressing wild type Chase ABC showed improvement in anti-tumor efficacy, as shown in FIG. 15. Gli36ΔEGFR mice were inoculated with 1×10⁶ viral particles on six days (shown by arrows), and percent survival (FIG. 15A) and tumor volume were followed over the course of the study. U87ΔEGFR mice were also inoculated with 1×10⁶ viral particles on six days (shown by arrows), and percent survival (FIG. 15B) and tumor volume were followed over the course of the study. U87ΔEGFR mice were also inoculated with 3×10⁵ viral particles once ten days after cell implantation (shown by arrow), and percent survival (FIG. 15C) was followed over the course of the study.

### R. Example 13

### The Tumor Microenvironmental Barrier to Effective Viral Oncolysis

This study shows the different barriers faced by oncolytic viral (OV) therapy in order to enhance treatment for glioma. This study shows the impact of the tumor microenvironment as it relates to its extracellular matrix on the dispersal of oncolytic HSV (oHSV) and optimizes strategies to improve intra-tumoral viral spread thus enhancing anti-tumor efficacy. The tumor's extracellular matrix (ECM) can pose a significant barrier for efficient viral spread, thus limiting virotherapy efficacy. Chondroitin sulfate proteoglycans (CSPG) are one of the major components of glioma ECM. Choindroitinase ABC 1 (Chase ABC) is a bacterial enzyme that can depolymerize this ECM scaffold. Chase ABC mediated digestion of glioma CSPG can enhance oHSV dissemination and efficacy. These studies have uncovered: 1—High expression of CSPG in glioma in humans; 2—Treatment of glioma cells in vitro with Chase ABC does not negatively affect oHSV infection/replication in tumors; 3—Treatment of three dimensional glioma spheroids (GS) grown in an organotypic brain slice model with Chase ABC enhances viral spread throughout the tumor sphere and increases anti-tumor efficacy; 4—Chase ABC does not cause increased tumor cell invasion; and 5—a mutant humanized version of the Chase ABC with better secretion and functionality in mammalian cells. This study examines the mechanism and impact of Chase ABC on oncolysis. Reduced virus replication can contribute to limited oncolytic efficacy of oHSV. Several innovative approaches are currently under investigation including transcriptional retargeting of viruses to drive virulence under the control of tumor-specific promoters. rQNestin34.5 is an oHSV that expresses viral ICP34.5 regulated by glioma-specific nestin promoter in an ICP34.5 deleted viral backbone. This virus has shown excellent antitumor efficacy against glioma. An oHSV expressing the humanized sequence of Chase ABC within the context of rQNestin34.5 can be used. The rQNestin34.5 backbone has been found to be highly effective against models of glioma in vitro and in vivo. The anti-tumor efficacy of the Chase ABC-expressing oHSV in vitro and in vivo, in glioma can be tested. The impact of the Chase ABC-expressing oHSV on tumor immune cell infiltration and responses can also be tested.

The following three things are focused on in this study: 1) the efficacy and mechanism of Chase expressing oHSV in vitro and in vivo; 2) the magnitude and type of tumor micro-environmental changes caused by OVChase expression and its effects on tumor and normal brain; and 3) the impact of Chase ABC-expressing virotherapy on the infiltration and activation status of natural killer (NK) and macrophage cells.

The efficacy and mechanism of Chase expressing oHSV in vitro and in vivo can be determined. Tumor chondroitin sulfated proteoglycans (CSPGs) can limit oHSV efficacy, and expression of Chase ABC within the backbone of rQnestin34.5 (rQnestin34.5 is previously described in Kambara et al. Cancer Res. 65:2832-9, 2005) circumvents this limitation, thus improving oHSV dispersion in the glioma stroma. Both reduced virus replication and spread are reasons for limited success of oncolytic HSV-1 therapy. This study engineers a novel oncolytic virus expressing humanized Chase within rQnestin34.5, a transcriptionally driven oHSV backbone. This will yield an oncolytic oHSV that displays efficient tumor specific replication and also disperses to a high degree within the stroma of malignant glioma.

The magnitude and type of tumor micro-environmental changes caused by OVChase expression and its effects on tumor and normal brain can be characterized. Treatment of glioma CSPG with Chase ABC can change the tumor microenvironment by altering perfusion and vasculature, but does not affect glioma invasion. Understanding how Chase ABC expression alters glioma biology ensures its safety in the context of oHSV-mediated delivery.

The impact of Chase ABC-expressing virotherapy on the infiltration and activation status of natural killer (NK) and macrophage cells can be determined Treatment of glioma CSPG with Chase ABC can alter the infiltration of NK cells and monocytic macrophage and microglial cells within oHSV infected glioma. Elucidating whether immune cell infiltration into the tumor and tumor cell migration are altered by Chase ABC treatment clarifies the impact of Chase ABC on tumor microenvironment.

One of the major barriers for effective drug delivery within the tumor parenchyma is the ubiquitous extracellular matrix (ECM). This matrix forms a complex scaffold that modulates tumor cell proliferation, cell adhesion, and motility. Glioma ECM is based on a scaffold of hyaluronic acid (HA) with associated glycoproteins and proteoglycans, which resemble the composition of the normal brain ECM2. However, the ECM of glioma also includes mesenchymal proteins that are absent in normal brain and that render the matrix of these tumors distinct from the ECM of normal neural tissue and of other solid tumors. Increased expression and extracellular accumulation of ECM reduces the interstitial spaces and increases the internal pressure in the tumor. This leads to an increase in the fractional volume and tortuosity of the extracellular space, which are the major biophysical factors that limit passive molecular diffusion in the tumor tissue and limit the biodistribution of therapeutics, such as OVs.

Proteases have been tested to enhance OV distribution, with demonstrated efficacy in subcutaneous tumor models. However, the expression of proteases and collagenase in the brain can result in toxicity, primarily related to hemorrhages. In a recent study showing improved efficacy of oncolytic HSV-1 after degradation of fibrillar collagen, the authors remarked that: “intratumoral hemorrhages occurred in many tumors treated with collagenase. They further speculated that “It is possible that collagenase treatment of tumors may increase the risk of metastasis”. More recently co-administration of hyaluronidase with adenovirus has shown increased viral spread and therapeutic efficacy against subcutaneous tumors in mice. However, the expression of hyaluronidase is known to elicit astrocytic reactivity which may also limit its utility for intracranial glioma.

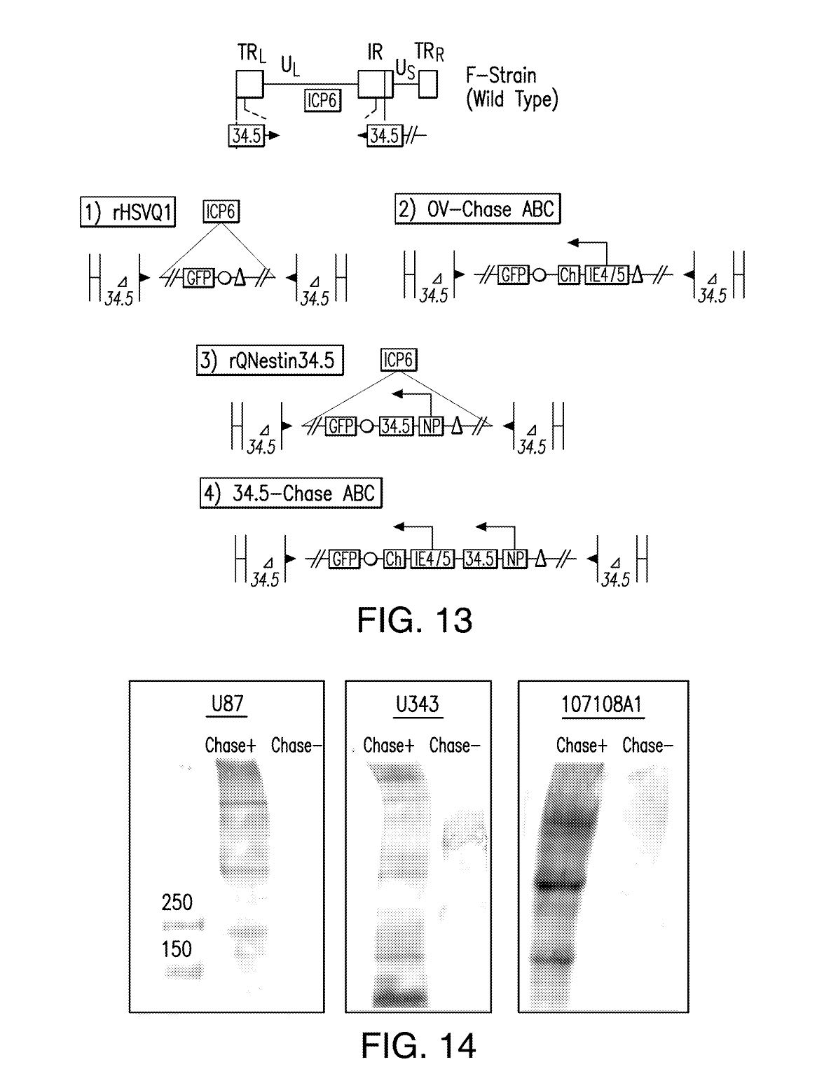

Thus, the use of matrix modulating enzymes to enhance therapy is promising, but needs to be carefully evaluated for safety. On the contrary, repeated injections into rodent brains and spinal cords of bacterially produced and purified Chase ABC has revealed remarkable and, perhaps, surprising absence of toxicity/hemorrhages/glial reactivity. Intracerebral injections of purified Chase ABC did not reveal loss of neurons or alterations of their structure nor reactions of glial cells in brains either immediately or after five months of treatment. Further, a sulfated disaccharide derived from chondroitin sulfate proteoglycan (CSPG) has been shown to protect against neuronal inflammation. Disruption of ECM CSPG in the brain has been shown to improve axonal regeneration in multiple studies and based on its preclinical safety, a Phase III trial to evaluate its efficacy in subjects with spinal degeneration is ongoing. (NCT00634946: clinicaltrials.gov). These published studies thus provide a scientific rationale for the design of an oHSV that can efficiently express and secrete large amounts of Chase ABC, a CSPG modulating enzyme. A matrix modulating enzyme based on Chase ABC can be tolerated better than collagenase or hyaluronidase, while retaining enhanced therapeutic efficacy for tumors. Apart from extracellular barriers, transcriptional retargeting under the control of tumor-specific promoters to drive virulence is a powerful anticancer strategy for oHSV. rQNestin34.5 is an oHSV that expresses viral ICP34.5, regulated by the glioma-specific nestin promoter in an ICP34.5 deleted viral backbone. This virus has shown excellent antitumor efficacy against glioma. In this study, a novel oHSVengineered to also express Chase ABC within the backbone of rQNestin34.5 can be engineered and tested. FIG. 13 shows schematics of the different viruses that can be engineered. For simplicity Chase in this figure refers to humanized Chase ABC. An oHSV expressing a therapeutic transgene in this backbone has also been engineered indicating feasibility of this approach.

HA and the HA-associated chondroitin sulfate proteoglycans (CSPGs) are the key molecules that organize the pericellular ECM scaffold in gliomas. HA as well as several CSPGs (versican, brevican, phosphacan) are highly up-regulated in glioma compared to normal tissue. Increased expression of CSPGs such as brevican, versican, phosphacan and NG2 has been linked to glioma growth, invasion and angiogenesis, and these molecules have been suggested as potential therapeutic targets. Targeting approaches using antibodies (versican31) and RNAi (phosphacan33) have led to impairment of tumor proliferation and invasion. The collective evidence indicates that reduction of CSPGs by enzymatic manipulation can reduce glioma invasion and angiogenesis in vivo.

1. The Efficacy and Mechanism of Chase Expressing oHSV In Vitro and In Vivo

i. Expression of CSPGs in Glioma Cell Lines and Human GBM Specimens.

To confirm the presence of CSPGs in human glioma cell lines and GBM specimens, culture medium was collected from glioma cell lines and also harvested tumor lysates from a freshly excised human GBM specimen (107108A1). Although chondroitin sulfate glycosaminoglycans (GAGs) on CSPG are not usually immuno-reactive, their digestion with Chase ABC generates a specific immuno-epitope on the peptide moiety of the CSPG. This is the standard method for evaluation of CSPG expression in cell lines. The indicated cell conditioned medium or tumor lysate was treated with Chase ABC or vehicle. The digested conditioned medium was then analyzed by western blot using the BE-123 mouse monoclonal antibody. Expression of these stubs after Chase ABC-mediated digestion of CSPGs reveals that CSPGs populate the ECM of glioma cell lines, human brain and human glioma (FIG. 14).

ii. Effect of Chase ABC Treatment on oHSV Spread in Glioma.

As a preliminary result, the effect of Chase ABC treatment on spread by the rQNestin34.5 oHSV was tested in an organotypic glioma model. Briefly, U87ΔEGFR human glioma spheroids (made by the hanging drop method), grown on 300-μm organotypic brain slices from 5-7-day-old mice, were infected with 10⁴ pfus of rQNestin34.5, in the presence/absence of Chase ABC. Spread of oHSV in the spheres was visualized by fluorescent imaging of oHSV encoded GFP in infected cells (n=3/group). There was increased oHSV spread within all three glioma spheroids in the presence of purified Chase ABC (FIG. 12).

iii. Engineering Chase ABC-Expressing OV.

The HSVQuik methodology was employed to engineer a novel oHSV, OV-Chase ABC41. In this construct, deletions of both copies of the endogenous ICP34.5 genes are maintained, similar to rHSVQ1 (see FIG. 13). OV-Chase ABC could infect and replicate as well as rHSVQ1 in cells grown in a monolayer and the functionality of Chase ABC produced by OV-Chase ABC was confirmed by immuno-fluorescent staining for immuno-reactive stubs in spheres treated with OV-Chase ABC compared to cells infected with the control oHSV, rHSVQ17.

iv. Enhanced Anti-Tumor Efficacy of OV-Chase ABC.

The anti-tumor efficacy of OV-Chase ABC compared to rHSVQ1 was tested against subcutaneous and intracranial gliomas in mice (FIG. 15). Briefly, athymic mice with subcutaneous and intracranial human glioma were treated with rHSVQ1 or OV-Chase ABC. Mice bearing subcutaneous tumors were monitored for tumor growth and mice bearing intracranial tumors were monitored for survival (FIG. 15). Collectively, these results indicated that OV-Chase ABC had a significant anti-tumor effect in vivo compared to control rHSVQ1 treatment.

v. Enhanced oHSV Spread in Glioma Cell Spheroids Infected with OV-Chase ABC Compared to Glioma Spheroids Infected with HSVQ.

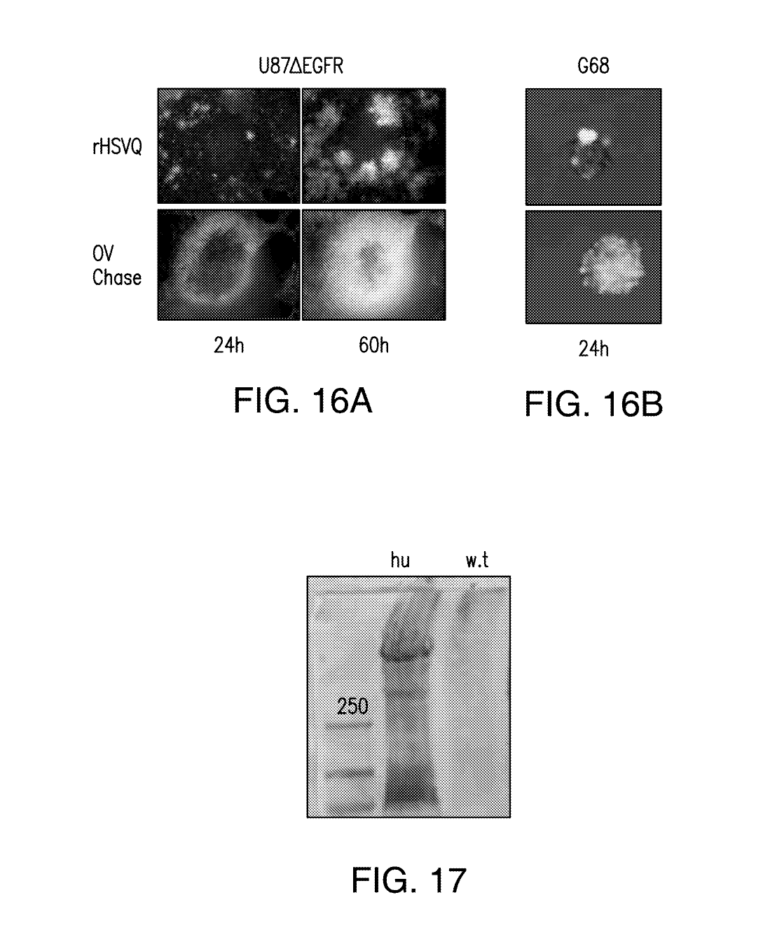

The ability of OV-Chase ABC to spread through glioma spheroids (GS) compared to control HSVQ virus was tested (FIG. 16). Briefly, U87ΔEGFR human glioma spheroids or primary GBM derived neurospheres G68 were grown on organotypic brain slice cultures. Increased spread of oHSV (GFP positive image) to the core of spheres was apparent in both the glioma cell line and the GS infected with OV-Chase ABC, compared to rHSVQ1 (FIG. 16).

These data provide the proof of principle that OV-Chase ABC exhibits improved spread and antitumor efficacy compared to parental rHSVQ1 virus. The bacterial Chase ABC genetic sequence can be humanized to optimize its secretion in mammalian cells and improve its antitumor efficacy. In mammalian cells, secreted proteins contain the consensus Asn-X-Ser/Thr (NXS/T) motifs that are putative sites for N glycosylation. Glycosylation of bacterial enzymes at such sites can impede their activity. Mutational analysis of seven predicted glycosylation sites revealed enhanced activity of a mutant Chase ABC (Y133) harboring N751→Q751, S517→A517, N345→Q345, N675→Q675, AND N836→Q836, mutations. Site directed mutagenesis was used to modify the following amino acids within Chase ABC NXS/T motifs: N751, S517, N345, N675, and N836. The mutations were verified by sequencing. Increased secretion of functional humanized Chase ABC compared to Chase ABC was verified by electroporating the two sequences in mammalian cells. The harvested CM was tested for its ability to digest CSPG present in glioma cell CM (FIG. 17).

vi. Humanized Chase ABC

The sequence of huChase ABC can be used to create 34.5N-huChase ABC. 34.5N-huChase ABC in an oncolytic virus engineered to express the Chase ABC sequence within the rQNestin34.5 backbone. Effective anti-tumor efficacy of utilizing the rQnestin34.5 viral backbone to create second generation more efficacious oncolytic viruses has been shown. FIG. 13 shows the schematic of the different viruses that can be engineered.

In addition, the EGFR retargeted oHSV can be used to add recombinant Chase ABC into the EGFR-retargeted and miRNA regulated oHSV.

vii. Validate the Production of Functional Chase ABC from OV-huChase ABC and 34.5N-huChase ABC.

In addition to human glioma cell lines, primary human tumor derived Neurosphere cultures can be used. Glioma cells (3 glioma cell lines and 3 GSs), can be infected with control viruses (rHSVQ1, or rQnestin34.5) or Chase ABC expressing viruses (OV-huChase ABC and 34.5N-huChase ABC), at MOI=0.01, 0.1, 1 and 10 (n=3/cell line and condition). 24 hours after infection conditioned medium from cells can be harvested, and the development of immuno-reactive stubs on secreted CSPG can be measured by western blot analysis as described in FIG. 13 and FIG. 17. Primary tumor derived spheroids can be infected and stained for the presence of immuno-reactive stubs by immuno-fluorescence.

viii. Effects of Chase ABC Expression on oHSV Replication.

To evaluate the effect of virus encoded Chase ABC expression on viral replication, the number of infectious progeny viruses generated from cells infected with control viruses (rHSVQ1, or rQNestin34.5) or Chase ABC expressing viruses (OV-huChase ABC and 34.5N-huChase ABC) can be measured. Briefly 5×10⁵ glioma cells (three cell lines, and three GSs (G35, 157GBM and GBM30) can be infected with control viruses (rHSVQ1, or rQNestin34.5) or Chase ABC expressing viruses (OV-huChase ABC and 34.5N-huChase ABC) at an MOI of 0, 0.001, 0.01, 0.1, and 1. Yield of progeny viruses can be compared 16 hours after infection (single round of replication) and 48, 72, and 96 hours after infection to compare effect of wt and mutant Chase ABC expression on subsequent rounds of viral replication. The cells and CM can be harvested and the virus can be isolated after 3 freeze thaw cycles. The number of infectious viral particles harvested from each culture can be then estimated by a standard viral titration assay in a 96-well plate with Vero cells.

ix. To Compare the Oncolytic Ability of Chase ABC Expressing Viruses to Control Viruses.

One day before infection, cells (three cell lines and three GSs, and normal human astrocytes (ScienCell Research laboratories)) can be plated in five 96 well plates. On day zero the cells can be infected with control viruses (rHSVQ1, or rQnestin34.5) or Chase ABC expressing viruses (OV-huChase ABC and 34.5N-huChase ABC) at MOI=0, 0.05, 0.1, 0.5, or 1. The plates can be harvested on day 0, 1, 2, 3, 4, 5 after infection, fixed and proportion of viable cells measured by crystal violet assay.

x. To Evaluate the Spread of OV-Chase ABC and 34.5N-Chase ABC on Glioma Spheres Grown as Ex Vivo Organotypic Cell Cultures.

The spread of an oHSV in glioma spheroids grown on organotypic brain slices can be evaluated in three different glioma cell lines. Glioma cell and primary tumor derived neurosphere cell spherical aggregates (G35, 157GBM and GBM30) can be individually “seeded” onto the brain slices with a capillary pipette and cultured for an additional 72 to 96 hours. The spheres can be infected with control viruses (HSVQ, or rQnestin34.5) or huChase ABC expressing viruses (OV-huChase ABC and 34.5N-huChase ABC). Spread of oHSV in the sphere can be visualized by confocal fluorescent imaging of oHSV encoded GFP in infected cells over a period of time. Infection was found to be apparent only in the rim of the spheres infected with rHSVQ1, but spheroids treated with OV-Chase ABC showed increased spread to the core of spheres over a period of time (24 hrs: top, 48 hrs: middle, 60 hrs: bottom row) (FIG. 16).

xi. Evaluate the Therapeutic Efficacy of Chase ABC Expressing oHSV Against Intracranial Glioma:

The therapeutic efficacy of control viruses (rHSVQ1 or rQnestin34.5) or huChase ABC expressing viruses (OV-huChase ABC and 34.5N-huChase ABC) can be tested using human glioma xenograft implanted intra-cerebrally in immune compromised mice (nu/nu). Intracranial tumors can be established by injecting 2×10⁵ of one of the GSs (FIG. 24) into nu/nu mice brains. Seven days after tumor implantation, 1×10⁵ of either control viruses (HSVQ, or rQnestin34.5) or huChase ABC expressing viruses (OVhuChase ABC and 34.5N-huChase ABC) can be injected by direct intratumoral injection. Glioma bearing mice can be treated with 10⁴-10⁵ pfu of virus/mouse. Mice can be evaluated for increased survival by Kaplan Meier analysis. The animals can be monitored closely for any signs of toxicity due to virus. This experiment can be repeated in a syngeneic mouse glioma model (4C8 cells in B6D2F1 mice) to evaluate the effect of treatment with OV-Chase ABC in an immuno-competent animal model. This model has been previously shown to be a relevant model for evaluating efficacy of oncolytic HSV43. This model has been tested in vitro for increased nestin expression and sensitivity to OV. The histology of the tumors produced by these cells in B6D2F1 mice was examined based on the presence of endothelial proliferation, necrosis and invasion it recapitulated histopathology of human GBMs.

xii. Impact of Chase Treatment on oHSV Spread In Vivo:

157GBM and 4C8 glioma can be implanted intracranially in athymic nude mice and in syngeneic B6D2F1 mice, respectively. Mice can be randomized to receive intra-tumoral control viruses (rHSVQ1, or rQNestin34.5) or huChase ABC expressing viruses (OVhuChase ABC and 34.5N-huChase ABC). Twenty-four and 48 hours after oHSV delivery, the mice can be sacrificed and virus bearing tumor sections can be compared for virus spread by immuno-histochemistry or immuno-fluorescence for GFP encoded by virus.

xiii. Ex Vivo Comparison of Spread of oHSV and Inert (Non CSPG Binding) Quantum Dots:

If release of oHSV bound to CSPG contributes to Chase ABC mediated enhancement can be tested by comparing the spread of inert quantum dot-encoded silica microspheres to oHSV. These inert quantum dot-silica microspheres do not bind to CSPG and thus, Chase ABC treatment should not affect their bioavailability. These dots have been used before to simulate the physical spread of HSV-1 virions in vivo. Briefly, GS can be cultured on inserts and treated with buffer or Chase ABC for 24 hrs prior to inoculation with oHSV (rQNestin34.5 or rHSVQ1) or quantum dot-encoded silica microspheres. The spread of OV and quantum dots through the tissue can be evaluated by confocal fluorescence microscopy. The depth of tissue penetration can be compared for oHSV and quantum dots, at 4, 6, 8, and 10 hours after treatment (prior to viral replication). If Chase ABC treatment is affecting bio-availability of oHSV and increasing its spread, Chase ABC treatment can improve the spread of oHSV compared to its effect on spread of labeled inert nano-particles.

xiv. In Vivo Comparison of Spread of oHSV and Inert (Non CSPG Binding) Quantum Dots:

Intracranial tumors can be used in nude mice (157GBM) and 4C8 glioma cells in syngeneic B6D2F1 mice to evaluate if Chase mediated enhancement of oHSV spread in vivo is due to disruption of a physical barrier, and/or due to release of CSPG bound virus. The spread of non CSPG binding inert quantum dot-encoded silica microspheres can be compared to oHSV in tumor tissue. Briefly, mice can be randomized to receive intra-tumoral buffer of Chase using Alzet mini pumps. 24 hrs after treatment mice can be injected with single dose of oHSV (rQNestin34.5 or rHSVQ1), and quantum dot-encoded silica microspheres injected at a constant flow rate of 1 ul/10 minutes. Animals can be sacrificed 6 hours later and tumor sections can be compared for Chase ABC mediated enhancement of virus and nano-particles. If apart from physical opening of the barrier, Chase ABC is also affecting bio-availability of virus then the increase in spread of oHSV after Chase ABC treatment can be better than that achieved by Chase ABC treatment of nano particles.

xv. Statistical Considerations:

To compare in vitro effect of different viruses Chase ABC, n=9 for each group (CV=30%, α=0.001) can be used to achieve 80% power in detecting 2 fold change. To compare in vivo effect of rQnestin34.5 with 34.5N-Chase ABC, n=11 (CV=50%, α=0.003) per group can be used to achieve 80% power to detect 2.4 fold change. To test the effect of Chase ABC on OV bioavailability, a 2 by 2 design can be used to show that the depth of tissue penetration can be increased more by Chase ABC when OV is used as compared to quantum dots. For this interaction contrast, in order to achieve 80% power in virus spread under Chase ABC vs. buffer in ex vivo C1.2.8, n=10 glioma in each group (CV=50%, α=0.013) can be used for a 3.5 fold detection; for in vivo (C.1.2.9), n=14 mice in each group (CV=50%, α=0.05) can be used for a 2.3 fold detection. ANOVA can be used to analyze data for experiments in this aim. Survival studies can be performed to compare the therapeutic efficacy of two virus types. Logrank test can be employed to test the survival curve difference. With n=10 mice per group, more than 80% power to detect a reduction from 80% to 20% in deaths at day 30 for 34.5N-Chase ABC over rQnestin34.5 in the Kaplan-Meier curves with α=0.006 can be achieved.

xvi. Alternatives

The panel of glioma cells can be increased and determined to see if this correlates with overall CSPG expression from these cells. Expression of a certain type of CSPG (Aggrecan, Brevican, Versican etc) can correlate with reduced viral spread. If gC/gB modifications in retargeted oHSV can further augment the spread and efficacy of Chase expressing oHSV can be evaluated.

2. The Magnitude and Type of Tumor Micro-Environmental Changes Caused by OVChase Expression and its Effects on Tumor and Normal Brain

Characterize the magnitude and type of tumor micro-environmental changes caused by Chase ABC expression and its effects on tumor and normal brain. Treatment of glioma CSPG with Chase ABC can change the tumor microenvironment by altering perfusion and vasculature. Understanding how Chase ABC expression alters glioma biology ensures its safety in the context of oHSVmediated delivery.

i. Characterization of the effect of Chase ABC expressed by oHSV on glioma cell dispersal.

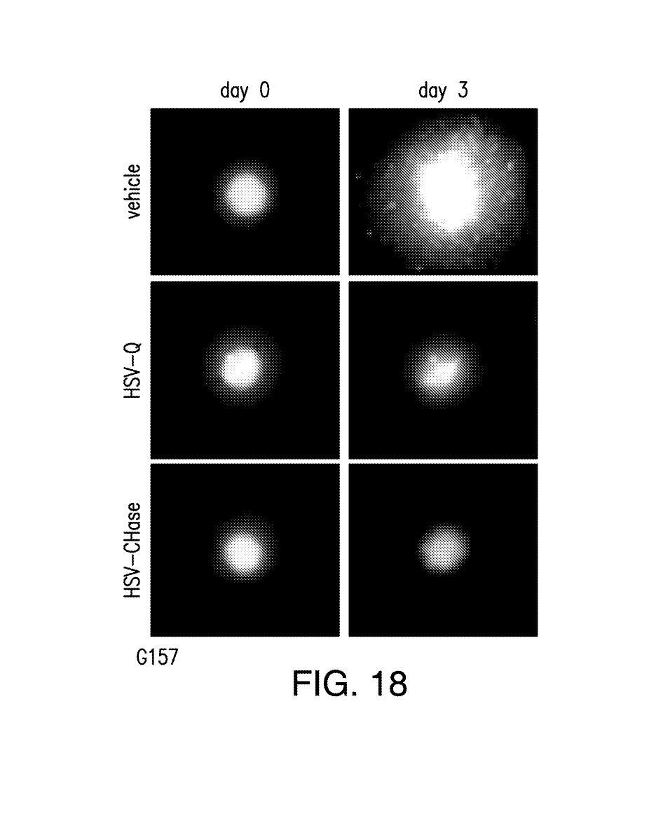

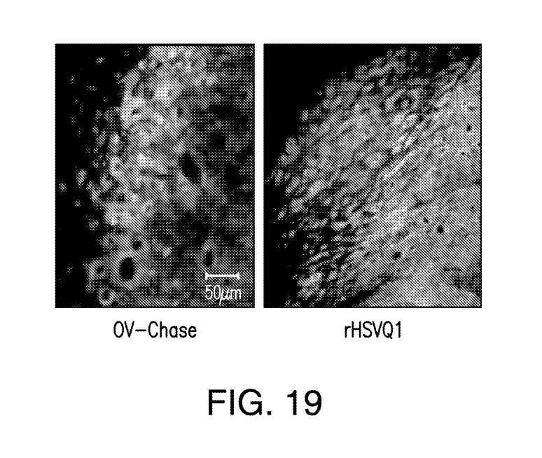

Since CSPGs have been shown to be involved in control of motility of neurons in developing and adult CNS, and have been shown to increase glioma cell invasion, whether degradation of GAG chains by Chase ABC affects invasiveness of glioma cells into the surrounding matrix in in vitro and in vivo models can be investigated. OV-Chase did not increase invasion of glioma cell lines in vitro and in vivo. Since standard glioma cell lines do not form invasive tumors in vivo, the ability of OV-Chase to affect migration of 157GBM and G35 primary GBM derived neuro-sphere cells was examined (FIG. 18). These cells are highly invasive and form invasive GBM like tumors in nude mice. RFP labeled 157GBM (FIG. 18) and G35 GS treated with PBS, rHSVQ1 or OVChase, were cultured onto organotypic brain slices. The spread of spheres over time into the brain slice was calculated as an index of invasion. Both neuro-spheres infected with rHSVQ1 or OV-Chase showed reduced invasion into the brain slice compared to untreated spheroids (p<0.05). Quantification of these showed there was no significant difference in the invasion of neurospheres treated with rHSVQ1 or OV-Chase.

ii. Effect of Chase ABC oHSV on Glioma Dispersal In Vivo:

Inoculation of intracranial glioma U87ΔEGFR with Chase ABCexpressing virus did not increase infiltration of malignant cells into brain parenchyma compared to PBS- or rHSVQ1 treated tumors. However these are in general non invasive tumors and so to validate if Chase expressing virus affected invasion in vivo mice bearing invasive intracranial GBM (G68; FIG. 24) were treated with rHSVQ1 or OV-Chase. Images of the invasive front of tumors (harvested 6 days after treatment) showed no difference in invasion between tumors treated with rHSVQ1, or OV-Chase.

iii. Removal of chondroitin sulfated GAG chains from CSPG would likely modulate the tumor microenvironment.

Since changes in the tumor microenvironment could affect tumor biology and outcome, the impact of this treatment on tumor vascular biology, tumor cell invasion, and safety can be evaluated. To validate the findings the effect of Chase ABC expressing virus on glioma biology using invasive GSs can be evaluated. Its safety in vivo can also be evaluated.

iv. Characterize the Effect of 34.5N-Chase ABC on Glioma Cell Dispersal.

Effect of 34.5N-Chase ABC on glioma cell haptotaxis and invasion can be tested using a modified Boyden-Chamber assay. Briefly, glioma cells (no serum) infected with control viruses (rHSVQ1, or rQNestin34.5) or huChase ABC expressing viruses (0V-huChase ABC and 34.5N-huChase ABC) or PBS treated can be added to the inner chamber of the multiwell inserts (8 μm, uncoated or coated with atrigel). The outer chambers can be filled with culture medium containing 10% FBS as chemo-attractant. After 6 hours, the cells remaining in the inner chamber can be removed with a cotton swab. Migrated cells on the other side of the inserts can be fixed and stained with crystal violet and imaged with an inverted microscope (10× magnification, six tiled pictures/well). Total number of nuclei in the images can be automatically counted using ImageJ software.

Effects on cell dispersal in organotypic brain slice cultures: Organotypic cultures can be performed (see FIG. 18). Fluorescently labeled GS can be individually “seeded” onto the brain slices with a capillary pipette and cultured for an additional 72 to 96 hours. The spheres can be infected with control viruses (rHSVQ1, or rQNestin34.5) or huChase ABC expressing viruses (OV-huChase ABC and 34.5N-huChase ABC). Confocal images of the fluorescent cells infiltrating into the brain slice can be obtained at 24 h intervals using an inverted confocal microscope Leica DM/IRE2. ImageJ software can be used to quantify the total volume occupied by the glioma cells over time. Experiments can be performed in triplicate, using 10 aggregates per experimental condition. Dispersion volumes for each experimental condition can be adjusted as percentage of controls and analyzed by 2-way ANOVA for repeated measures to determine differences between rQnestin34.5 and 34.5N-huChase ABC expressing cells at different times post-seeding.

Effects on glioma cell dispersion in vivo: Ten cresyl-violet stained sections from each tumor can be used to quantify cell dispersion by manually counting the total number of glioma cell clusters at a distance >1 mm from the tumor border. Each tumor can be then identified by a dispersion index, calculated as the average number of clusters/section divided by the total volume of the tumor. Dispersion index values can be compared between control viruses (rHSVQ1, or rQNestin34.5) or huChase ABC expressing viruses (OVhuChase ABC and 34.5N-huChase ABC)-treated tumors, by 1-way ANOVA and post-hoc Tukey-Kramer test. 157GBM or other GS which form invasive and highly vascularized tumor in vivo can be used.

v. Evaluate Changes in Micro Vessel Density (MVD) in Intracranial Tumors in Mice Treated with Chase ABC Expressing OV.

IHC can be used to evaluate changes in MVD in intracranial tumors treated with Chase ABC expressing viruses compared to control viruses. It has been shown that oHSV therapy has a direct anti-angiogenic effect on tumor after treatment. It has also been shown that after virus clearance, when residual/untreated tumor re-grows, oHSV treated tumors have increased MVD. The impact of Chase ABC expressing virus on both direct and late effect after oHSV treatment can be evaluated. Mice (B6D2F1 or athymic nude mice) with established intracranial tumors (157GBM), in nude mice or 4C8 in B6D2F1 mice can be treated with PBS, 34.5N-huChase ABC or rQnestin34.5. 157GBM forms invasive and highly vascularized tumors in vivo. Tumors can be harvested at 7 days and at time of death after oHSV treatment and sections can be stained for CD31 (endothelial cell marker). Mean micro vessel density (MVD) can be calculated for each therapy group.

vi. Evaluate changes in vascular perfusion using DCE MRI in mice treated with N34.5-Chase ABC compared to rQNestin34.5.

Changes in perfusion can be measured by DCE MRI of mice bearing intracranial 157GBM treated with PBS, 34.5N-huChase ABC or rQNestin34.5. MR imaging of female athymic nude mice can be performed on the 9.4 T system (Bruker BioSpin; Billerica, Mass.) using a mouse brain surface coil and 70 mm diameter linear rat coil. 3 sets of coronal images of the brain can be acquired prior to a bolus injection of 1 mmol/kg Gd-DPTA delivered within 30 sec through a tail vein catheter. The dynamic MRI data can be obtained using a FLASH pulse sequence TR/TE=150/2.4 ms, FA=50o, NEX=1. Temporal image data can be collected for 40 minutes following the Gd injection. The carotid arteries can be used to determine the arterial input function and the mean Gd time course can be determined using the method outlined in McIntyre et al. The model of Tofts and Kermode 57 can be used for the DCE-MRI data analysis and the calculations of Ktrans and ve. All analysis routines have been developed using C programming and in-house software. Changes in Ktrans in animals pre- and post oHSV treatment on days 3, 5, and 7 can be calculated to evaluate changes in vascular leakage with huChase ABC expressing OV.

vii. Evaluate safety of intra-cerebral injection of oHSV in non tumor bearing Balb/c mice.

If 34.5N-huChase ABC leads to animal death/toxicity after intracerebral administration can be determined Into the brains of Balb/c mice 10³, 10⁴, 10⁵, 10⁶, and 10⁷ pfus of 34.5N-huChase ABC can be inoculated. An LD50 for F strain at 10⁴ pfus can be seen, while no deaths result for 34.5N-huChase ABC up to 10⁶ pfus (based on safety data from rQNestin34.5). If toxicity occurs, an additional 5 animals can be treated in the cohort. Brains from animals can be saved for histological studies. Toxicity can be determined by death or occurrence of neurological signs (poor grooming behavior, hemiparesis, lethargy) or loss of body weight greater than 20%. Brains from necropsied animals as well as liver, spleen, lungs, heart, gonads, trigeminal ganglia can be sectioned and analyzed Immunohistochemical stains for CD68 (mononuclear cells/microglia), NKRP 1 (NK cells), CD4+ and CD8+ T cells, and CD45 (general pan lymphocyte marker) can also be carried out in the brain to visualize the acute phase response. These experiments can thus elucidate the possible toxicity under a variety of routes of administration.

viii. Statistical Considerations:

To compare glioma cell dispersal effect of 34.5N-Chase ABC with rQnestin34.5 in vivo, n=5 mice per group (CV=30%, α=0.05) can be used to achieve 80% power to detect 2.6 fold change. To compare in vivo effect of 34.5N-Chase ABC with rQnestin34.5 on microenvironment, in order to achieve 80% power to detect 2.2 fold difference in microvessel density, n=7 mice for each group (CV=50%, α=0.05) can be used; in vascular perfusion measured by Ktrans, n=10 mice for each group (CV=50%, α=0.017) can be used to detect a 2.2 fold change, and two-way ANOVA (two factors: treatment, time) with Holm's adjustment for multiple comparisons can be employed for data analysis. n=3-5 mice can be used for PBS treatment as control.

ix. Alternatives

The dose and time after virus delivery can be optimized to have viable tumor to evaluate changes in tumor biology after oHSV treatment. If Chase ABC treatment increases glioma angiogenesis, then the combination with pharmacologic agents targeting those aspects of glioma biology can be evaluated to determine if they will synergize to increase therapeutic efficacy in vivo. To rule out the possibility of a mutation occurring during construction of the recombinant plasmid several recombinant viruses can be isolated by plaque purification and the viruses compared for infection and replication potential. The isolated BAC can also be reverted back to rescue the virus and compared to the original HSVQ virus for its infection and replication.

3. The Impact of Chase ABC-Expressing Virotherapy on the Infiltration and Activation Status of Natural Killer (NK) and Macrophage Cells.

Determine the impact of Chase ABC-expressing virotherapy on tumoral infiltration and activation status of NK and macrophage cells. Treatment of glioma CSPG with chondroitinase can alter the infiltration of NK cells and other immune mediators within oHSV infected gliomas and possibly impacts the invasion/migration of tumor cells themselves. Chondoitin sulfate disaccharides released after enzymatic digestion of CSPGs with Chase ABC have been shown to have an anti-inflammatory effect in the periphery and in the CNS3. In the central nervous system, CS can reduce inflammatory response of astrocytes, by reducing production of TNF-alpha, iNOS and COX-259. This disaccharide degradation product of Chase has also been shown to be a bioactive regulator of microglia and macrophage function suggesting it may have therapeutic implications for CNS immune related disorders. Thus collectively, there is significant data indicating that CS released after Chase treatment has immune-modulatory effects. It has been shown that macrophages and microglia play a critical role in oHSV clearance in vivo, thus the impact of Chase expressing viruses on host immune responses after treatment in a syngeneic mouse model can be studied. Since macrophages and microglia also affect NK cells which can have an impact on oHSV therapy the impact of Chase on NK cell activation can also be investigated.

i. Changes in Host NK Cell Infiltration after oHSV Therapy:

The infiltration of NK cells in athymic nude mice implanted intracranially with 10⁵ U87ΔEGFR cells was examined. There was a significant increase in NK cell infiltration of intracranial tumors following oHSV treatment. Importantly, NK depletion (using the well-described anti-Asialo GM-1 mAb treatment) enhanced oHSV therapy. Overall survival was significantly enhanced after depletion of NK cells in human U87ΔEGFR or syngeneic 4C8 glioma bearing mice treated with rQNestin34.5, indicating the significance of this innate immune cell population in limiting oHSV efficacy.

ii. Effect of Choindroitinase on Virus Replication In Vivo:

The effect of Chase ABC expression on virus replication in vivo can be evaluated by comparing the number of infectious virus particles present in tumor, treated with control viruses (rHSVQ1, or rQNestin34.5) or huChase ABC expressing viruses OVhuChase ABC and 34.5N-huChase ABC). One immuno-competent tumor model (4C8 lioma cells grown in B6D2F1) can be treated with PBS, or the indicated virus (day 7 post tumor implantation). Tumor tissue can be harvested on days 1, 3, 5, and 7 after treatment and the number of infectious virus particles can be measured by a standard plaque formation assay to identify the number of infectious viral particles in each group. As a control measuring if Chase ABC expression alters replication of the viruses in vitro in these cells can be performed.

iii. Compare the infiltration of peripheral phagocytic cells in brain tumors treated with PBS, 34.5NChase ABC or rQnestin34.5.

The impact of Chase ABC treatment on tumor infiltration of macrophages in a syngeneic mouse glioma model using MRI imaging of phagocytosed monocrystalline iron oxide (MIO) particles can be examined Circulating phagocytic cells engulf these particles, while unengulfed particles clear out of the circulation in 24 hours. The phagocytic cells that have engulfed MIO particles can then be imaged by MRI. The intensity of the T2* signal can be quantified and compared between the groups.

iv. Compare the infiltration of monocytic, microglial and NK cells into intracranial tumors of mice treated with PBS, 34.5N-Chase ABC or rQNestin34.5.

Mice (B6D2F1) with established intracranial tumors (4C8) can be treated with PBS, 34.5N-Chase ABC or rQnestin34.5. Tumors can be harvested 24, 48 and 72 after treatment and the infiltration of NK cells, monocyte-macrophage cells and microglial cells can be evaluated by flow cytometry using DX5, (NK cells) Cd11b+/CD45high (monocytic macrophages) and Cd11b+CD45low (microglia). The infiltration of NK cells and monocytic and microglial cells in tumor bearing hemisphere of athymic nude mice implanted intracranially with 10⁵ U87ΔEGFR cells was examined.

v. Evaluate Changes in Monocyte/Microglial Cell Activation/Maturation Status.

Mice (B6D2F1) with established syngeneic intracranial tumors (4C8) can be treated with PBS, 34.5N-Chase ABC or rQnestin34.5. Tumors can be harvested 24, 48 and 72 after treatment and the infiltration of monocytic-macrophage cells and microglial cells can be evaluated by flow cytometry using Cd11b+/CD45high (monocytic macrophages) and Cd11b+CD45low (microglia). These cells can be further evaluated for changes in their expression levels of Ly6C, MHCII, and CD206 to evaluate changes in their activation.

vi. Changes in NK maturational status in response to treatment with 34.5N-Chase ABC.

Changes in NK cell maturation state in vivo can be analyzed. The use of CD11b and CD27 surface markers to classify NK cells into three distinct groups has been done. NK cell differentiation in mice has been found to develop from a relatively immature CD27highC11blow state, to the double positive CD27highCD11bhigh, and ultimately to the senescent CD27lowCD11bhigh. While investigators have begun to explore the normal anatomical distribution for each stage of NK cell, there has not been an analysis of the maturation state of NK cells and their respective cytotoxic capacities within the GBM microenvironment either in the absence or presence of oHSV. Thus, mice can be sacrificed at both 24 and 72 hr post-infection following intracranial tumor implantation with 4C8, followed by control viruses (rHSVQ1, or rQNestin34.5) or huChase ABC expressing viruses (OV-huChase ABC and 34.5N-huChase ABC) or PBS administration.

vii. Statistical Considerations:

To compare in vivo effect of 34.5N-Chase ABC with rQnestin34.5 on virus replication and phagocytosis and immune cell infiltration and NK maturation, n=10 mice per group (CV=50%, α=0.013) can be used for a 2.2 fold detection in replication and n=7 mice per group (CV=50%, α=0.05) can be used for a 2.3 fold detection in phagocytosis and n=10 mice per group (CV=50%, α=0.0167) for a 2.2 fold detection in immune cell infitration and NK maturation with 80% power. Two-way ANOVA (two factors: treatment, time) with Holm's adjustment for multiple comparisons can be employed for data analysis. n=3-5 mice can be used for PBS treatment which serves as the technique control. To study the impact of CSPG-DS on antiviral host response, n=11 mice per group (CV=50%, α=0.025) can be used to detect 2 fold change with 80% power.

viii. Alternatives

rQNestin34.5 can be compared to 34.5N-Chase ABC. However if results show either reduced efficacy or increased toxicity of 34.5-N-Chase ABC, the first generation OV-Chase ABC and HSVQ can be used in these studies. Chase ABC repeated injections in brain have not been associated with deleterious effects. In the event that Chase ABC produced by oHSV is found to be deleterious, oHSV replication and generation of Chase can be shut-off using antiviral herpetic drugs, such as valcyclovir.

It will be apparent to those skilled in the art that various modifications and variations can be made in the present invention without departing from the scope or spirit of the invention. Other aspects of the invention will be apparent to those skilled in the art from consideration of the specification and practice of the invention disclosed herein. It is intended that the specification and examples be considered as exemplary only, with a true scope and spirit of the invention being indicated by the following claims.

### REFERENCES

1. Moon L D, Asher R A, Rhodes K E, Fawcett J W: Regeneration of CNS axons back to their target following treatment of adult rat brain with chondroitinase ABC, Nat Neurosci 2001, 4:465-466
2. Rhodes K E, Fawcett J W: Chondroitin sulphate proteoglycans: preventing plasticity or protecting the CNS?, J Anat 2004, 204:33-48
3. Rolls A, Cahalon L, Bakalash S, Avidan H, Lider O, Schwartz M: A sulfated disaccharide derived from chondroitin sulfate proteoglycan protects against inflammation-associated neurodegeneration, Faseb J 2006, 20:547-549
4. Crespo D, Asher R A, Lin R, Rhodes K E, Fawcett J W: How does chondroitinase promote functional recovery in the damaged CNS?, Exp Neurol 2007,
5. Garcia-Alias G, Barkhuysen S, Buckle M, Fawcett J W: Chondroitinase ABC treatment opens a window of opportunity for task-specific rehabilitation, Nat Neurosci 2009, 12:1145-1151
6. Bruckner G, Bringmann A, Hartig W, Koppe G, Delpech B, Brauer K: Acute and long-lasting changes in extracellular-matrix chondroitin-sulphate proteoglycans induced by injection of chondroitinase ABC in the adult rat brain, Exp Brain Res 1998, 121:300-310
7. Dmitrieva N, Yu L, Viapiano M, Cripe T P, Chiocca E A, Glorioso J C, Kaur B: Choindroitinase ABC Imediated enhancement of oncolytic virus spread and anti tumor efficacy, Clin Cancer Res 2010,
8. Deal watch: Amgen buys oncolytic virus company, Nat Rev Drug Discov 2011, 10:166
9. Garber K: China approves world's first oncolytic virus therapy for cancer treatment, J Natl Cancer Inst 2006, 98:298-300
10. Bruckner G, Grosche J, Schmidt S, Hartig W, Margolis R U, Delpech B, Seidenbecher C I, Czaniera R, Schachner M: Postnatal development of perineuronal nets in wild-type mice and in a mutant deficient in tenascin-R, J Comp Neurol 2000, 428:616-629
11. Prabhakar S, Messerli S M, Stemmer-Rachamimov A O, Liu T C, Rabkin S, Martuza R, Breakefield X O: Treatment of implantable NF2 schwannoma tumor models with oncolytic herpes simplex virus G47Delta, Cancer Gene Ther 2007, 14:460-467
12. Hardcastle J, Kurozumi K, Chiocca E A, Kaur B: Oncolytic viruses driven by tumor-specific promoters, Curr Cancer Drug Targets 2007, 7:181-189
13. Kambara H, Okano H, Chiocca E A, Saeki Y: An oncolytic HSV-1 mutant expressing ICP34.5 under control of a nestin promoter increases survival of animals even when symptomatic from a brain tumor, Cancer Res 2005, 65:2832-2839
14. Kambara H, Saeki Y, Chiocca E A: Cyclophosphamide allows for in vivo dose reduction of a potent oncolytic virus, Cancer Res 2005, 65:11255-11258
15. Mahller Y Y, Williams J P, Baird W H, Mitton B, Grossheim J, Saeki Y, Cancelas J A, Ratner N, Cripe T P: Neuroblastoma cell lines contain pluripotent tumor initiating cells that are susceptible to a targeted oncolytic virus, PLoS ONE 2009, 4:e4235
16. Yoo J Y, Haseley A, Bratasz A, Chiocca E A, Zhang J, Powell K, Kaur B: Antitumor Efficacy of 34.5ENVE: A Transcriptionally Retargeted and “Vstat120”-expressing Oncolytic Virus, Mol Ther 2011,
17. Bellail A C, Hunter S B, Brat D J, Tan C, Van Meir E G: Microregional extracellular matrix heterogeneity in brain modulates glioma cell invasion, Int J Biochem Cell Biol 2004, 36:1046-1069
18. Delpech B, Maingonnat C, Girard N, Chauzy C, Maunoury R, Olivier A, Tayot J, Creissard P: Hyaluronan and hyaluronectin in the extracellular matrix of human brain tumour stroma, Eur J Cancer 1993, 29A:1012-1017
19. Vargova L, Homola A, Zamecnik J, Tichy M, Benes V, Sykova E: Diffusion parameters of the extracellular space in human gliomas, Glia 2003, 42:77-88
20. Moon L D, Asher R A, Fawcett J W: Limited growth of severed CNS axons after treatment of adult rat brain with hyaluronidase, J Neurosci Res 2003, 71:23-37
21. Sugimura T, Kato F, Mimatsu K, Takenaka O, Iwata H: Experimental chemonucleolysis with chondroitinase ABC in monkeys, Spine 1996, 21:161-165
22. Kuriyama N, Kuriyama H, Julin C M, Lamborn K, Israel M A: Pretreatment with protease is a useful experimental strategy for enhancing adenovirus-mediated cancer gene therapy, Hum Gene Ther 2000, 11:2219-2230
23. Hong Y, Ho K S, Eu K W, Cheah P Y: A susceptibility gene set for early onset colorectal cancer that integrates diverse signaling pathways: implication for tumorigenesis, Clin Cancer Res 2007, 13:1107-1114
24. McKee T D, Grandi P, Mok W, Alexandrakis G, Insin N, Zimmer J P, Bawendi M G, Boucher Y, Breakefield X O, Jain R K: Degradation of fibrillar collagen in a human melanoma xenograft improves the efficacy of an oncolytic herpes simplex virus vector, Cancer Res 2006, 66:2509-2513
25. Rosenberg G A, Kornfeld M, Estrada E, Kelley R O, Liotta L A, Stetler-Stevenson W G: TIMP-2 reduces proteolytic opening of blood-brain barrier by type IV collagenase, Brain Res 1992, 576:203-207
26. Ganesh S, Gonzalez-Edick M, Gibbons D, Van Roey M, Jooss K: Intratumoral coadministration of hyaluronidase enzyme and oncolytic adenoviruses enhances virus potency in metastatic tumor models, Clin Cancer Res 2008, 14:3933-3941
27. Struve J, Maher P C, Li Y Q, Kinney S, Fehlings M G, Kuntz Ct, Sherman L S: Disruption of the hyaluronan-based extracellular matrix in spinal cord promotes astrocyte proliferation, Glia 2005, 52:16-24
28. Viapiano M S, Lawler S E: Glioma invasion: Mechanisims and therapeutic challenges. Edited by Van meir EG. New jersey, Humana Press, 2008, p.
29. Yamagata T, Saito H, Habuchi O, Suzuki S: Purification and properties of bacterial chondroitinases and chondrosulfatases, J Biol Chem 1968, 243:1523-1535
30. Viapiano M S, Bi W L, Piepmeier J, Hockfield S, Matthews R T: Novel tumor-specific isoforms of BEHAB/brevican identified in human malignant gliomas, Cancer Res 2005, 65:6726-6733
31. Arslan F, Bosserhoff A K, Nickl-Jockschat T, Doerfelt A, Bogdahn U, Hau P: The role of versican isoforms V0/V1 in glioma migration mediated by transforming growth factor-beta2, Br J Cancer 2007, 96:1560-1568
32. Gladson C L: The extracellular matrix of gliomas: modulation of cell function, J Neuropathol Exp Neurol 1999, 58:1029-1040
33. Ulbricht U, Eckerich C, Fillbrandt R, Westphal M, Lamszus K: RNA interference targeting protein tyrosine phosphatase zeta/receptor-type protein tyrosine phosphatase beta suppresses glioblastoma growth in vitro and in vivo, J Neurochem 2006, 98:1497-1506
34. Rolls A, Avidan H, Cahalon L, Schori H, Bakalash S, Litvak V, Lev S, Lider O, Schwartz M: A disaccharide derived from chondroitin sulphate proteoglycan promotes central nervous system repair in rats and mice, Eur J Neurosci 2004, 20:1973-1983
35. Muir E M, Fyfe I, Gardiner S, Li L, Warren P, Fawcett J W, Keynes R J, Rogers J H: Modification of Nglycosylation sites allows secretion of bacterial chondroitinase ABC from mammalian cells, J Biotechnol 2010, 145:103-110
36. Fulci G, Dmitrieva N, Gianni D, Fontana E J, Pan X, Lu Y, Kaufman C S, Kaur B, Lawler S E, Lee R J, Marsh C B, Brat D J, van Rooijen N, Stemmer-Rachamimov A O, Hochberg F H, Weissleder R, Martuza R L, Chiocca E A: Depletion of peripheral macrophages and brain microglia increases brain tumor titers of oncolytic viruses, Cancer Res 2007, 67:9398-9406
37. Nowicki M O, Dmitrieva N, Stein A M, Cutter J L, Godlewski J, Saeki Y, Nita M, Berens M E, Sander L M, Newton H B, Chiocca E A, Lawler S: Lithium inhibits invasion of glioma cells; possible involvement of glycogen synthase kinase-3, Neuro Oncol 2008, 10:690-699
38. Adamiak B, Trybala E, Mardberg K, Johansson M, Liljeqvist J A, Olofsson S, Grabowska A, Bienkowska-Szewczyk K, Szewczyk B, Bergstrom T: Human antibodies to herpes simplex virus type 1 glycoprotein C are neutralizing and target the heparan sulfate-binding domain, Virology 2010, 400:197-206
39. Bergefall K, Trybala E, Johansson M, Uyama T, Naito S, Yamada S, Kitagawa H, Sugahara K, Bergstrom T: Chondroitin sulfate characterized by the E-disaccharide unit is a potent inhibitor of herpes simplex virus infectivity and provides the virus binding sites on gro2C cells, J Biol Chem 2005, 280:32193-32199
40. Mardberg K, Nystrom K, Tarp M A, Trybala E, Clausen H, Bergstrom T, Olofsson S: Basic amino acids as modulators of an O-linked glycosylation signal of the herpes simplex virus type 1 glycoprotein gC: functional roles in viral infectivity, Glycobiology 2004, 14:571-581
41. Tyminski E, Leroy S, Terada K, Finkelstein D M, Hyatt J L, Danks M K, Potter P M, Saeki Y, Chiocca E A: Brain tumor oncolysis with replication-conditional herpes simplex virus type 1 expressing the prodrugl activating genes, CYP2B1 and secreted human intestinal carboxylesterase, in combination with cyclophosphamide and irinotecan, Cancer Res 2005, 65:6850-6857
42. Kaur B, Cork S M, Sandberg E M, Devi N S, Zhang Z, Klenotic P A, Febbraio M, Shim H, Mao H, Tucker-Burden C, Silverstein R L, Brat D J, Olson J J, Van Meir E G: Vasculostatin inhibits intracranial glioma growth and negatively regulates in vivo angiogenesis through a CD36-dependent mechanism, Cancer Res 2009, 69:1212-1220
43. Hellums E K, Markert J M, Parker J N, He B, Perbal B, Roizman B, Whitley R J, Langford C P, Bharara S, Gillespie G Y: Increased efficacy of an interleukin-12-secreting herpes simplex virus in a syngeneic intracranial murine glioma model, Neurooncol 2005, 7:213-224
44. Theocharis A D, Skandalis S S, Tzanakakis G N, Karamanos N K: Proteoglycans in health and disease: novel roles for proteoglycans in malignancy and their pharmacological targeting, FEBS J 2010, 277:3904-3923
45. Hayatsu N, Ogasawara S, Kaneko M K, Kato Y, Narimatsu H: Expression of highly sulfated keratin sulfate synthesized in human glioblastoma cells, Biochem Biophys Res Commun 2008, 368:217-222
46. Matthews R T, Gary S C, Zerillo C, Pratta M, Solomon K, Amer E C, Hockfield S: Brain-enriched hyaluronan binding (BEHAB)/brevican cleavage in a glioma cell line is mediated by a disintegrin and metalloproteinase with thrombospondin motifs (ADAMTS) family member, J Biol Chem 2000, 275:22695-22703
47. Viapiano M S, Matthews R T: From barriers to bridges: chondroitin sulfate proteoglycans in neuropathology, Trends Mol Med 2006, 12:488-496
48. Candolfi M, Curtin J F, Nichols W S, Muhammad A G, King G D, Pluhar G E, McNiel E A, Ohlfest J R, Freese A B, Moore P F, Lerner J, Lowenstein P R, Castro M G: Intracranial glioblastoma models in preclinical neuro-oncology: neuropathological characterization and tumor progression, J Neurooncol 2007, 85:133-148
49. Hu B, Kong L L, Matthews R T, Viapiano M S: The proteoglycan brevican binds to fibronectin after proteolytic cleavage and promotes glioma cell motility, J Biol Chem 2008, 283:24848-24859
50. Benencia F, Courreges M C, Conejo-Garcia J R, Buckanovich R J, Zhang L, Carroll R H, Morgan M A, Coukos G: Oncolytic HSV exerts direct antiangiogenic activity in ovarian carcinoma, Hum Gene Ther 2005, 16:765-778
51. Cinatl J, Jr., Michaelis M, Driever P H, Cinatl J, Hrabeta J, Suhan T, Doerr H W, Vogel J U: Multimutated herpes simplex virus g207 is a potent inhibitor of angiogenesis, Neoplasia 2004, 6:725-735
52. Mahller Y Y, Vaikunth S S, Currier M A, Miller S J, Ripberger M C, Hsu Y H, Mehrian-Shai R, Collins M H, Crombleholme T M, Ratner N, Cripe T P: Oncolytic HSV and erlotinib inhibit tumor growth and angiogenesis in a novel malignant peripheral nerve sheath tumor xenograft model, Mol Ther 2007, 15:279-286
53. Hardcastle J, Kurozumi K, Dmitrieva N, Sayers M P, Ahmad S, Waterman P, Weissleder R, Chiocca E A, Kaur B: Enhanced antitumor efficacy of vasculostatin (Vstat120) expressing oncolytic HSV-1, Mol Ther 2010, 18:285-294
54. Kurozumi K, Hardcastle J, Thakur R, Shroll J, Nowicki M, Otsuki A, Chiocca E A, Kaur B: Oncolytic HSV-1 infection of tumors induces angiogenesis and upregulates CYR61, Mol Ther 2008, 16:1382-1391
55. Aghi M, Rabkin S D, Martuza R L: Angiogenic response caused by oncolytic herpes simplex virusinduced reduced thrombospondin expression can be prevented by specific viral mutations or by administering a thrombospondin-derived peptide, Cancer Res 2007, 67:440-444
56. McIntyre D J, Ludwig C, Pasan A, Griffiths J R: A method for interleaved acquisition of a vascular input function for dynamic contrast-enhanced MRI in experimental rat tumours, NMR Biomed 2004, 17:132-143
57. Tofts P S, Kermode A G: Measurement of the blood-brain barrier permeability and leakage space using dynamic MR imaging. 1. Fundamental concepts, Magn Reson Med 1991, 17:357-367
58. Campo G M, Avenoso A, Campo S, D'Ascola A, Traina P, Sama D, Calatroni A: Purified human plasma glycosaminoglycans reduced NF-kappaB activation, pro-inflammatory cytokine production and apoptosis in LPS-treated chondrocytes, Innate Immun 2008, 14:233-246
59. Canas N, Gorina R, Planas A M, Verges J, Montell E, Garcia A G, Lopez M G: Chondroitin sulfate inhibits lipopolysaccharide-induced inflammation in rat astrocytes by preventing nuclear factor kappa B activation, Neuroscience 2010, 167:872-879
60. Ebert S, Schoeberl T, Walczak Y, Stoecker K, Stempfl T, Moehle C, Weber B H, Langmann T: Chondroitin sulfate disaccharide stimulates microglia to adopt a novel regulatory phenotype, J Leukoc Biol 2008, 84:736-740
61. Jin M, Iwamoto T, Yamada K, Satsu H, Totsuka M, Shimizu M: Disaccharide derived from chondroitin sulfate A suppressed CpG-induced IL-6 secretion in macrophage-like J774.1 cells, Cytokine 2010, 51:53-59
62. Movahedi K, Laoui D, Gysemans C, Baeten M, Stange G, Van den Bossche J, Mack M, Pipeleers D, In't Veld P, De Baetselier P, Van Ginderachter J A: Different tumor microenvironments contain functionally distinct subsets of macrophages derived from Ly6C(high) monocytes, Cancer Res 2010, 70:5728-5739
63. Kurozumi K, Hardcastle J, Thakur R, Yang M, Christoforidis G, Fulci G, Hochberg F H, Weissleder R, Carson W, Chiocca E A, Kaur B: Effect of tumor microenvironment modulation on the efficacy of oncolytic virus therapy, J Natl Cancer Inst 2007, 99:1768-1781
64. Fulci G, Breymann L, Gianni D, Kurozomi K, Rhee S S, Yu J, Kaur B, Louis D N, Weissleder R, Caligiuri M A, Chiocca E A: Cyclophosphamide enhances glioma virotherapy by inhibiting innate immne responses, Proc Natl Acad Sci USA 2006, 103:12873-12878
65. Hayakawa Y, Smyth M J: CD27 dissects mature NK cells into two subsets with distinct responsiveness and migratory capacity, J Immunol 2006, 176:1517-1524
66. Yu J, Wei M, Mao H, Zhang J, Hughes T, Mitsui T, Park I K, Hwang C, Liu S, Marcucci G, Trotta R, Benson D M, Jr., Caligiuri M A: CD94 defines phenotypically and functionally distinct mouse NK cell subsets, J Immunol 2009, 183:4968-4974

1. An isolated nucleic acid comprising the sequence of SEQ ID NO:1 or SEQ ID NO:2.

2. (canceled)

3. (canceled)

4. (canceled)

5. A recombinant protein comprising the amino acid sequence of SEQ ID NO:5 or SEQ ID NO:6.

6. (canceled)

7. (canceled)

8. (canceled)

9. (canceled)

10. (canceled)

11. (canceled)

12. (canceled)

13. (canceled)

14. A method of treating cancer comprising administering to a subject an effective amount of a composition comprising a mutant Chondroitinase ABC I protein sequence, or a nucleic acid sequence encoding the protein sequence, wherein the mutant Chondroitinase ABC I protein comprises SEQ ID NO:5 or SEQ ID NO:6.

15. (canceled)

16. (canceled)

17. The method of claim 14, wherein the composition comprises the nucleic acid sequence and a vector.

18. (canceled)

19. The method of claim 17, wherein the vector is an oncolytic viral vector.

20. The method of claim 17 wherein the oncolytic viral vector is herpes simplex virus type-1 (HSV-1).

21. The method of claim 14, wherein the nucleic acid sequence comprises SEQ ID NO:2.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. The method of claim 14 further comprising administering a cancer therapeutic.

34. The method of claim 33, wherein the cancer therapeutic is a chemotherapeutic agent.

35. A method of increasing the spread of HSV-1 comprising administering to a cell a composition comprising a mutant Chondroitinase ABC I protein sequence, or a nucleic acid sequence encoding the protein sequence, wherein the mutant Chondroitinase ABC I protein comprises SEQ ID NO:5 or SEQ ID NO:6.

36. The method of claim 35, wherein the composition comprises the nucleic acid sequence and a vector.

37. (canceled)

38. The method of claim 36, wherein the vector is a viral vector.

39. The method of claim 38, wherein the viral vector is an oncolytic viral vector.

40. The method of claim 35, wherein the nucleic acid sequence comprises SEQ ID NO:2.

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A method of treating a central nervous system injury comprising administering to a subject an effective amount of a composition comprising a mutant Chondroitinase ABC I protein sequence, or a nucleic acid sequence encoding the protein sequence, wherein the mutant Chondroitinase ABC I protein comprises SEQ ID NO:5 or SEQ ID NO:6.

54. (canceled)

55. (canceled)

56. The method of claim 53, wherein the composition comprises the nucleic acid and a vector.

57. (canceled)

58. The method of claim 53, wherein the nucleic acid sequence comprises SEQ ID NO:2.

59. (canceled)

60. (canceled)

61. (canceled)

62. (canceled)

63. (canceled)

64. (canceled)

65. (canceled)

66. (canceled)

67. (canceled)

68. (canceled)

69. The method of claim 53, wherein the mutant Chondroitinase ABC I protein increases axon regeneration.

70. The method of claim 53, wherein the central nervous system injury is a spinal cord injury.

71. (canceled)

72. (canceled)

73. (canceled)

74. (canceled)
